(19) United States
(12) Patent Application Publication
(10) Pub. No.: US 2025/0381106 A1
(43) Pub. Date: Dec. 18, 2025

(54) COMPOSITION FOR LIGHTENING KERATIN FIBRES AND PROCESS FOR LIGHTENING KERATIN FIBRES USING SAID COMPOSITION

(71) Applicant: L'OREAL, Paris (FR)

(72) Inventors: Nawel BAGHDADLI, AULNAY-SOUS-BOIS (FR); Stéphane SABELLE, AULNAY-SOUS-BOIS (FR); Stéphane BLAIS, AULNAY-SOUS-BOIS (FR); Rahma BENNI, AULNAY-SOUS-BOIS (FR)

(21) Appl. No.: 18/876,572

(22) PCT Filed: Jun. 21, 2023
(86) PCT No.: PCT/EP2023/066779

§ 371 Date: Dec. 18, 2024

(30) Foreign Application Priority Data
Jun. 22, 2022 (FR) 2206171

(51) Int. Cl. A61K 8/22; A61Q 5/06; A61Q 5/08; A61Q 5/10

(52) U.S. Cl. CPC A61K 8/22; A61Q 5/065; A61Q 5/08; A61Q 5/10; A61K 2800/43; A61K 2800/434; A61K 2800/882

(57) ABSTRACT

The present invention relates to a composition for lightening keratin fibres, comprising at least one chemical oxidizing agent, at least one (bi)carbonate, at least one silicate, at least one alcohol and at least one compound chosen from organic acids, amphoteric or zwitterionic compounds, esters and/or amides and/or imines, organic amines, alkanes and/or alkenes and/or ethers and mixtures thereof, and also to a process for lightening keratin fibres using said composition.

### TECHNICAL FIELD OF THE INVENTION

The present invention relates to a composition for lightening keratin fibres, comprising at least one chemical oxidizing agent, at least one (bi)carbonate, at least one silicate, at least one alcohol and at least one compound chosen from organic acids, amphoteric or zwitterionic compounds, esters and/or amides and/or imines, organic amines, alkanes and/or alkenes and/or ethers and mixtures thereof, and also to a process for lightening keratin fibres using said composition.

### BACKGROUND OF THE INVENTION

When a person wishes to change hair colour, notably when he or she wishes to obtain a lighter colour than their original colour, it is often necessary to perform preliminary lightening or bleaching of the hair. To do this, lightening or bleaching products are used. The lightening of head hair is evaluated by the “tone depth”, which characterizes the degree or level of lightening. The notion of “tone” is based on the classification of natural shades, one tone separating each shade from the shade immediately following or preceding it. This definition and the classification of natural shades are well known to hairstyling professionals and are published in the book Sciences des traitements capillaires [The Science of Hair Care] by Charles Zviak, 1988, published by Masson, pages 215 and 278.

The tone levels range from 1 (black) to 10 (very light blond), one unit corresponding to one tone; the higher the figure, the lighter the shade.

It is known practice to lighten or bleach the hair with lightening or bleaching compositions containing at least one chemical oxidizing agent, under alkaline pH conditions in the vast majority of cases. The role of this oxidizing agent is to degrade the melanin of the hair, which, depending on the nature of the oxidizing agent present and of the pH conditions, leads to more or less pronounced lightening of the fibres. Thus, for relatively mild lightening, the oxidizing agent is generally hydrogen peroxide. When greater lightening is desired, notably when the hair treated is dark, persulfates are usually used in the presence of hydrogen peroxide. However, the lightening obtained by the action of such a combination is not always satisfactory, since it produces hair with unattractive yellow-orange shades that are far removed from the natural shades, which complicates subsequent dyeing by limiting it to the production of warm tones. Furthermore, persulfate-based lightening compositions may lead to a deterioration of the fibre quality. Furthermore, persulfate-based compositions are generally not chemically compatible with the presence of oxidation dyes and/or direct dyes in order to be able to bleach the hair fibre and to dye it in a single step. Thus, when it is desired to simultaneously bleach and dye keratin fibres, a two-step process is used with a first step of bleaching the keratin fibres followed by a second step of dyeing the keratin fibres using a dye composition comprising one or more direct dyes and/or one or more oxidation dyes.

Thus, there is a real need to develop a composition which allows effective lightening of keratin fibres, notably dark keratin fibres, with a less yellow and more natural result. Such a composition should also be more respectful of the quality of the fibres, notably by minimizing their degradation. Finally, such a composition should also be compatible with the presence of direct dyes and/or oxidation dyes so as to obtain good colour buildup, intense and chromatic colours and also allow a wider range of colours to be obtained.

The Applicant has discovered, surprisingly, that all or some of these objectives can be achieved by means of the composition according to the present invention.

### SUMMARY OF THE INVENTION

According to a first aspect, a subject of the present invention is a composition comprising:
i) one or more chemical oxidizing agents chosen from hydrogen peroxide, hydrogen peroxide-generating systems other than peroxygenated salts, and mixtures thereof;
ii) one or more compounds chosen from carbonates, carbonate-generating systems, bicarbonates, bicarbonate-generating systems and mixtures thereof, preferably from carbonates, bicarbonates and mixtures thereof;
iii) one or more silicates;
iv) one or more alcohols;
iv′) one or more compounds different from the compounds iv), chosen from:
a) organic acids;
b) amphoteric or zwitterionic compounds chosen from: phospholipids such as lecithins, amino acids, amphoteric or zwitterionic surfactants and mixtures thereof;
c) esters, amides, imines and mixtures thereof;
d) organic amines;
e) compounds consisting of a saturated or unsaturated, linear or branched hydrocarbon-based chain comprising from 1 to 30 carbon atoms, the hydrocarbon-based chain being optionally substituted with one or more cyclic groups and/or optionally interrupted with one or more non-adjacent oxygen atoms; saturated or unsaturated, optionally aromatic, monocyclic or polycyclic, condensed or non-condensed carbocyclic compounds comprising from 3 to 22 carbon atoms, optionally substituted with one or more identical or different groups chosen from: (C₁-C₆)alkyl or (C₁-C₆)alkenyl or (C₁-C₆)alkoxy and/or optionally interrupted with one or more carbonyl groups (CO); monocyclic or polycyclic, condensed or non-condensed, saturated or unsaturated, notably aromatic heterocyclic compounds, comprising from 5 to 22 members and containing from 1 to 3 oxygen atoms, optionally substituted with one or more identical or different groups chosen from: (C₁-C₆)alkyl or (C₁-C₆)alkoxy and/or optionally interrupted with one or more carbonyl groups (CO) and mixtures thereof;
f) and mixtures thereof;

in which:
the silicate(s) are present in the composition in a total content ranging from 2% to 35% by weight relative to the total weight of the composition; and
the composition comprises a total content of persulfates of less than 5% by weight.

According to a second aspect, a subject of the present invention is a process for lightening keratin fibres, comprising the application to the keratin fibres of a composition comprising ingredients i) to iv) and iv′) as defined previously.

According to a third aspect, a subject of the present invention is a process for the simultaneous bleaching and dyeing of keratin fibres, comprising the application to the keratin fibres of a composition comprising ingredients i) to iv) and iv′) as defined previously and v) one or more colouring agents, preferably chosen from direct dyes, oxidation dyes and mixtures thereof.

According to a fourth aspect, a subject of the present invention is the use of a composition comprising ingredients i) to iv) and iv′) as defined previously for lightening keratin fibres, preferably for lightening keratin fibres while at the same time de-yellowing them.

According to a fifth aspect, a subject of the present invention is the use of a composition comprising ingredients i) to iv) and iv′) as defined previously and v) one or more colouring agents, preferably chosen from direct dyes, oxidation dyes and mixtures thereof, for the simultaneous bleaching and dyeing of keratin fibres.

According to a sixth aspect, a subject of the present invention is a device containing several separate compartments (kit), comprising:
a first compartment containing a composition (A) comprising:
one or more chemical oxidizing agents chosen from hydrogen peroxide, hydrogen peroxide-generating systems other than peroxygenated salts, and mixtures thereof, preferably hydrogen peroxide; and
optionally one or more alcohols iv) and/or one or more compounds iv′) as defined previously; and
a second compartment containing a composition (B) comprising:
one or more compounds chosen from carbonates, carbonate-generating systems, bicarbonates, bicarbonate-generating systems and mixtures thereof, preferably chosen from carbonates and bicarbonates, and mixtures thereof; and
one or more silicates; and
optionally one or more colouring agents, preferably chosen from direct dyes and oxidation dyes, and mixtures thereof; and
optionally one or more alcohols iv) and/or one or more compounds iv′) as defined previously; and
optionally a third compartment containing a composition (C) comprising:
one or more colouring agents, preferably chosen from direct dyes and oxidation dyes, and mixtures thereof; and
optionally one or more alcohols iv) and/or one or more compounds iv′) as defined previously;

it being understood that:
at least one of the compositions (A) or (B) or at least one of the compositions (A) or (B) or (C), if composition (C) is present, comprises one or more alcohols iv); and
at least one of the compositions (A) or (B) or at least one of the compositions (A) or (B) or (C), if composition (C) is present, comprises one or more compounds iv′) as defined previously.

### BRIEF DESCRIPTION OF THE FIGURES

FIG. 1 is a graph representing the variation in intensity L* as a function of the parameter b* for the compositions according to the invention (Example 2) and the comparative persulfate-based compositions (Example 1), the values of L* and b* being measured in the CIE L*a*b* system.

### DETAILED DESCRIPTION OF THE INVENTION

For the purposes of the present invention and unless otherwise indicated:
the term “keratin fibres” means fibres of human or animal origin, such as head hair, bodily hairs, the eyelashes, the eyebrows, wool, angora, cashmere or fur. According to the present invention, the keratin fibres are preferably human keratin fibres, more preferentially head hair;
the term “alcohol” means an organic compound in which one of the carbon atoms is linked to an —OH hydroxyl or phenol group.
the term “ester” means a compound comprising at least one ester function.
the term “amide” means a compound comprising at least one amide function.
the term “imine” means a compound comprising at least one imine function.
the term “organic acid” means an organic compound comprising at least one —X(O)OH function in which X represents a carbon atom or S═O or P—OH, preferably a carbon atom.
the term “organic amine” means an organic compound comprising at least one primary, secondary or tertiary amine function.
the term “salt” means an addition salt with an organic or mineral acid or base. The addition salts with an acid are notably chosen from addition salts with acids such as hydrochloric acid, hydrobromic acid, sulfuric acid, citric acid, succinic acid, tartaric acid, lactic acid, tosylic acid, benzenesulfonic acid, phosphoric acid or acetic acid. The addition salts with a base are notably chosen from the addition salts with bases such as the basifying agents defined hereinbelow, notably alkali metal hydroxides, alkaline-earth metal hydroxides, ammonia, amines or alkanolamines.
the term “alkyl group” means a linear or branched saturated hydrocarbon-based radical comprising from 1 to 40 carbon atoms, preferably from 1 to 30 carbon atoms, more preferentially from 1 to 26 carbon atoms, even more preferentially from 1 to 22 carbon atoms, for example methyl, ethyl, n-propyl, isopropyl, butyl, n-pentyl, n-hexyl, n-decyl, n-dodecyl, tetradecyl, hexadecyl or eicosyl.
the term “(Cₓ-C_{y})alkyl group” means an alkyl group comprising from x to y carbon atoms.
the term “alkenyl group” means a linear or branched hydrocarbon-based radical comprising one or more ethylenic unsaturations, and from 1 to 30 carbon atoms, preferably from 1 to 26 carbon atoms, more preferentially from 1 to 22 carbon atoms, for example ethenyl, n-propenyl, isopropenyl, butenyl, n-pentenyl, n-hexenyl, n-decenyl, n-dodecenyl, tetradecenyl, hexadecenyl or eicosenyl.
the term “(Cₓ-C_{y})alkenyl group” means an alkenyl group comprising from x to y carbon atoms.
the term “alkoxy group” means an alkyl group bonded to an oxygen atom.
the term “(Cₓ-C_{y})alkoxy group” means an alkoxy group comprising from x to y carbon atoms.
the term “(poly)(hydroxy)alkyl group” means an alkyl group or a hydroxyalkyl group or a polyhydroxyalkyl group.
the term “hydroxyalkyl group” means an alkyl group substituted with a hydroxyl group (—OH);
the term “polyhydroxyalkyl group” means an alkyl group substituted with at least two hydroxyl groups (—OH).
the term “(poly)hydroxyalkyl group” means a hydroxyalkyl or polyhydroxyalkyl group.
the term “alkylamino group” means a group R′—NR^{a}— with R′ representing an alkyl group and R^{a} representing a hydrogen atom or a (C₁-C₄)alkyl group optionally substituted with one or more groups —X(O)OH with X representing a carbon atom or S═O or P—OH, preferably with X representing a carbon atom; R′ and R^{a} may form, with the nitrogen atom bearing them, a saturated or unsaturated cyclic group with 4 to 8 chain members, preferably 5 or 6 chain members, said cyclic group being optionally substituted, notably with one or more identical or different groups chosen from (C₁-C₄)(poly)hydroxyalkyl or (C₁-C₄)alkoxy groups.
the term “alkylamido group” means a group R″—NR^{a}—CO— with R″ representing an alkyl group and R^{a} representing a hydrogen atom or a (C₁-C₄)alkyl group optionally substituted with one or more groups —X(O)OH with X representing a carbon atom or S═O or P—OH, preferably with X representing a carbon atom; R″ and R^{a} may form, with the nitrogen atom bearing them, a saturated or unsaturated cyclic group with 4 to 8 chain members, preferably 5 or 6 chain members, said cyclic group being optionally substituted with, notably, one or more identical or different groups chosen from (C₁-C₄)(poly)hydroxyalkyl or (C₁-C₄)alkoxy groups.
the term “(hetero)cyclic group” means a cyclic or heterocyclic group.
the term “cyclic group” means a condensed or non-condensed, saturated or unsaturated, notably aromatic, monocyclic or polycyclic carbocycle comprising from 6 to 22 carbon atoms, it being possible for said cyclic group to be substituted with one or more identical or different groups, notably chosen from: (C₁-C₆)alkyl, (C₁-C₆)alkoxy, (C₁-C₆)(poly)hydroxyalkyl, hydroxyl (—OH) or carboxyl (—COOH).
the term “heterocyclic group” means a condensed or non-condensed, saturated or unsaturated, in particular aromatic, monocyclic or polycyclic group comprising from 5 to 22 members and containing from 1 to 3 heteroatoms chosen from a nitrogen, oxygen or sulfur atom, it being possible for said heterocyclic group to be substituted with one or more identical or different groups, notably chosen from: (C₁-C₆)alkyl, (C₁-C₆)alkoxy, (C₁-C₆)(poly)hydroxyalkyl, hydroxyl (—OH) or carboxyl (—COOH).
the term “silicate” means a silicic acid salt.
the term “colouring agent” means an oxidation dye, a direct dye or a pigment.
the term “oxidation dye” means an oxidation dye precursor chosen from oxidation bases and couplers. Oxidation bases and couplers are colourless or sparingly coloured compounds, which, via a condensation reaction in the presence of an oxidizing agent, give a coloured species;
the term “direct dye” means a natural and/or synthetic dye, including in the form of an extract or extracts, other than oxidation dyes. These are coloured compounds that will spread superficially on the fibre. They may be ionic or nonionic, i.e. anionic, cationic, neutral or nonionic.
the term “chemical oxidizing agent” means an oxidizing agent other than atmospheric oxygen.

Unless otherwise indicated, when compounds are mentioned in the present patent application, this also includes the optical isomers thereof, the geometrical isomers thereof, the tautomers thereof, the salts thereof or the solvates thereof such as hydrates, and mixtures thereof.

The terms “at least one” and “one or more” are synonymous and may be used interchangeably.

The terms “lightening” and “bleaching” are synonymous and may be used interchangeably.

### Composition

According to a first aspect, a subject of the present invention is a composition comprising ingredients i) to iv) and iv′) as defined previously.

The Applicant found, surprisingly, that the composition according to the present invention allowed effective lightening of keratin fibres with a less yellow and more natural result. When the colour of keratin fibres treated with the composition according to the invention is compared with the colour of keratin fibres treated with lightening compositions known in the art, b* values measured in the CIE L* a* b* system are observed to be lower for the composition according to the invention than for lightening compositions known in the art at an equivalent intensity level L*.

Moreover, the composition according to the invention is more respectful of the quality of the fibres, notably minimizing their degradation.

According to one preferred embodiment, the composition according to the invention comprises:
i) hydrogen peroxide;
ii) one or more compounds chosen from carbonates, bicarbonates and mixtures thereof;
iii) one or more silicates;
iv) one or more alcohols;
iv′) one or more compounds different from the compounds iv), chosen from:
a) organic acids;
b) amphoteric or zwitterionic compounds chosen from: phospholipids such as lecithins, amino acids, amphoteric or zwitterionic surfactants and mixtures thereof;
c) esters, amides, imines and mixtures thereof;
d) organic amines;
e) compounds consisting of a saturated or unsaturated, linear or branched hydrocarbon-based chain comprising from 1 to 30 carbon atoms, the hydrocarbon-based chain being optionally substituted with one or more cyclic groups and/or optionally interrupted with one or more non-adjacent oxygen atoms; saturated or unsaturated, optionally aromatic, monocyclic or polycyclic, condensed or non-condensed carbocyclic compounds comprising from 3 to 22 carbon atoms, optionally substituted with one or more identical or different groups chosen from: (C₁-C₆)alkyl or (C₁-C₆)alkenyl or (C₁-C₆)alkoxy and/or optionally interrupted with one or more carbonyl groups (CO); monocyclic or polycyclic, condensed or non-condensed, saturated or unsaturated, notably aromatic heterocyclic compounds, comprising from 5 to 22 members and containing from 1 to 3 oxygen atoms, optionally substituted with one or more identical or different groups chosen from: (C₁-C₆)alkyl or (C₁-C₆)alkoxy and/or optionally interrupted with one or more carbonyl groups (CO) and mixtures thereof;
f) and mixtures thereof;

it being understood that:
the silicate(s) are present in the composition in a total content ranging from 2% to 35% by weight relative to the total weight of the composition; and
the composition comprises a total content of persulfates of less than 5% by weight.

### Chemical Oxidizing Agents

The composition according to the invention comprises i) one or more chemical oxidizing agents chosen from hydrogen peroxide, hydrogen peroxide-generating systems other than peroxygenated salts and mixtures thereof.

The hydrogen peroxide-generating systems other than peroxygenated salts may be chosen from urea peroxide, polymeric complexes that can release hydrogen peroxide, oxidases, and mixtures thereof.

As examples of polymeric complexes that can release hydrogen peroxide, mention may be made of polyvinylpyrrolidone/H₂O₂ in particular in powder form, and the other polymeric complexes described in U.S. Pat. Nos. 5,008,093, 3,376,110 and 5,183,901.

Oxidases can produce hydrogen peroxide in the presence of a suitable substrate, for instance glucose in the case of glucose oxidase or uric acid with uricase.

According to a particular embodiment, hydrogen peroxide and/or the hydrogen peroxide-generating system(s) other than peroxygenated salts may be incorporated into the composition according to the invention just before it is applied to the keratin fibres. The intermediate composition(s) comprising hydrogen peroxide and/or hydrogen peroxide-generating system(s) other than peroxygenated salts may be referred to as oxidizing compositions and may also include various additional compounds or various adjuvants conventionally used in compositions for lightening keratin fibres.

According to a preferred embodiment, the composition according to the invention comprises hydrogen peroxide as chemical oxidizing agent.

The chemical oxidizing agent(s) are preferably present in the composition in a total content ranging from 1% to 12% by weight, more preferentially ranging from 3% to 9% by weight and even more preferentially ranging from 3.5% to 8.5% by weight, relative to the total weight of the composition.

According to a preferred embodiment, the hydrogen peroxide is present in the composition in a total content ranging from 1% to 12% by weight, preferably ranging from 3% to 9% by weight, more preferentially ranging from 3.5% to 8.5% by weight, relative to the total weight of the composition.

(Bi)Carbonates and/or (Bi)Carbonate-Generating Systems

The composition according to the invention also comprises ii) one or more compounds chosen from carbonates, carbonate-generating systems, bicarbonates, bicarbonate-generating systems and mixtures thereof.

According to a preferred embodiment, the composition according to the invention also comprises ii) one or more compounds chosen from carbonates, bicarbonates and mixtures thereof.

According to a more preferred embodiment, the composition according to the invention also comprises ii) one or more compounds chosen from ammonium carbonate, ammonium bicarbonate and mixtures thereof.

The compound(s) ii) are preferably present in the composition in a total content ranging from 0.01% to 20% by weight, preferably ranging from 1% to 15% by weight, more preferentially ranging from 2% to 15% by weight, even more preferentially ranging from 4% to 15% by weight, relative to the total weight of the composition.

Carbonates and/or Carbonate-Generating Systems

The term “carbonate-generating system” means a system which generates carbonate in situ, for instance carbon dioxide in water or percarbonate in water.

Preferably, the carbonate(s) are chosen from:
alkali metal carbonates;
alkaline-earth metal carbonates;
lanthanide carbonates;
transition metal carbonates;
bismuth carbonate;
cadmium carbonate;
thallium carbonate;
zinc carbonate;
the compounds of formula (N⁺R¹R²R³R⁴)₂CO₃²⁻ in which R¹, R², R³ and R⁴ represent, independently of each other, a hydrogen atom or a (C₁-C₄)alkyl group optionally substituted with a hydroxyl group;
guanidine carbonate;
mixtures thereof.

More preferentially, the carbonate(s) are chosen from sodium carbonate, potassium carbonate, caesium carbonate, lithium carbonate, magnesium carbonate, calcium carbonate, barium carbonate, strontium carbonate, cerium carbonate, lanthanum carbonate, yttrium carbonate, copper(II) carbonate, manganese carbonate, nickel carbonate, silver carbonate, zirconium carbonate, bismuth carbonate, cadmium carbonate, thallium carbonate, zinc carbonate, ammonium carbonate, guanidine carbonate, tetraethylammonium carbonate and mixtures thereof.

Even more preferentially, the carbonate(s) are chosen from sodium carbonate, potassium carbonate, caesium carbonate, magnesium carbonate, calcium carbonate, cerium carbonate, manganese carbonate, zinc carbonate, ammonium carbonate, guanidine carbonate and mixtures thereof.

Most preferentially, the carbonate(s) are chosen from sodium carbonate, potassium carbonate, magnesium carbonate, calcium carbonate, ammonium carbonate and mixtures thereof.

According to a particularly preferred embodiment, the carbonate included in the composition is ammonium carbonate.

The carbonate(s) and/or the carbonate-generating system(s) are preferably present in the composition in a total content ranging from 0.01% to 20% by weight, more preferentially ranging from 1% to 20% by weight and even more preferentially ranging from 1% to 10% by weight, relative to the total weight of the composition.

According to a preferred embodiment, the carbonate(s) are present in the composition in a total content ranging from 0.01% to 20% by weight, preferably ranging from 1% to 20% by weight, more preferentially ranging from 1% to 10% by weight, relative to the total weight of the composition.

According to a preferred embodiment, the compound(s) ii) are chosen from carbonates, carbonate-generating systems and mixtures thereof, preferably from carbonates.

Bicarbonates and/or Bicarbonate-Generating Systems

The term “bicarbonate-generating system” means a system which generates bicarbonate in situ, for instance carbon dioxide in water or by buffering a carbonate with a mineral or organic acid.

Preferably, the bicarbonate(s) are chosen from:
alkali metal bicarbonates;
alkaline-earth metal bicarbonates;
the compounds of formula N⁺R¹R²R³R⁴ HCO₃⁻ in which R¹, R², R³ and R⁴ represent, independently of each other, a hydrogen atom or a (C₁-C₄)alkyl group optionally substituted with a hydroxyl group;
aminoguanidine bicarbonate;
mixtures thereof.

More preferentially, the bicarbonate(s) are chosen from sodium bicarbonate, potassium bicarbonate, lithium bicarbonate, caesium bicarbonate, calcium bicarbonate, magnesium bicarbonate, ammonium bicarbonate, choline bicarbonate, triethylammonium bicarbonate, aminoguanidine bicarbonate, and mixtures thereof.

Even more preferentially, the bicarbonate(s) are chosen from sodium bicarbonate, potassium bicarbonate, caesium bicarbonate, calcium bicarbonate, magnesium bicarbonate, ammonium bicarbonate, and mixtures thereof.

Most preferentially, the bicarbonate(s) are chosen from sodium bicarbonate, potassium bicarbonate, ammonium bicarbonate and mixtures thereof.

According to a particularly preferred embodiment, the bicarbonate included in the composition is ammonium bicarbonate.

The bicarbonates may originate from a natural water, for example spring water from the Vichy basin or from La Roche Posay or Badoit water.

The bicarbonate(s) and/or the bicarbonate-generating system(s) are preferably present in the composition in a total content ranging from 0.01% to 20% by weight, more preferentially ranging from 1% to 15% by weight, even more preferentially ranging from 2% to 15% by weight, most preferentially ranging from 4% to 15% by weight, relative to the total weight of the composition.

According to a preferred embodiment, the bicarbonate(s) are present in a total content ranging from 0.01% to 20% by weight, preferably ranging from 1% to 15% by weight, more preferentially ranging from 2% to 15% by weight, even more preferentially ranging from 4% to 15% by weight, relative the total weight of the composition.

According to a preferred embodiment, the compound(s) ii) are chosen from bicarbonates, bicarbonate-generating systems and mixtures thereof, preferably from bicarbonates.

### Silicates

The composition according to the invention also comprises iii) one or more silicates.

The silicate(s) are preferably water-soluble.

The term “water-soluble silicate” means a silicate which has a solubility in water at ordinary room temperature (25° C.) and at atmospheric pressure (760 mmHg) of greater than 0.5% by weight, preferably greater than 1% by weight.

Preferably, the silicate(s) are chosen from alkali metal silicates, alkaline-earth metal silicates, aluminium silicates, trimethylammonium silicates and mixtures thereof.

More preferentially, the silicate(s) are chosen from sodium silicates, potassium silicates, calcium silicates, aluminium silicates, trimethylammonium silicates and mixtures thereof. Even more preferentially, the silicate(s) are chosen from sodium silicates.

Preferably, the silicate(s) are chosen from compounds having the INCI name Sodium Silicate (CAS: [1344-09-8]) and/or Sodium Metasilicate (CAS: [6834-92-0]).

The silicate(s) are preferably present in the composition in a total content ranging from 3% to 35% by weight, most preferentially ranging from 4% to 20% by weight, relative to the total weight of the composition.

The weight ratio of the total amount of carbonate(s) and/or carbonate-generating system(s)/total amount of silicate(s) is preferably from 0.00025 to 20, more preferentially from 0.028 to 10 and even more preferentially from 0.028 to 3.4.

According to a preferred embodiment, the weight ratio of the total amount of carbonate(s)/total amount of silicate(s) is from 0.00025 to 20, preferably from 0.028 to 10, more preferentially from 0.028 to 3.4.

The weight ratio of the total amount of carbonate(s) and/or carbonate-generating system(s)/total amount of chemical oxidizing agent(s) is preferably from 0.0008 to 20, more preferentially from 0.1 to 6.6 and even more preferentially from 0.1 to 2.9.

According to a preferred embodiment, the weight ratio of the total amount of carbonate(s)/total amount of chemical oxidizing agent(s) is from 0.0008 to 20, preferably from 0.1 to 6.6 and more preferentially from 0.1 to 2.9.

According to a preferred embodiment, the weight ratio of the total amount of carbonate(s)/total amount of hydrogen peroxide is from 0.0008 to 20, preferably from 0.1 to 6.6 and more preferentially from 0.1 to 2.9.

The weight ratio of the total amount of bicarbonate(s) and/or bicarbonate-generating system(s)/total amount of silicate(s) is preferably from 0.00025 to 20, more preferentially from 0.02 to 7.5 and even more preferentially from 0.05 to 5.

According to a preferred embodiment, the weight ratio of the total amount of bicarbonate(s)/total amount of silicate(s) is from 0.00025 to 20, preferably from 0.02 to 7.5, more preferentially from 0.05 to 5.

The weight ratio of the total amount of bicarbonate(s) and/or bicarbonate-generating system(s)/total amount of chemical oxidizing agent(s) is preferably from 0.0008 to 20, more preferentially from 0.11 to 5 and even more preferentially from 0.2 to 4.2.

According to a preferred embodiment, the weight ratio of the total amount of bicarbonate(s)/total amount of chemical oxidizing agent(s) is from 0.0008 to 20, preferably from 0.11 to 5 and more preferentially from 0.2 to 4.2.

According to a more preferred embodiment, the weight ratio of the total amount of bicarbonate(s)/total amount of hydrogen peroxide is from 0.0008 to 20, preferably from 0.11 to 5 and more preferentially from 0.2 to 4.2.

The weight ratio of the total amount of carbonate(s) and/or carbonate-generating system(s)/total amount of bicarbonate(s) and/or bicarbonate-generating system(s) is preferably from 0.0005 to 2000, more preferentially from 0.06 to 20 and even more preferentially from 0.06 to 5.

According to a preferred embodiment, the weight ratio of the total amount of carbonate(s)/total amount of bicarbonate(s) is from 0.0005 to 2000, preferably from 0.06 to 20 and more preferentially from 0.06 to 5.

### Alcohols

The composition according to the invention also comprises iv) one or more alcohols. Preferably, the alcohol(s) is/are chosen from the compounds of formula (Ia) below, salts thereof, optical isomers thereof, geometrical isomers thereof, tautomers thereof, solvates thereof and mixtures thereof:

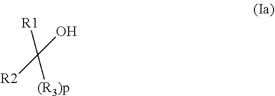

in which formula (Ia):
R¹ and R² independently represent:
a hydrogen atom; or
a linear or branched, saturated or unsaturated hydrocarbon-based group comprising from 1 to 30 carbon atoms, optionally interrupted with one or more non-adjacent oxygen atoms and/or optionally substituted with one or more identical or different groups chosen from: hydroxyl (—OH) or (poly)(hydroxy)alkyl; or
a (hetero)cyclic group optionally substituted with one or more identical or different groups chosen from: —OR″ or hydroxyl (—OH);

R¹ and R² possibly forming together with the carbon atom which bears them a (hetero)cyclic group optionally substituted with one or more identical or different groups chosen from:
a hydroxyl group (—OH);
a (poly)(hydroxy)alkyl group;
a group —OR″;
an alkyl group optionally interrupted with one or more carbonyl groups (CO) and/or optionally substituted with a (hetero)cyclic group, the (hetero)cyclic group itself being optionally substituted with one or more identical or different groups chosen from: hydroxyl (—OH) or (poly)(hydroxy)alkyl;
a group optionally substituted with a (hetero)cyclic group, the (hetero)cyclic group itself being optionally substituted with one or more identical or different groups chosen from: hydroxyl (—OH) or (poly)(hydroxy)alkyl;
R³ represents:
a linear or branched, saturated or unsaturated or aromatic hydrocarbon-based group comprising from 1 to 30 carbon atoms, optionally interrupted with one or more non-adjacent oxygen atoms or one or more carbonyl (CO) groups and/or optionally substituted with one or more identical or different groups chosen from: hydroxyl (—OH) or (poly)(hydroxy)alkyl or —CHO or (hetero)cyclic, the (hetero)cyclic group itself being optionally substituted with one or more identical or different groups chosen from: hydroxyl (—OH) or (poly)(hydroxy)alkyl or —OR″ or —CHO; or
a (hetero)cyclic group optionally substituted with one or more identical or different groups chosen from: —OR″ or (poly)(hydroxy)alkyl or —CHO;
p=0 or 1;
R″ represents:
a linear or branched, saturated or unsaturated hydrocarbon-based group comprising from 1 to 30 carbon atoms, optionally substituted with one or more hydroxyl groups (—OH); or
a (hetero)cyclic group optionally substituted with one or more identical or different groups chosen from: hydroxyl (—OH) or (poly)(hydroxy)alkyl.

Preferably, the alcohol(s) does/do not contain any amino groups (primary amine NH₂, secondary amine or tertiary amine).

More preferentially, the alcohol(s) is/are chosen from the following compounds 1a to 50a, salts thereof, optical isomers thereof, geometrical isomers thereof, tautomers thereof, solvates thereof and mixtures thereof:

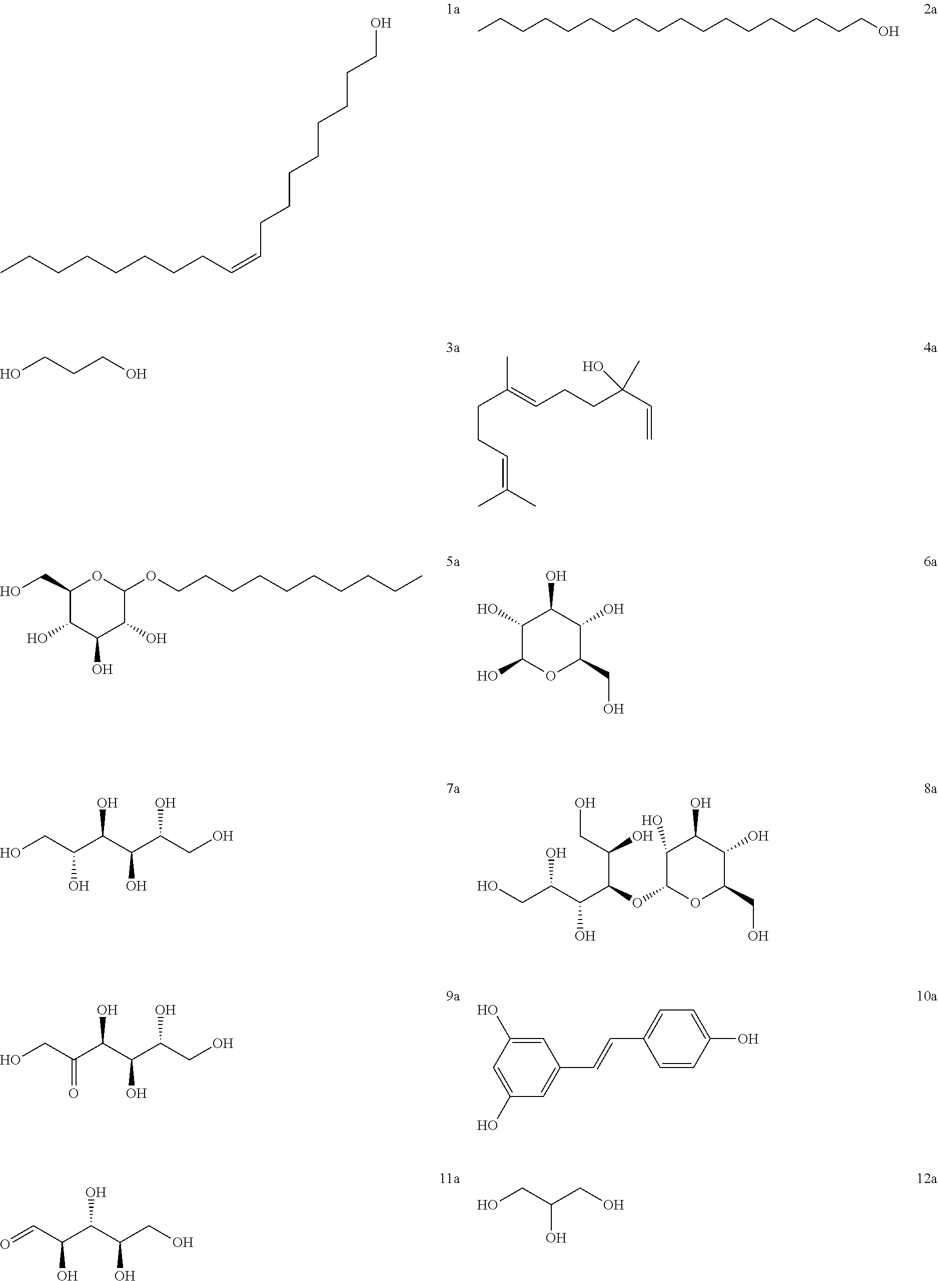

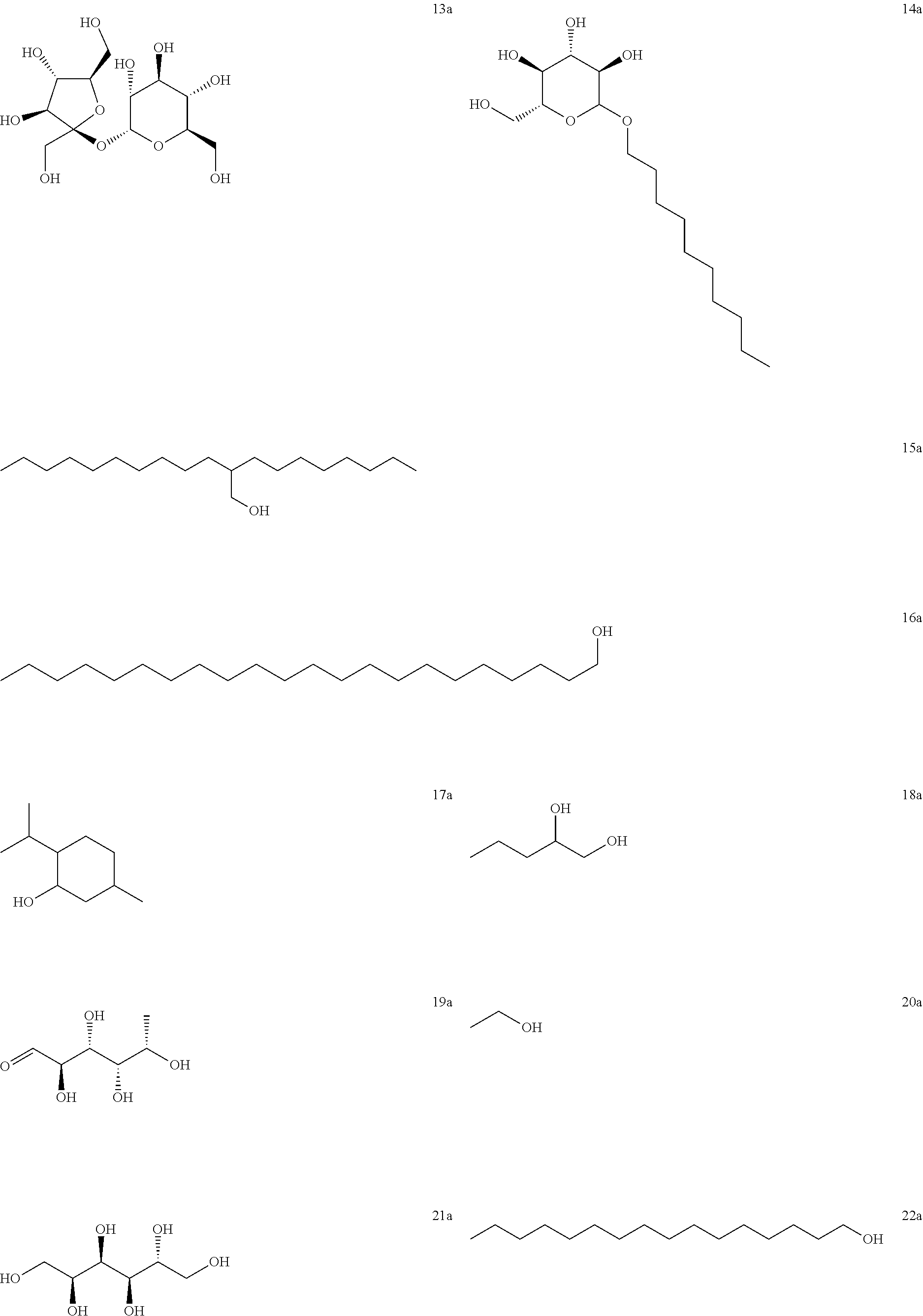

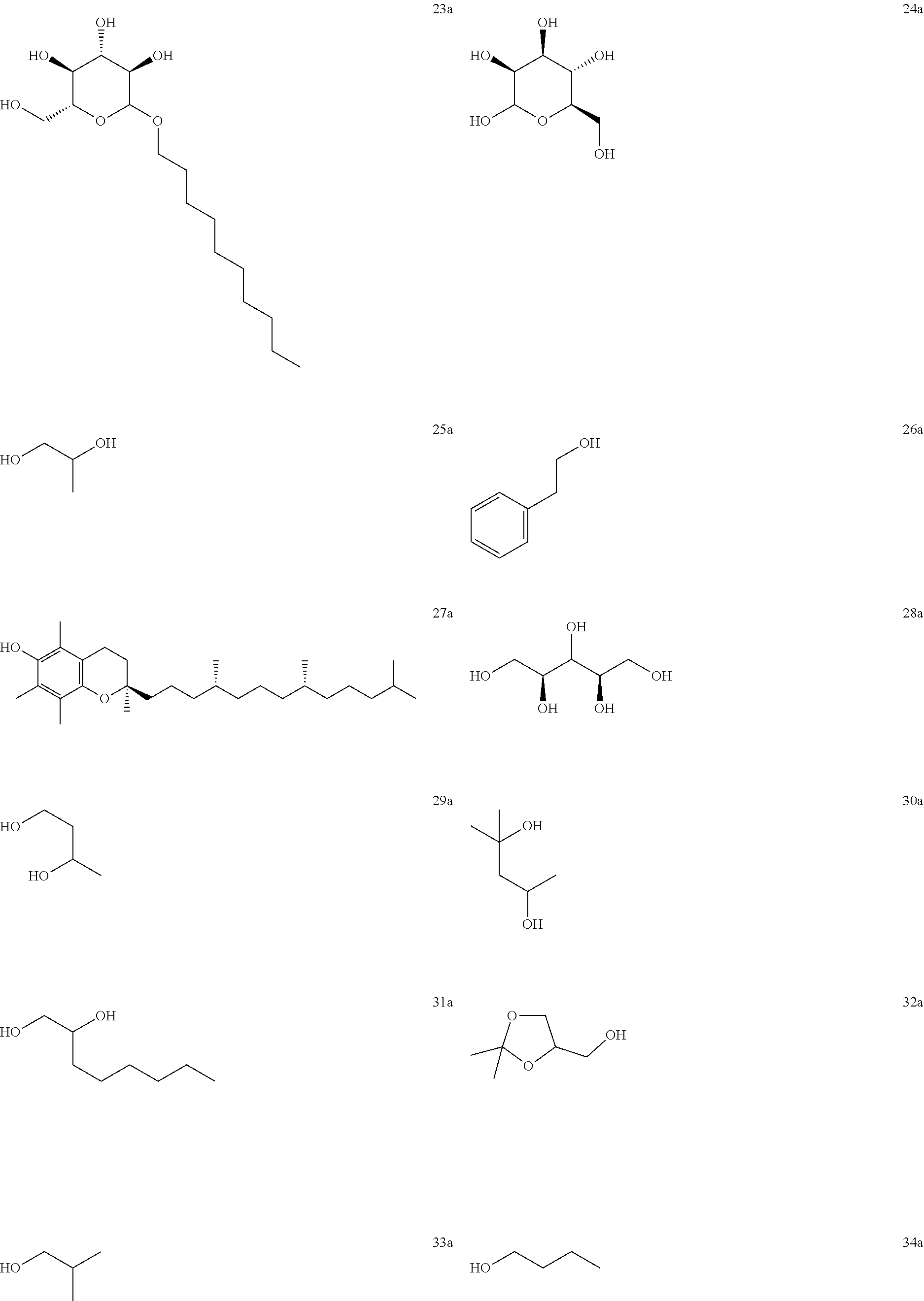

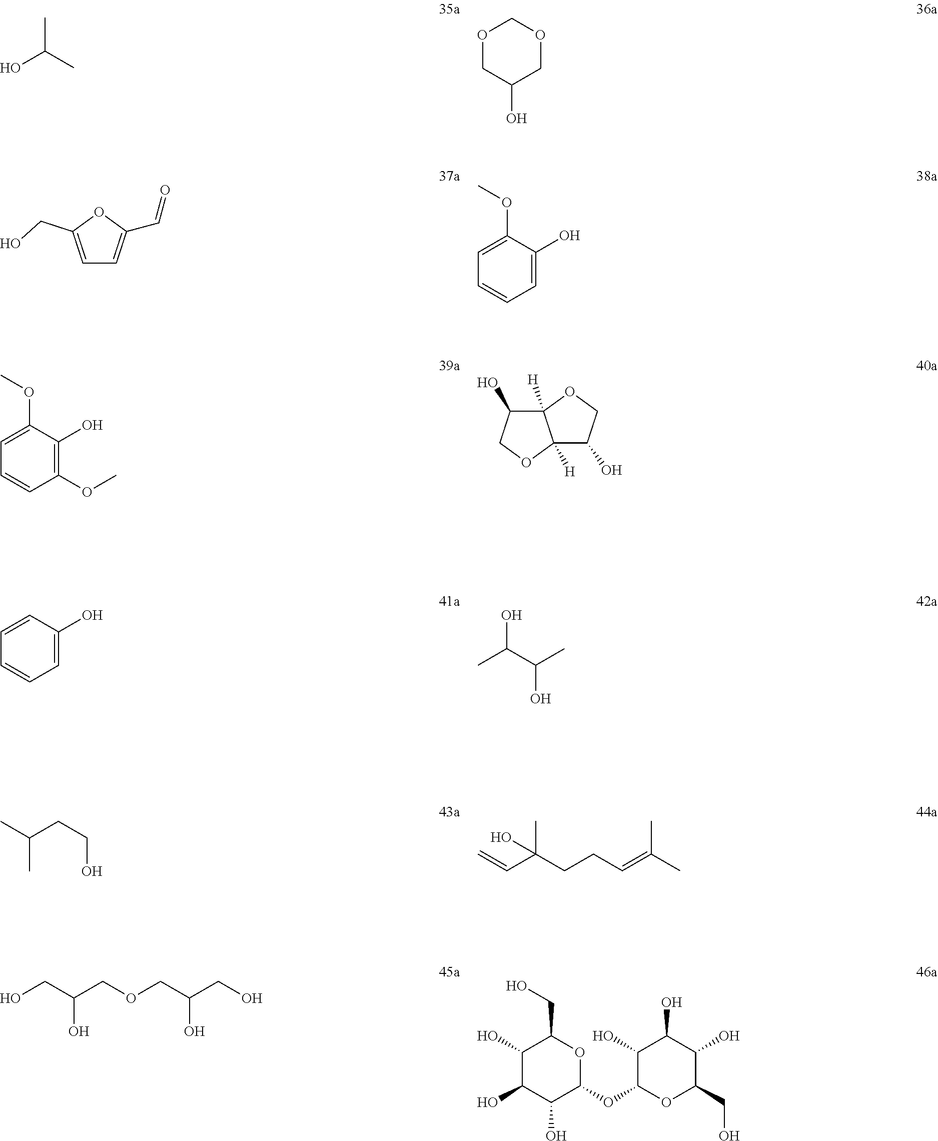

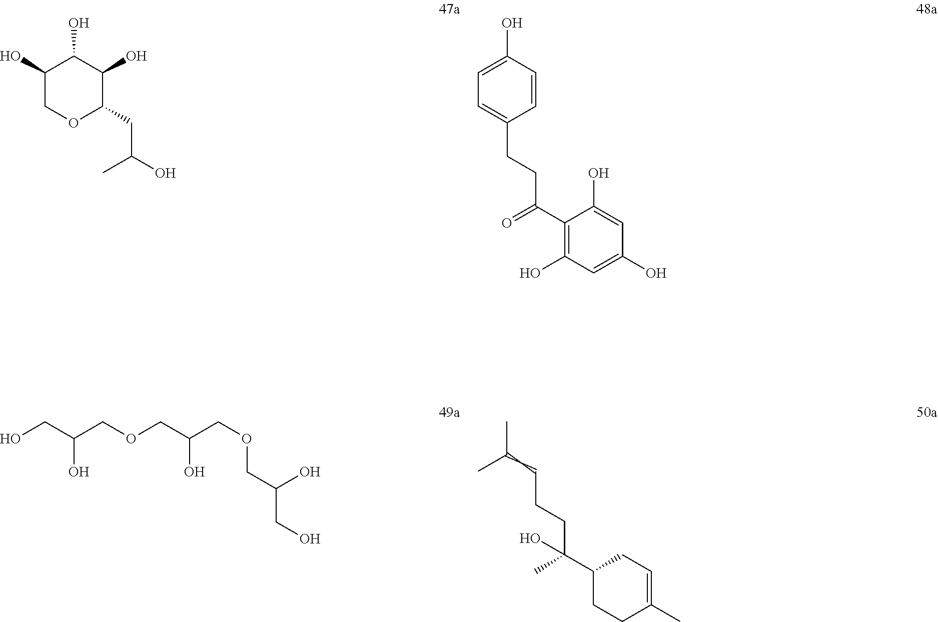 [SMILES: CC(O)C[C@@H]1OC[C@@H](O)[C@H](O)[C@H]1O ; O=C(CCc1ccc(O)cc1)c1c(O)cc(O)cc1O ; OCC(O)COCC(O)COCC(O)CO ; CC(C)=CCC[C@](C)(O)[C@@H]1CC=C(C)CC1]

Even more preferentially, the alcohol(s) is/are chosen from compounds 1a to 29a, 45a, 49a and 50a, salts thereof, optical isomers thereof, geometrical isomers thereof, tautomers thereof, solvates thereof and mixtures thereof.

More preferably, the alcohol(s) is/are chosen from compounds 1a, 2a, 3a, 12a, 15a, 16a, 18a, 21a, 22a, 26a, 29a, 45a and 49a, salts thereof, optical isomers thereof, geometrical isomers thereof, tautomers thereof, solvates thereof and mixtures thereof.

Even more preferably, the alcohol(s) is/are chosen from compounds 1a, 12a, 16a, 18a and 26a, salts thereof, optical isomers thereof, geometrical isomers thereof, tautomers thereof, solvates thereof and mixtures thereof.

According to one form of the invention, the alcohol(s) is/are chosen from the compounds of formula (Ia) in which p=1, salts thereof, optical isomers thereof, geometrical isomers thereof, tautomers thereof, solvates thereof and mixtures thereof.

According to another form of the invention, the alcohol(s) is/are chosen from the compounds of formula (Ia) in which p=0, salts thereof, optical isomers thereof, geometrical isomers thereof, tautomers thereof, solvates thereof and mixtures thereof.

According to a particular variant of the invention, the alcohol(s) is/are chosen from the compounds of formula (Ia), salts thereof, optical isomers thereof, geometrical isomers thereof, tautomers thereof, solvates thereof and mixtures thereof, in which formula (Ia) p=1, R¹ and R² represent a hydrogen atom and R³ represents:
a linear or branched, saturated or unsaturated or aromatic hydrocarbon-based group, preferably linear or branched, and saturated or unsaturated, comprising from 1 to 30 carbon atoms, optionally interrupted with one or more non-adjacent oxygen atoms or one or more carbonyl (CO) groups, preferably uninterrupted, and/or optionally substituted with one or more identical or different groups chosen from hydroxyl (OH), —CHO or (hetero)cyclic groups such as phenyl, the (hetero)cyclic group itself being optionally substituted with one or more identical or different groups chosen from OH, —OR″ or —CHO; or
a (hetero)cyclic group optionally substituted with one or more identical or different groups chosen from: OH, —OR″ or (poly)(hydroxy)alkyl or —CHO; R″ being as defined previously.

As examples of alcohols of formula (Ia) according to this variant, mention may be made of the compounds 1a, 2a, 3a, 6a, 7a, 8a, 9a, 11a, 12a, 13a, 14a, 15a, 16a, 18a, 19a, 20a, 21a, 22a, 23a, 24a, 25a, 26a, 28a, 29a, 31a, 32a, 33a, 34a, 37a, 43a, 45a, 46a, 49a, salts thereof, optical isomers thereof, geometrical isomers thereof, tautomers thereof, solvates thereof and mixtures thereof.

According to a preferred variant, the alcohol(s) is/are chosen from the compounds of formula (Ia), salts thereof, optical isomers thereof, geometrical isomers thereof, tautomers thereof, solvates thereof and mixtures thereof, in which formula (Ia) p=1, R¹ and R² represent a hydrogen atom and R³ represents a linear or branched, saturated or unsaturated hydrocarbon-based group comprising from 1 to 30 carbon atoms, optionally interrupted with one or more non-adjacent oxygen atoms or one or more carbonyl groups (CO), preferably uninterrupted, and/or optionally substituted with one or more hydroxyl groups (OH).

According to a particular embodiment, the alcohol(s) is/are chosen from the compounds of formula (Ia), salts thereof, optical isomers thereof, geometrical isomers thereof, tautomers thereof, solvates thereof and mixtures thereof, in which formula (Ia) p=1, R¹ and R² represent a hydrogen atom and R³ represents a (hetero)cyclic group, preferably of 5 or 6 chain members, more preferentially a pyranose ring or a furanose ring optionally substituted with one or more identical or different groups chosen from: OH, —OR″ or (poly)(hydroxy)alkyl or —CHO; R″ being as defined previously.

According to another particular embodiment, the alcohol(s) denotes/denote a sugar or a sugar derivative.

For the purposes of the present invention, the term “sugar” means a monosaccharide or polysaccharide sugar comprising from 2 to 5 saccharide unit(s), preferably from 2 to 3 saccharide unit(s), more preferentially a sugar denotes a compound comprising 1 or 2 saccharide unit(s) (monosaccharide or disaccharide).

It is understood that for the sugars, when the sugar represents a monosaccharide, then it may be in pyranose form (the sugar heterocycle of which it is composed has 6 chain members) or furanose form (the sugar heterocycle of which it is composed has 5 chain members); and when the sugar represents a polysaccharide radical, it comprises a sequence of 2 to 5 saccharide units which may be identical to or different from each other and which may be in furanose or pyranose form. Preferably, the polysaccharide is a disaccharide resulting from the linking of a saccharide unit in furanose form and a unit in pyranose form or the linking of a saccharide unit in pyranose form and a unit in furanose form. Whether monosaccharides or polysaccharides, each saccharide unit may be in the L laevorotatory or D dextrorotatory form, and in the α or β anomeric form.

Preferably, the monosaccharide sugars are chosen from: glucose, galactose, mannose, xylose, lyxose, fucose, arabinose, rhamnose, quinovose, fructose, sorbose, talose, and mixtures thereof. Preferably, the disaccharide(s) are chosen from: lactose, maltulose, palatinose, lactulose, amygdalose, turanose, cellobiose, isomaltose, rutinose, maltose, and mixtures thereof.

For the purposes of the present invention, the term “sugar derivative” means compounds in which one or more of the hydroxyl radicals (OH) borne by the furanose or pyranose ring are replaced with one or more alkoxy radicals OR″ with R″ as defined previously. As an example of a sugar derivative, mention may be made of compound 13a.

According to another particular embodiment, the alcohol(s) is/are chosen from the compounds of formula (Ia) such that p=0, salts thereof, optical isomers thereof, geometrical isomers thereof, tautomers thereof, solvates thereof and mixtures thereof, and R¹ and R², together with the carbon atom which bears them, form an aromatic or non-aromatic, preferably 6-membered (hetero)cyclic group, optionally substituted with one or more identical or different groups chosen from:
a hydroxyl group (—OH);
a (poly)(hydroxy)alkyl group;
a group —OR″ with R″ as defined previously;
an alkyl or alkenyl group optionally interrupted with one or more carbonyl (CO) groups, which is preferably uninterrupted, and/or optionally substituted with a (hetero)cyclic group, the (hetero)cyclic group itself being optionally substituted with one or more identical or different groups chosen from: hydroxyl (—OH) or (poly)(hydroxy)alkyl; more preferentially, the alcohols denote a saturated or aromatic 5- or 6-membered ring bearing at least one hydroxyl (—OH) group, preferably a hydroxyl (—OH) group, and optionally substituted with one or more groups —OR″ with R″ as defined previously.

As examples of alcohols of formula (Ia) according to this variant, mention may be made of compounds 17a, 38a, 39a and 41a.

The alcohol(s) iv) are preferably present in the composition in a total content ranging from 0.01% to 50% by weight, more preferentially ranging from 1% to 30% by weight, even more preferentially ranging from 1% to 15% by weight, relative to the total weight of the composition.

Compounds iv′)

The composition according to the invention also comprises iv′) one or more compounds chosen from:
a) organic acids;
b) amphoteric or zwitterionic compounds chosen from: phospholipids such as lecithins, amino acids, amphoteric or zwitterionic surfactants and mixtures thereof;
c) esters, amides, imines and mixtures thereof;
d) organic amines;
e) compounds consisting of a saturated or unsaturated, linear or branched hydrocarbon-based chain comprising from 1 to 30 carbon atoms, the hydrocarbon-based chain being optionally substituted with one or more cyclic groups and/or optionally interrupted with one or more non-adjacent oxygen atoms; saturated or unsaturated, optionally aromatic, monocyclic or polycyclic, condensed or non-condensed carbocyclic compounds comprising from 3 to 22 carbon atoms, optionally substituted with one or more identical or different groups chosen from: (C₁-C₆)alkyl or (C₁-C₆)alkenyl or (C₁-C₆)alkoxy and/or optionally interrupted with one or more carbonyl groups (CO); monocyclic or polycyclic, condensed or non-condensed, saturated or unsaturated, notably aromatic, heterocyclic compounds comprising from 5 to 22 members and containing from 1 to 3 oxygen atoms, optionally substituted with one or more identical or different groups chosen from: (C₁-C₆)alkyl or (C₁-C₆)alkoxy and/or optionally interrupted with one or more carbonyl groups (CO) and mixtures thereof;
f) and mixtures thereof.

### Organic Acids a)

Preferably, the organic acids are chosen from the compounds of formulae (I) and/or (II) below, salts thereof, optical isomers thereof, geometrical isomers thereof, tautomers thereof, solvates thereof and mixtures thereof:

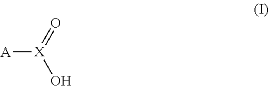

in which formula (I):
X represents a carbon atom or S═O or P—OH, preferably a carbon atom;
A represents a group —OR⁴ or a group —CR¹(R²)p(R³), it being understood that if X represents P—OH, then A represents a group —OR⁴;
p=0 or 1;
R¹ represents a hydrogen atom or a hydroxyl group (—OH) or a group —NR^{a}—R^{c} or a group —NR^{a}—CO—R^{c};
R² represents a hydrogen atom or a hydroxyl group (—OH) or a group —X(O)OH with X as defined previously, in particular with X representing a carbon atom;
R³ represents a hydrogen atom or a hydroxyl group (—OH) or a (hetero)cyclic group optionally substituted with one or more identical or different groups chosen from (C₁-C₆)alkyl, notably (C₁-C₄)alkyl such as methyl, alkylcarbonyl —(CO)—R^{c}, alkylcarbonyloxy —O—CO—R^{c}, alkyloxycarbonyl —CO—O—R^{c}, —OH, (C₁-C₆)alkoxy, in particular (C₁-C₄)alkoxy such as methoxy, —X(O)OH with X as defined previously, in particular with X representing a carbon atom or —O—X(O)OH with X as defined previously or R³ represents an alkyl group or an alkenyl group or an alkylamino group or an alkylamido group, said alkyl or alkenyl or alkylamino or alkylamido group being:
optionally substituted with one or more identical or different groups chosen from a group —X(O)OH with X as defined previously, in particular with X representing a carbon atom; a hydroxyl group (—OH) or a (hetero)cyclic group; and/or
optionally interrupted with one or more heteroatoms or groups chosen from —O—, —NR^{a}—, —S—, —CO—, or combinations thereof such as —O—CO—, —(CO)—O—, —NR^{a}—(CO)— or —(CO)—NR^{a}—;

R¹ and R³ possibly forming together with the carbon atom which bears them a (hetero)cyclic group optionally substituted with one or more identical or different groups chosen from: (C₁-C₆)alkyl, notably (C₁-C₄)alkyl such as methyl, alkylcarbonyl —(CO)—R^{c}, alkylcarbonyloxy —O—CO—R^{c}, alkyloxycarbonyl —CO—O—R^{c}, —OH, (C₁-C₆)alkoxy, notably (C₁-C₄)alkoxy such as methoxy, —X(O)OH with X as defined previously, in particular with X representing a carbon atom or —O—X(O)OH with X as defined previously, in particular with X═P—OH;

R¹ and R³ possibly forming together with the carbon atom bearing them a carbonyl group —CO—;
R⁴ represents a hydrogen atom or an alkyl group or an alkenyl group or a (hetero)cyclic group, said alkyl or alkenyl or (hetero)cyclic group being optionally substituted with one or more identical or different groups chosen from: a group —X(O)OH with X as defined previously, in particular with X representing a carbon atom or P—OH; a hydroxyl group (—OH) or a (hetero)cyclic group;
R^{a} represents a hydrogen atom or a (C₁-C₄)alkyl group optionally substituted with one or more groups —X(O)OH with X as defined previously, in particular with X representing a carbon atom;
R^{c} represents an alkyl group or an alkenyl group or an alkylamino group or an alkylamido group, said alkyl or alkenyl or alkylamino or alkylamido group being:
optionally substituted with one or more identical or different groups chosen from: a group —X(O)OH with X as defined previously, in particular with X representing a carbon atom; a hydroxyl group (—OH) or a (hetero)cyclic group; and/or
optionally interrupted with one or more heteroatoms or groups chosen from —O—, —S—, —CO—, —O—CO—, —O—CO—, —NR^{a}—(CO)— or —(CO)—NR^{a}—;

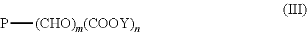

in which formula (II):
P represents a polysaccharide chain constituted of monosaccharides comprising 5 carbon atoms or more than 5 carbon atoms, preferably 6 or more than 6 carbon atoms and more particularly 6 carbon atoms;
Y is chosen from a hydrogen atom, the ions derived from an alkali metal or an alkaline-earth metal such as sodium or potassium, ammonia, organic amines such as monoethanolamine, diethanolamine, triethanolamine and 3-amino-1,2-propanediol and basic amino acids such as lysine, arginine, sarcosine, ornithine and citrulline;
m+n is greater than or equal to 1;
m is such that the degree of substitution of the polysaccharide with one or more aldehyde groups (DS(CHO)) is within the range from 0.001 to 2 and preferably from 0.005 to 1.5;
n is such that the degree of substitution of the polysaccharide with one or more carboxylic groups (DS(COOX)) is within the range from 0 to 2 and preferably from 0.001 to 1.5.

The expression “degree of substitution DS(CHO) or DS(COOX) of the polysaccharides according to the invention” means the ratio between the number of carbons oxidized to give an aldehyde or carboxylic group for all the repeating units and the number of elementary monosaccharides (even opened by preoxidation) constituting the polysaccharide.

The groups CHO and COOX may be obtained during the oxidation of certain carbon atoms, for example in position C2, C3 or C6, of a saccharide unit comprising 6 carbon atoms. Preferably, the oxidation may take place at C2 and at C3, more particularly from 0.01% to 75% by number and preferably from 0.1% to 50% by number of the rings having possibly been opened.

The polysaccharide chain, represented by P, is preferably chosen from inulins, celluloses, starches, guar gums, xanthan gums, pullulan gums, alginate gums, agar-agar gums, carrageenan gums, gellan gums, acacia gums, xyloses and tragacanth gums, and derivatives thereof, cellobiose, maltodextrin, scleroglucan, chitosan, ulvan, fucoidan, alginate, pectin, heparin, hyaluronic acid or mixtures thereof, more preferentially chosen from inulins or starches. Even more preferentially, the polysaccharide chain is inulin.

The term “derivative” means the compounds obtained by chemical modification of the mentioned compounds. They may be esters, amides or ethers of said compounds.

The oxidation may take place according to a process known in the art, for example according to the process described in FR 2 842 200, in document FR 2 854 161 or in the article “Hydrophobic films from maize bran hemicelluloses” by E. Fredon et al., Carbohydrate Polymers 49, 2002, pages 1 to 12. Another oxidation process is described in the article “Water soluble oxidized starches by peroxide reaction extrusion” Industrial Crops and Products 75 (1997) 45-52—R. E. Wing, J. L. Willet. These oxidation processes are easy to perform, are efficient and do not generate any toxic by-products or by-products that are difficult to remove.

The peroxides that may be used in these oxidation processes may be an alkali metal or alkaline-earth metal percarbonate or perborate, an alkyl peroxide, peracetic acid or hydrogen peroxide. Hydrogen peroxide is particularly preferred, in so far as it is readily accessible and does not produce interfering by-products.

The amount of peroxide in the reaction medium is between 0.05 and 1 molar equivalent per glucose unit of the polysaccharide, preferably between 0.1 and 0.8 molar equivalent. It is preferable to add the peroxide in successive portions, leaving the reaction medium stirring between two additions.

A single phthalocyanin or a mixture of phthalocyanins, for example a mixture of cobalt phthalocyanin and of iron phthalocyanin, may be used as catalyst in the oxidation process. The amount of catalyst depends on the desired degree of substitution. In general, a small amount, for example an amount corresponding to 0.003 to 0.016 molar equivalent per 100 glucose units of polysaccharide, is suitable.

The process may also be performed by placing the polysaccharide in pulverulent form in contact with the catalyst dissolved in a small volume of water and with the peroxide. This process is referred to as a “semi-dry” process.

The process may be performed by reactive extrusion in the presence of peroxide.

More preferentially, the polysaccharide is obtained by oxidation of inulin, cellulose, carboxymethylcellulose, hydroxyethylcellulose, hydroxypropylcellulose, hydroxypropylmethylcellulose, methylcellulose, starch, starch acetate, hydroxyethyl starch, hydroxypropyl starch, guar gum, carboxymethyl guar gum, carboxymethylhydroxypropyl guar gum, hydroxyethyl guar gum, hydroxypropyl guar gum, xylose or xanthan gum, carrageenan gum, cellobiose, maltodextrin, scleroglucan, chitosan, ulvan, fucoidan, alginate, pectin, heparin and hyaluronic acid, or mixtures thereof.

Preferentially, the polysaccharide is obtained by oxidation of inulin or starch, more preferentially by oxidation of inulin.

According to one embodiment, the polysaccharide is obtained by oxidation of inulin by performing a reactive extrusion process in the presence of hydrogen peroxide.

The polysaccharide chain before and after oxidation preferably has a weight-average molecular mass ranging from 400 to 15 000 000, better still from 500 to 10 000 000 and more particularly from 500 to 50 000 g/mol.

The polysaccharides that are most particularly preferred in the invention are those corresponding to formula (II) in which: P represents a polymer chain derived from inulin or from starch; m is such that the degree of substitution of the polysaccharide with one or more aldehyde groups (DS(CHO)) is within the range from 0.005 to 2.5; n is such that the degree of substitution of the polysaccharide with one or more carboxylic groups (DS(COOX)) is within the range from 0.001 to 2.

Even more preferably, the radical P of formula (II) as defined previously represents a polymer chain derived from inulin; m is such that the degree of substitution of the polysaccharide with one or more aldehyde groups (DS(CHO)) is within the range from 0.01 to 1; n is such that the degree of substitution of the polysaccharide with one or more carboxylic groups (DS(COOX)) is within the range from 0.01 to 2.

More preferentially, the organic acids are chosen from the compounds of formula (I) as defined previously, salts thereof, optical isomers thereof, geometrical isomers thereof, tautomers thereof, solvates thereof and mixtures thereof.

Even more preferentially, the organic acids are chosen from compounds 1 to 40 below, salts thereof, optical isomers thereof, geometrical isomers thereof, tautomers thereof, solvates thereof and mixtures thereof:

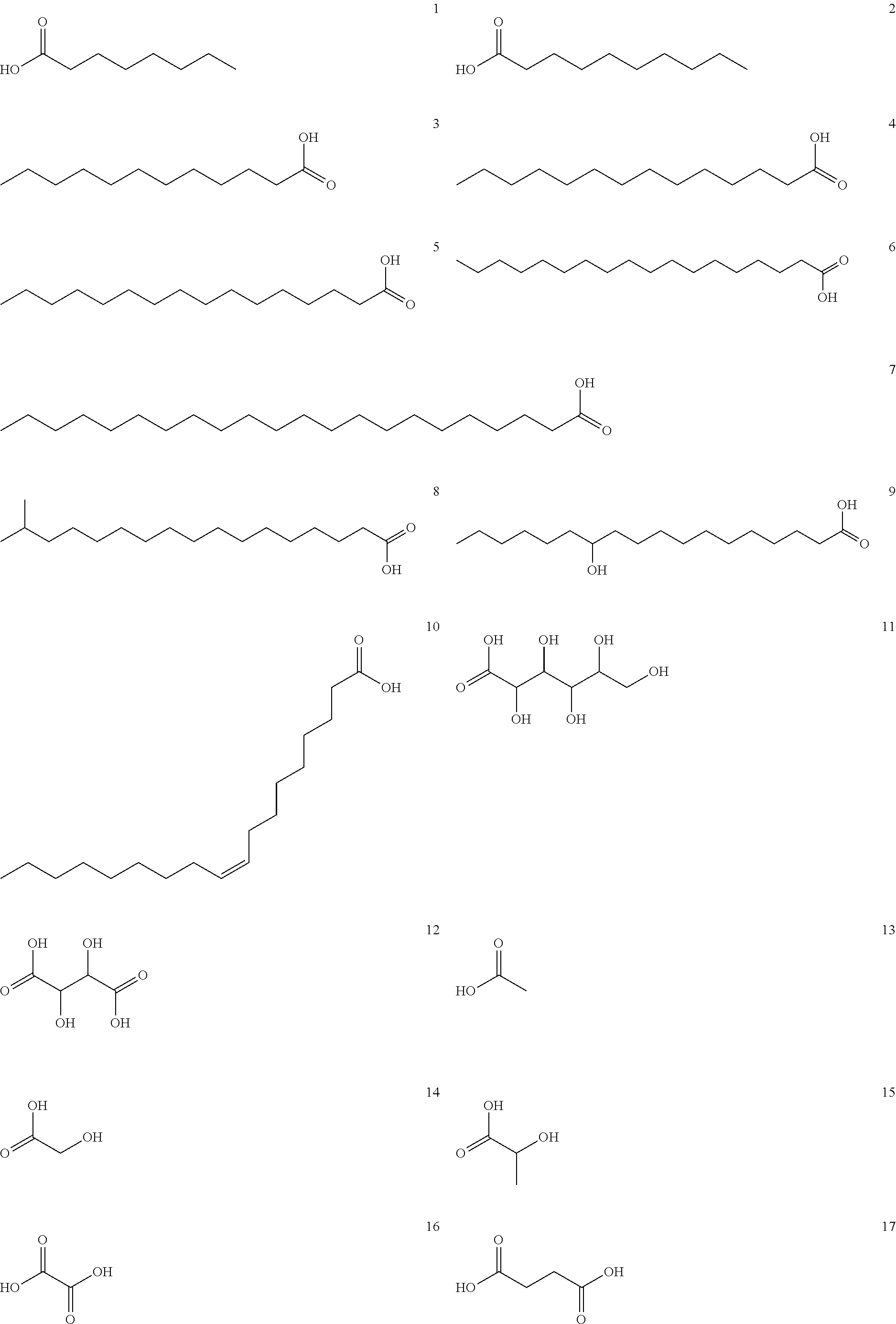

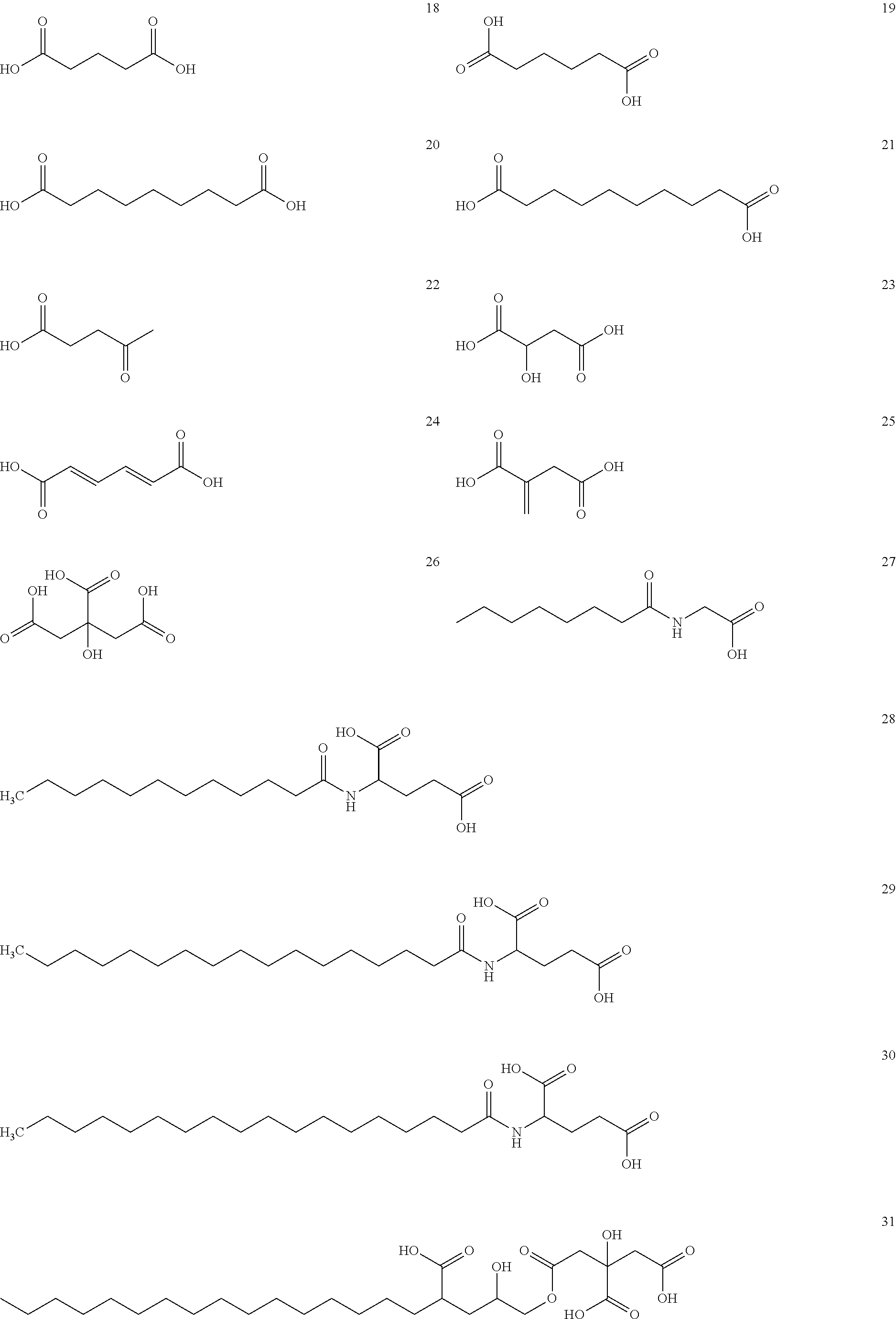

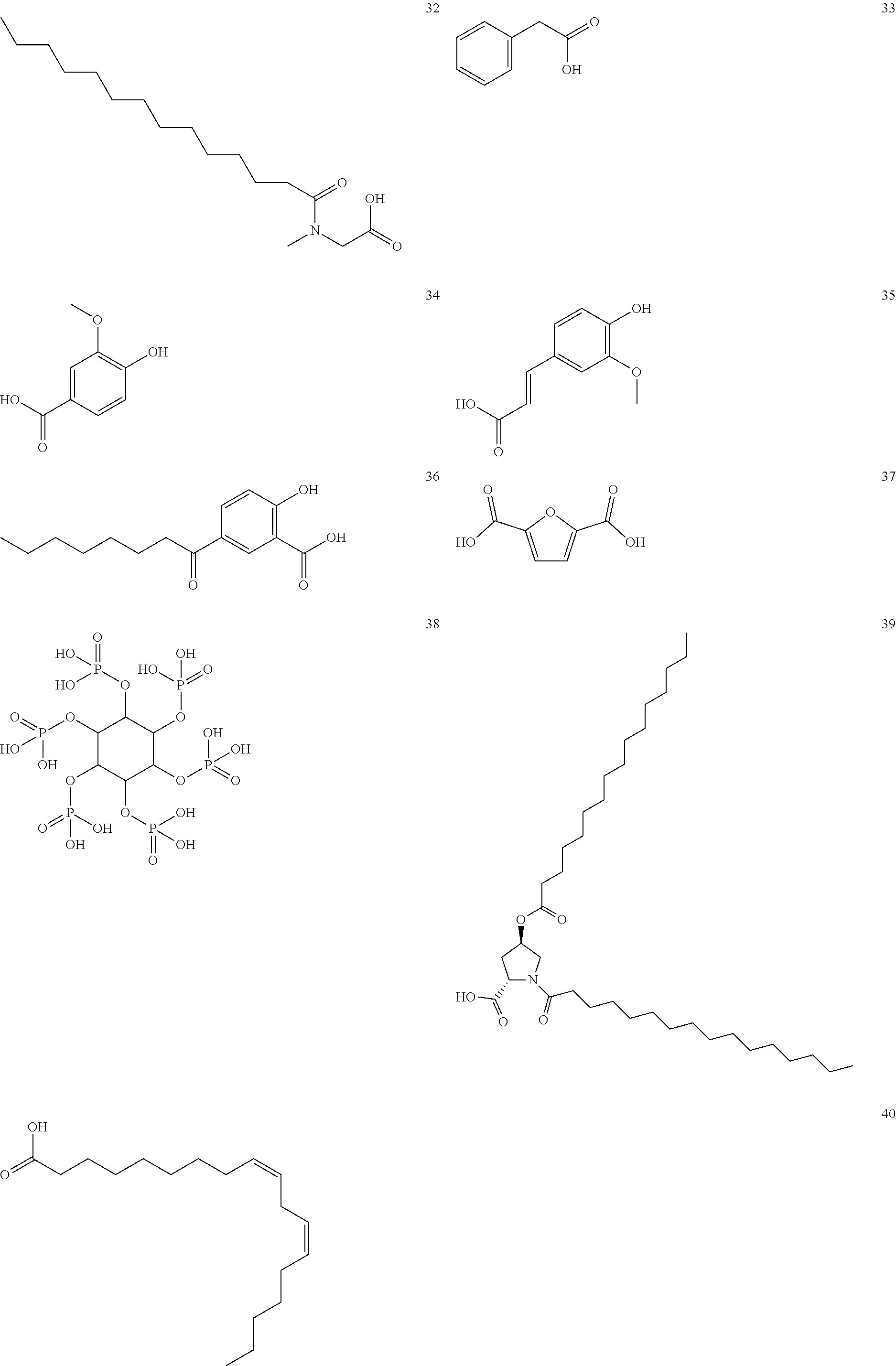

More preferably, the organic acids are chosen from compounds 1, 2, 6, 7, 8, 9, 10, 15, 18, 20, 22, 23, 26, 28 and 31, salts thereof, optical isomers thereof, geometrical isomers thereof, tautomers thereof, solvates thereof and mixtures thereof.

Even more preferably, the organic acids are chosen from compounds 1, 2, 20, 26, 28 and 31, salts thereof, optical isomers thereof, geometrical isomers thereof, tautomers thereof, solvates thereof and mixtures thereof.

Better still, the organic acids are chosen from compounds 26 and 31, salts thereof, optical isomers thereof, geometrical isomers thereof, tautomers thereof, solvates thereof and mixtures thereof:

According to a preferred embodiment of the invention, the organic acids are chosen from compounds of formula (I), salts thereof, optical isomers thereof, geometrical isomers thereof, tautomers thereof, solvates thereof and mixtures thereof, in which formula (I) X represents a carbon atom, A represents a group —CR¹(R²)p(R³) in which p=1, R¹ represents a hydrogen atom or a group —NR^{a}—CO—R^{c} with R^{a} and R^{c} as defined previously, preferably R^{a} denotes a hydrogen atom and R^{c} denotes an alkyl radical, R² represents a hydrogen atom or a hydroxyl group (—OH), R³ represents a (hetero)cyclic group optionally substituted with one or more identical or different groups chosen from (C₁-C₆)alkyl, notably (C₁-C₄)alkyl such as methyl, alkylcarbonyl —(CO)—R^{c}, alkylcarbonyloxy —O—CO—R^{c}, alkyloxycarbonyl —CO—O—R^{c}, —OH, (C₁-C₆)alkoxy, notably (C₁-C₄)alkoxy such as methoxy or —C(O)OH or R³ represents an alkyl group or an alkenyl group, said alkyl or alkenyl group being:
optionally substituted with one or more identical or different groups chosen from a —C(O)OH group; a hydroxyl group (—OH); and/or
optionally interrupted with —CO—, —O—CO—, —(CO)—O—, —NR^{a}—(CO)—, —(CO)—NR^{a}— with R^{a} as defined previously, preferably optionally interrupted with —CO—, —O—CO—, —(CO)—O—.

According to a particular embodiment, the organic acids are chosen from the compounds of formula (I), salts thereof, optical isomers thereof, geometrical isomers thereof, tautomers thereof, solvates thereof and mixtures thereof, in which formula (I) X represents a carbon atom, A represents a group —CR¹(R²)(R³) in which R¹ represents a hydrogen atom, R² represents a hydrogen atom or a hydroxyl group (—OH), R³ represents a (hetero)cyclic group, preferably an aromatic hydrocarbon-based group such as phenyl, said (hetero)cyclic group being optionally substituted with one or more identical or different groups chosen from: (C₁-C₆)alkyl, notably (C₁-C₄)alkyl such as methyl, (C₁-C₆)alkoxy, notably (C₁-C₄)alkoxy such as methoxy or —C(O)OH or R³ represents an alkyl group or an alkenyl group optionally interrupted with —NR^{a}—(CO)— or —(CO)—NR^{a}— with R^{a} as defined previously, preferably uninterrupted, and/or optionally substituted with a hydroxyl group. According to this embodiment, the organic acids are preferably chosen from compounds 1, 2, 3, 4, 5, 6, 7, 8, 9, 10, 11, 12, 14, 15, 32, salts thereof, optical isomers thereof, geometrical isomers thereof, tautomers thereof, solvates thereof and mixtures thereof, more preferentially chosen from compounds 1, 2, 3, 4, 5, 6, 7, 8, 9, 10, 11, 12, 14, 15, salts thereof, optical isomers thereof, geometrical isomers thereof, tautomers thereof, solvates thereof and mixtures thereof, even more particularly chosen from compounds 1, 2, 6, 7, 8, 9, 10, 15, salts thereof, optical isomers thereof, solvates thereof and mixtures thereof.

According to another preferred embodiment of the invention, the organic acids are chosen from the compounds of formula (I), salts thereof, optical isomers thereof, geometrical isomers thereof, tautomers thereof, solvates thereof and mixtures thereof, in which formula (I) X represents a carbon atom, A represents a group —CR¹(R²)(R³) in which R¹ represents a hydrogen atom or a group —NR^{a}—CO—R^{c} with R^{a} and R^{c} as defined previously, preferably R^{a} represents a hydrogen atom and R^{c} represents an alkyl radical, R² represents a hydrogen atom or a hydroxyl group (—OH), R³ represents an alkyl group or an alkenyl group, said alkyl or alkenyl group being substituted with one or more —C(O)OH groups, notably substituted with one or two —C(O)OH groups and:
optionally substituted with one or more hydroxyl groups (—OH); and/or
optionally interrupted with —CO—, —O—CO—, —(CO)—O—, —(CO)—NR^{a}—, with R^{a} as defined previously.

According to this embodiment, the organic acids are preferably chosen from compounds 17, 18, 19, 20, 21, 22, 23, 24, 25, 26, 27, 28, 29, 30, 31, 32, salts thereof, optical isomers thereof, geometrical isomers thereof, tautomers thereof, solvates thereof and mixtures thereof, more preferentially chosen from compounds 17, 18, 19, 20, 21, 22, 23, 24, 25, 26, 27, 28, 29, 30, 31, salts thereof, optical isomers thereof, geometrical isomers thereof, tautomers thereof, solvates thereof and mixtures thereof, even more preferentially chosen from compounds 18, 20, 22, 23, 26, 28, 31, most preferentially chosen from compounds 20, 26, 31, salts thereof, optical isomers thereof, geometrical isomers thereof, tautomers thereof, solvates thereof and mixtures thereof.

According to another embodiment of the invention, the organic acids are chosen from the compounds of formula (I), salts thereof, optical isomers thereof, geometrical isomers thereof, tautomers thereof, solvates thereof and mixtures thereof, in which formula (I) X represents a carbon atom, A represents a group —CR¹(R³) in which R¹ and R³, together with the carbon atom which bears them, form a (hetero)cyclic group, preferably an aromatic group, optionally substituted with one or more identical or different groups chosen from: (C₁-C₆)alkyl, notably (C₁-C₄)alkyl such as methyl, alkylcarbonyl —(CO)—R^{c}, alkylcarbonyloxy —O—CO—R^{c}, alkyloxycarbonyl —CO—O—R^{c}, —OH, (C₁-C₆)alkoxy, notably (C₁-C₄)alkoxy such as methoxy, —C(O)OH, with R^{c} as defined previously. According to this embodiment, the organic acids are preferably chosen from compounds 34, 36, 37, salts thereof, optical isomers thereof, geometrical isomers thereof, tautomers thereof, solvates thereof and mixtures thereof.

When they are present in the composition, the organic acids are preferably present in the composition in a content ranging from 0.01% to 50%, more preferentially ranging from 0.1% to 30%, even more preferentially ranging from 0.2% to 15% by weight relative to the total weight of the composition.

### Amphoteric or Zwitterionic Compounds b)

The amphoteric or zwitterionic compounds are chosen from: phospholipids such as lecithins, amino acids, amphoteric or zwitterionic surfactants, salts thereof, optical isomers thereof, geometrical isomers thereof, tautomers thereof, solvates thereof and mixtures thereof.

### Phospholipids

The phospholipids may be of plant or animal origin and may be present in pure form or as a mixture.

Preferably, the phospholipids are phospholipids of plant origin.

The phospholipids are preferably chosen from lecithins.

The lecithins that may be used according to the present invention may be hydrogenated or non-hydrogenated.

Non-hydrogenated lecithins are generally obtained by lipid extraction, using apolar solvents, from plant or animal fatty substances. This lipid fraction usually predominantly comprises glycerophospholipids, including phosphatidylcholine.

The animal or plant sources that may be used to extract non-hydrogenated lecithins are, for example, soybean, sunflower or eggs. The glycerophospholipids included, in high proportion, in these lecithins are mainly phosphatidylcholine and phosphatidylethanolamine.

Preferably, the lecithins are lecithins of plant origin.

The non-hydrogenated lecithins that are suitable for use in the present invention may be lecithins derived from soybean, from sunflower or from egg and/or mixtures thereof.

The lecithins are usually provided in a form dissolved in fatty acids, triglycerides or other solvents, or in the form of powders or cakes.

They are usually mixtures of lecithins, in which the content of glycerophospholipids, in the products as sold, generally ranges from about at least 15% to about at least 95%. Among the non-hydrogenated lecithins that may be suitable for use in the cosmetic compositions in accordance with the present invention, mention may be made of the lecithins sold under the trade names Nattermann Phospholipid®, Phospholipon 80® and Phosale 75® by the company American Lecithin Company, Epikuron 145V, Topcithin 300, Emulmetik 930, Ovothin 200 and Organic Lecithin sold by the company Lucas Meyer, Lipoid S 20 sold by the company Lipoid Kosmetik, and Alcolec F 100 by the company American Lecithin Company.

The phospholipids may be chosen from hydrogenated lecithins.

These hydrogenated lecithins are obtained by controlled hydrogenation of the nonhydrogenated lecithins as described above.

As hydrogenated lecithins that may be used in the composition according to the invention, an example that may be mentioned is the product sold under the reference Nikkol Lecinol S 10 by Nikko.

### Amino Acids

The amino acids may be chosen from acidic amino acids, basic amino acids, polar neutral amino acids and apolar neutral amino acids, and mixtures thereof. The amino acids may be in D or L form or in a mixture.

The term “acidic amino acid” means an amino acid including more acidic functions than basic functions, in particular protein-forming amino acids, the side chain of which includes an acidic function. As examples of acidic amino acids, mention may be made of aspartic acid, glutamic acid and mixtures thereof.

The term “basic amino acid” means an amino acid including more basic functions than acidic functions, in particular protein-forming amino acids, the side chain of which includes a basic function. As examples of basic amino acids, mention may be made of arginine, histidine, lysine and mixtures thereof.

The term “polar neutral amino acid” means an amino acid which is uncharged at pH=7 and including a polar chemical function such as a thiol, alcohol or phenol, or basic amide CONH₂ function. As examples of polar neutral amino acids, mention may be made of serine, threonine, cysteine, asparagine, glutamine, tyrosine and mixtures thereof.

The term “apolar neutral amino acid” means an amino acid which is uncharged at pH=7 and apolar. As examples of apolar neutral amino acids, mention may be made of amino acids chosen from glycine, alanine, valine, leucine, isoleucine, methionine, phenylalanine, tryptophan, proline and mixtures thereof.

### Amphoteric or Zwitterionic Surfactants

The amphoteric or zwitterionic surfactants may be optionally quaternized secondary or tertiary aliphatic amine derivatives, in which the aliphatic group is a linear or branched chain including from 8 to 22 carbon atoms, said amine derivatives containing at least one anionic group, for instance a carboxylate, sulfonate, sulfate, phosphate or phosphonate group.

Mention may in particular be made of (C₈-C₂₀)alkylbetaines, (C₈-C₂₀)alkylsulfobetaines, (C₈-C₂₀)alkylamido(C₃-C₈)alkylbetaines, (C₈-C₂₀)alkylamido(C₆-C₈)alkylsulfobetaines and (C₈-C₂₀)alkylamido(C₃-C₈)alkylhydroxysultaines.

Among the optionally quaternized derivatives of secondary or tertiary aliphatic amines that may be used, as defined above, mention may also be made of the compounds having the respective formulae (3) and (4) below:

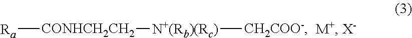

in which:
Rₐ represents a C₁₀ to C₃₀ alkyl or alkenyl group derived from an acid RₐCOOH preferably present in hydrolyzed coconut kernel oil, or a heptyl, nonyl or undecyl group;
R_{b} represents a β-hydroxyethyl group; and
R_{c} represents a carboxymethyl group;
M⁺ represents a cationic counterion derived from an alkali metal or alkaline-earth metal, such as sodium, an ammonium ion or an ion derived from an organic amine, and
X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylarylsulfonates, in particular methyl sulfate and ethyl sulfate; or alternatively M⁺ and X⁻ are absent;

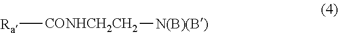

in which:
B represents the group —CH₂CH₂OX′;
B′ represents the group —(CH₂)_{z}Y′, with z=1 or 2;
X′ represents the group —CH₂COOH, —CH₂—COOZ′, —CH₂CH₂COOH or CH₂CH₂—COOZ′, or a hydrogen atom;
Y′ represents the group —COOH, —COOZ′ or —CH₂CH(OH)SO₃H or the group CH₂CH(OH)SO₃—Z′;
Z′ represents a cationic counterion derived from an alkali metal or alkaline-earth metal, such as sodium, an ammonium ion or an ion derived from an organic amine;
R_{a′} represents a C₁₀ to C₃₀ alkyl or alkenyl group of an acid R_{a′}—COOH which is preferably present in hydrolysed linseed oil or coconut kernel oil, an alkyl group, notably a C₁₇ alkyl group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of the compounds of formula (5):

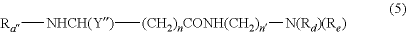

in which:
Y″ represents the group —COOH, —COOZ″ or —CH₂CH(OH)SO₃H or the group CH₂CH(OH)SO₃—Z″;
R_{d} and Rₑ, independently of each other, represent a C₁ to C₄ alkyl or hydroxyalkyl radical;
Z″ represents a cationic counterion derived from an alkali metal or alkaline-earth metal, such as sodium, an ammonium ion or an ion derived from an organic amine;
R_{a″} represents a C₁₀ to C₃₀ alkyl or alkenyl group of an acid R_{a″}—COOH which is preferably present in hydrolysed linseed oil or coconut kernel oil;
n and n′ denote, independently of each other, an integer ranging from 1 to 3.

Among the compounds of formula (5), mention may be made of the compound classified in the CTFA dictionary under the name sodium diethylaminopropyl cocoaspartamide and sold by the company Chimex under the name Chimexane HB.

These compounds may be used alone or as mixtures.

Preferably, the amphoteric or zwitterionic surfactants are chosen, alone or as a mixture, from (C₈-C₂₀)alkylbetaines, (C₈-C₂₀)alkylsulfobetaines, (C₈-C₂₀)alkylamido(C₃-C₈)alkylbetaines, (C₈-C₂₀)alkylamido(C₆-C₈)alkylsulfobetaines and (C₈-C₂₀)alkylamido(C₃-C₈)alkylhydroxysultaines, and also the compounds of formulae (3), (4) and (5) as defined previously.

More preferentially, the amphoteric or zwitterionic surfactants are chosen, alone or as a mixture, from (C₈-C₂₀)alkylbetaines such as cocoylbetaine, (C₈-C₂₀)alkylamido(C₃-C₈)alkylbetaines such as cocamidopropylbetaine, and (C₈-C₂₀)alkylamido(C₃-C₈)alkylhydroxysultaines such as cocamidopropylhydroxysultaine.

Even more preferentially, the amphoteric or zwitterionic surfactants are chosen from (C₈-C₂₀)alkylamido(C₃-C₈)alkylbetaines such as cocamidopropylbetaine, and (C₈-C₂₀)alkylamido(C₃-C₈)alkylhydroxysultaines such as cocamidopropylhydroxysultaine.

Preferably, the amphoteric or zwitterionic compounds are chosen from compounds 1b to 28b below, salts thereof, optical isomers thereof, geometrical isomers thereof, tautomers thereof, solvates thereof and mixtures thereof:

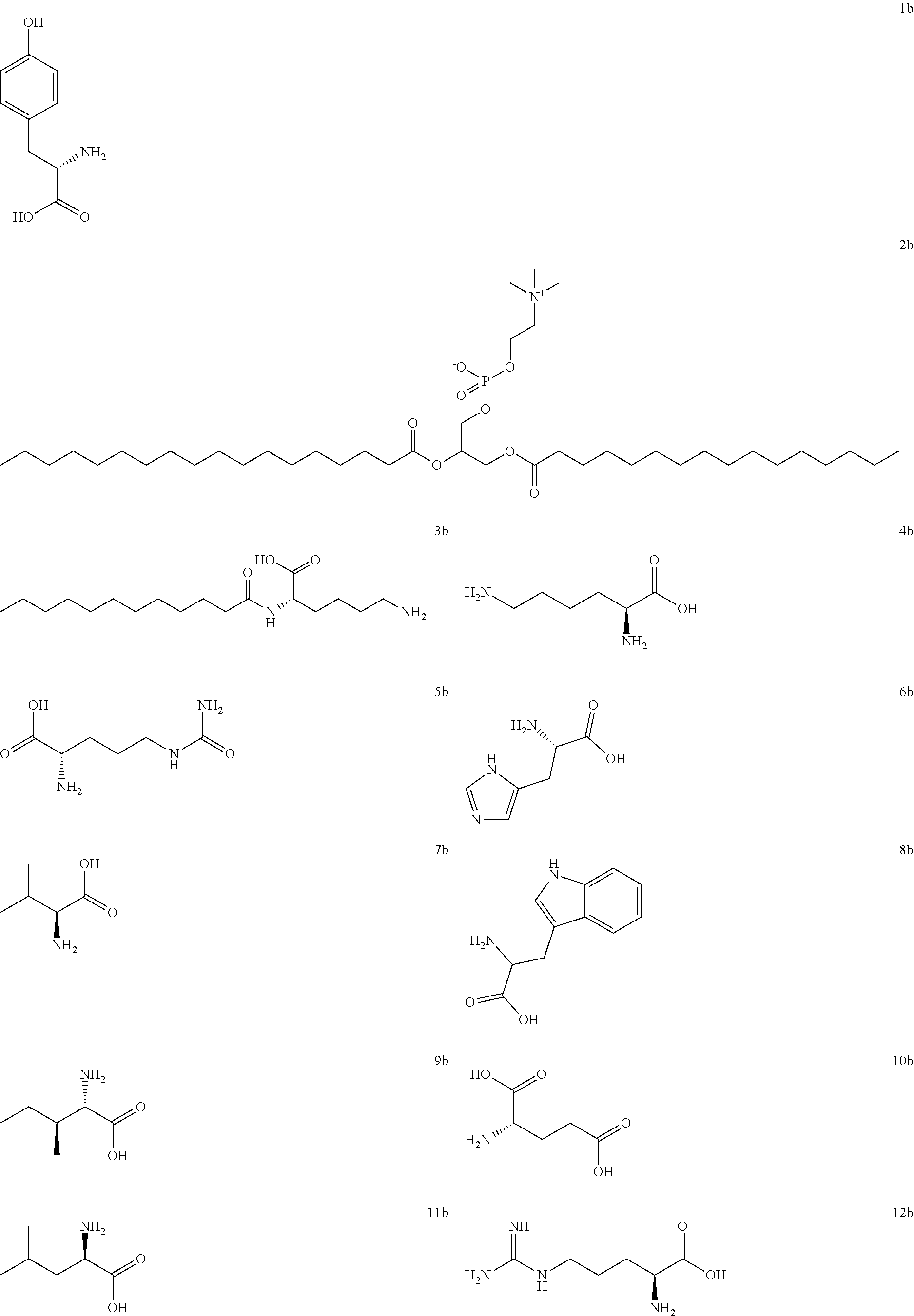

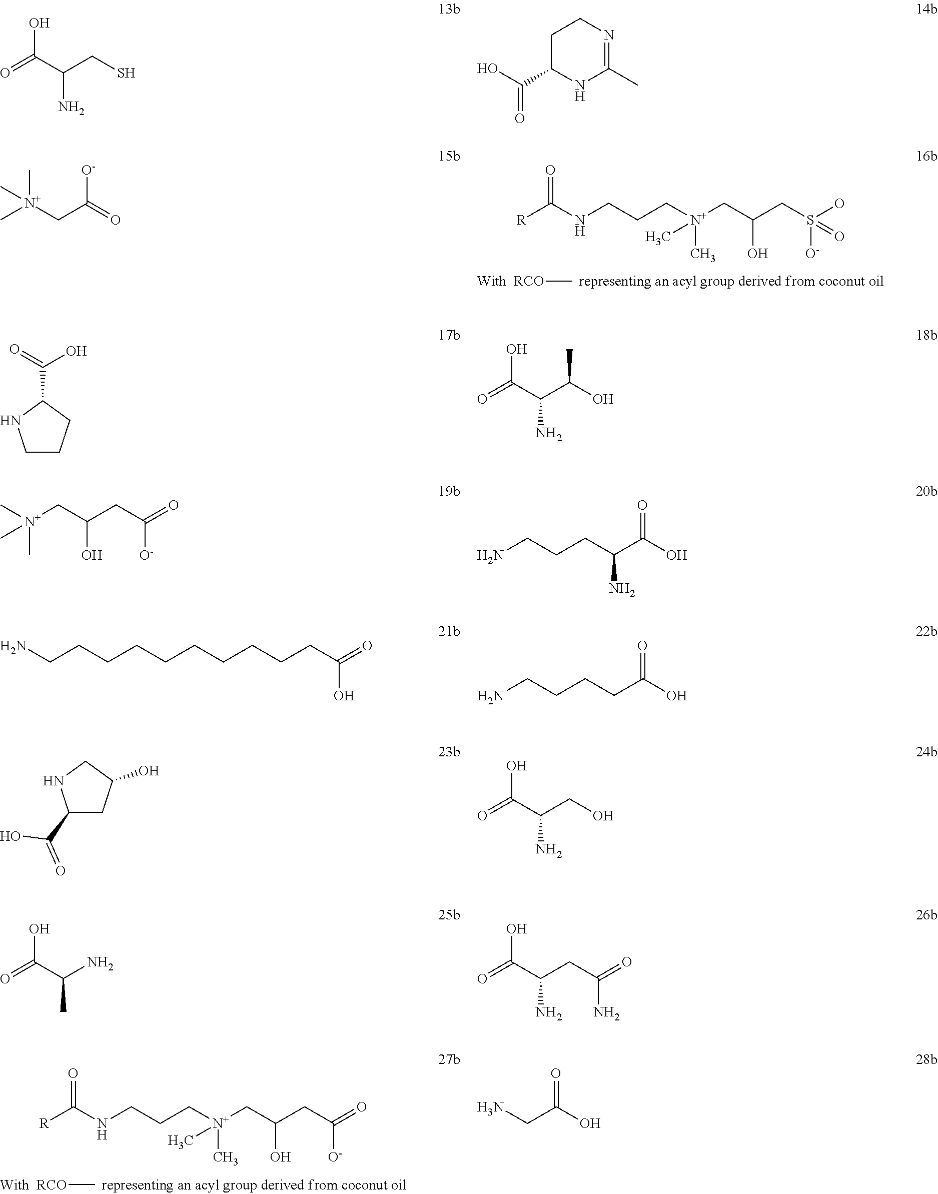

More preferentially, the amphoteric or zwitterionic compounds are chosen from compounds 2b, 4b, 10b, 12b, 15b, 17b, 18b, 24b and 27b, salts thereof, optical isomers thereof, geometrical isomers thereof, tautomers thereof, solvates thereof and mixtures thereof.

Even more preferentially, the amphoteric or zwitterionic compounds are chosen from compounds 4b, 15b, salts thereof, optical isomers thereof, geometrical isomers thereof, tautomers thereof, solvates thereof and mixtures thereof.

Most preferentially, the amphoteric or zwitterionic compounds are chosen from compound 15b, salts thereof, solvates thereof and mixtures thereof.

According to an embodiment, the amphoteric or zwitterionic compounds are of formula R_{b}—CO—X—Z (B1) in which R_{b} represents a (C₁-C₃₀)alkyl group, preferably a (C₁-C₂₀)alkyl group, more preferentially a (C₈-C₁₆)alkyl group, X represents an oxygen atom or NH, preferably NH, and Z represents a linear or branched hydrocarbon-based group containing from 1 to 30 carbon atoms, preferably from 1 to 10 carbon atoms, the group Z being substituted with a carboxylic group (COOH) and an amino group.

As an example of a compound of formula (B1), mention may be made of compound 3b. According to another embodiment, the amphoteric or zwitterionic compounds are of formula R₁—N(R₂)—[Y]ₜ—Z (B2) in which R₁ and R₂ independently represent a hydrogen atom or an alkyl group; preferably, R₁ represents a hydrogen atom and R₂ represents an alkyl group, Y represents CO or CONH, t is 0 or 1, and Z represents a linear or branched hydrocarbon-based group of 1 to 30 carbon atoms, the group Z being substituted with a carboxylic group (COOH) and an amino group.

As examples of compounds of formula (B2), mention may be made of compounds 5b, 20b and 26b.

According to a preferred embodiment, the amphoteric or zwitterionic compounds are of formula R₁—N⁺(R₂)(R₃)—Z′—COO⁻ (B3) in which R₁, R₂ and R₃ independently represent an alkyl group, preferably a C₁-C₂₀ alkyl group such as methyl, said alkyl group being optionally substituted with an amido group —NH—CO—R₄ with R₄ representing a C₁-C₃₀ (hydroxy)alkyl group, Z′ represents a divalent linear or branched hydrocarbon-based group containing from 1 to 30 carbon atoms, the group Z′ being optionally substituted with one or more hydroxyl (OH) groups.

As examples of compounds of formula (B3), mention may be made of compounds 15b, 19b and 27b.

According to another embodiment, the amphoteric or zwitterionic compounds are of formula NH₂—Z′—COOH (B4) in which Z′ represents a linear or branched divalent hydrocarbon-based group of 1 to 30 carbon atoms, preferably of 1 to 10 carbon atoms, more preferentially of 1 to 5 carbon atoms, the group Z′ being optionally substituted with one or more hydroxyl (OH) groups, preferably unsubstituted.

As examples of compounds of formula (B4), mention may be made of compounds 21b, 22b and 28b.

When they are present in the composition, the amphoteric or zwitterionic compounds are preferably present in the composition in a content ranging from 0.01% to 50%, more preferentially ranging from 0.1% to 30%, even more preferentially ranging from 0.2% to 15% by weight relative to the total weight of the composition.

### Esters, Amides and Imines c)

Preferably, the compounds c) are chosen from compounds of formula (Ic) below, salts thereof, optical isomers thereof, geometrical isomers thereof, tautomers thereof, solvates thereof and mixtures thereof:

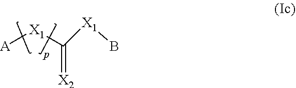

in which formula (Ic):
A represents a linear or branched, saturated or unsaturated hydrocarbon-based group comprising from 1 to 30 carbon atoms, optionally interrupted with one or more non-adjacent oxygen atoms and/or with one or more identical or different groups chosen from: —CO—, —OCO—, —COO—, and/or optionally substituted with one or more identical or different groups chosen from: a hydroxyl group (—OH), a group —COOAk1, a (hetero)cyclic group optionally substituted with one or more hydroxyl groups (—OH);
X₁ independently represents an oxygen atom or a group —NR—, preferably an oxygen atom;
X₂ represents an oxygen atom or a group —NR—, preferably an oxygen atom;
p=0 or 1;
B represents a saturated or unsaturated, linear or branched or cyclic hydrocarbon-based group comprising from 1 to 40 carbon atoms, optionally substituted with one or more identical or different groups chosen from:
a (hetero)cyclic group optionally substituted with one or more identical or different groups chosen from: hydroxyl (—OH), —OR″, —COOA′, —OCOA′, (poly)(hydroxy)alkyl;
a group —OR′;
a group —COOA′;
a group —OCOA′;

and/or optionally interrupted with one or more non-adjacent oxygen atoms and/or with one or more identical or different groups chosen from: —OCO—, —COO—;
A′ represents a linear or branched, saturated or unsaturated hydrocarbon-based group comprising from 1 to 30 carbon atoms, optionally interrupted with one or more non-adjacent oxygen atoms and/or with one or more identical or different groups chosen from: —CO—, —OCO—, —COO—, and/or optionally substituted with one or more identical or different groups chosen from: hydroxyl (—OH), —COOAk1;
R represents a hydrogen atom or an alkyl group, in particular a (C₁-C₄)alkyl group;
R′ represents a hydrogen atom or an alkyl group optionally substituted with one or more identical or different groups chosen from: hydroxyl (—OH) or —OCOA′;
R″ represents:
an alkyl group optionally substituted with one or more hydroxyl groups (—OH); or
a (hetero)cyclic group optionally substituted with one or more identical or different groups chosen from: hydroxyl (—OH), (poly)(hydroxy)alkyl;
Ak1 represents an alkyl group optionally substituted with —COOAk2;
Ak2 represents an alkyl group;

it being possible for R and B together with the nitrogen atom bearing them to form a heterocycle;

it being possible for A and B together to form a heterocycle optionally substituted notably with one or more identical or different groups chosen from:
a (hetero)cyclic group optionally substituted notably with one or more identical or different groups chosen from: hydroxyl (—OH), (poly)(hydroxy)alkyl;
a hydroxyl group (—OH);
a group —OR″;
a (poly)(hydroxy)alkyl group;

it being understood that if X₁ represents a group —NR—, then A and B may represent a hydrogen atom.

More preferentially, the compounds c) are chosen from the following compounds 1c to 94c, salts thereof, optical isomers thereof, geometrical isomers thereof, tautomers thereof, solvates thereof and mixtures thereof:

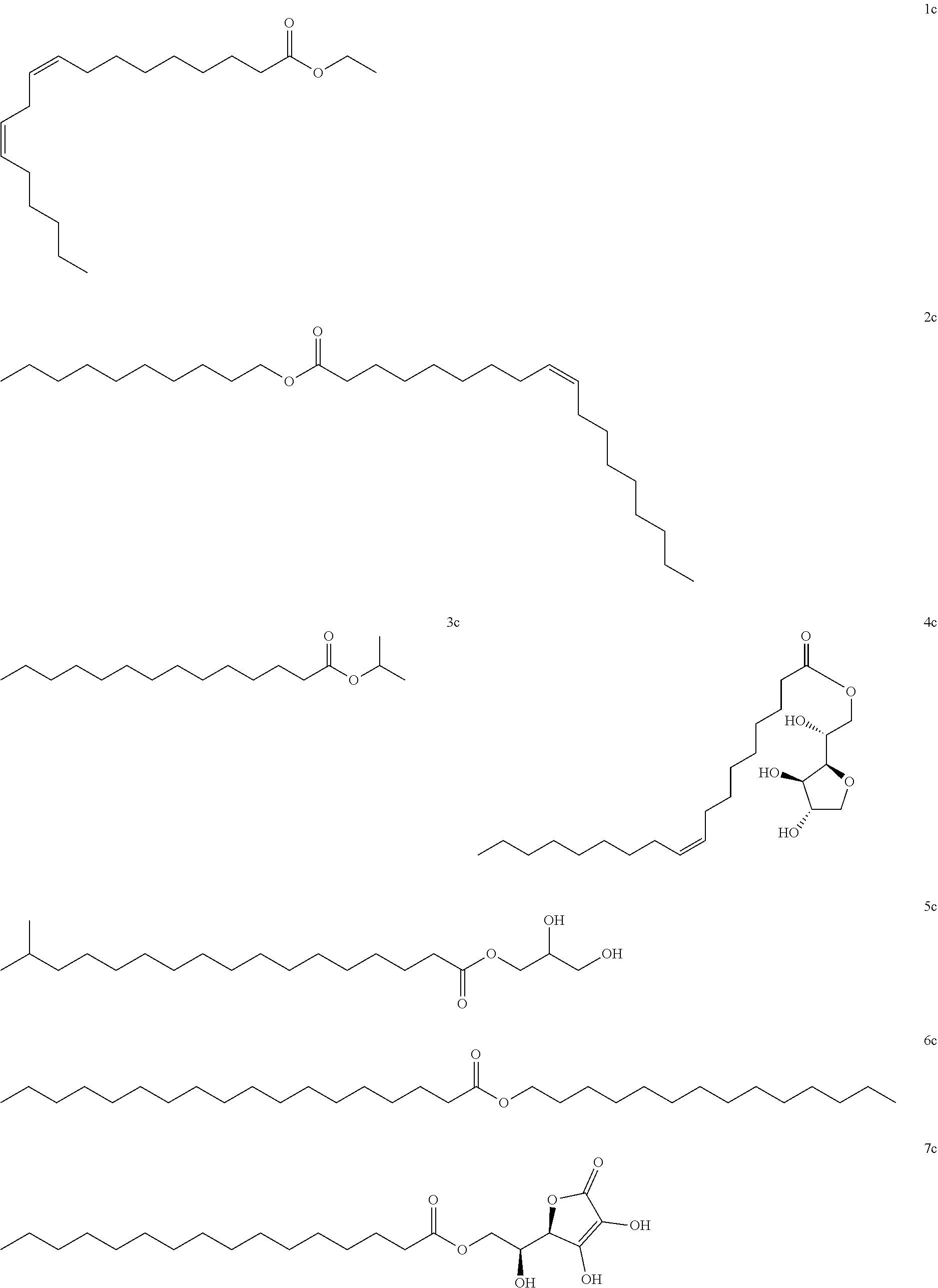

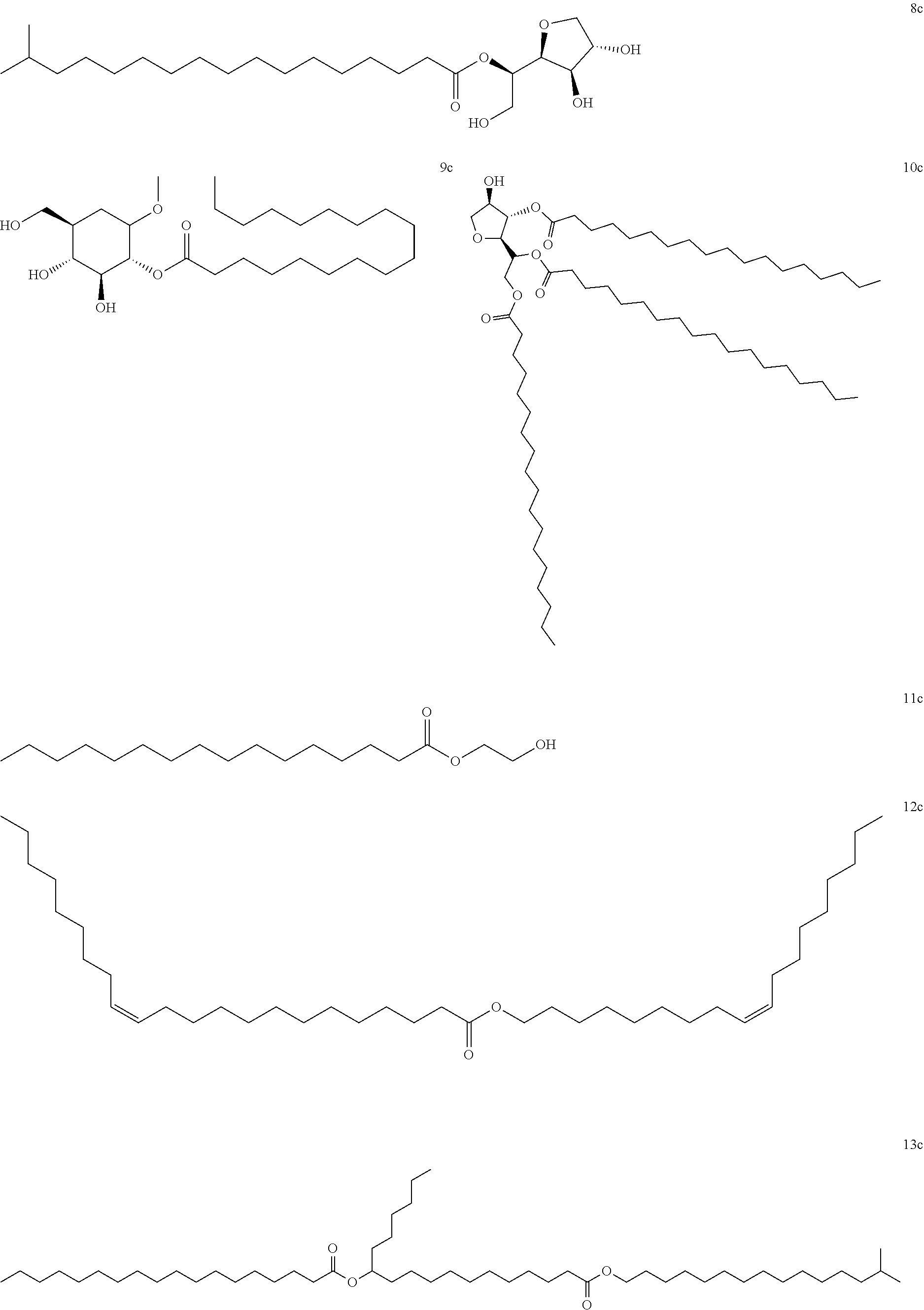

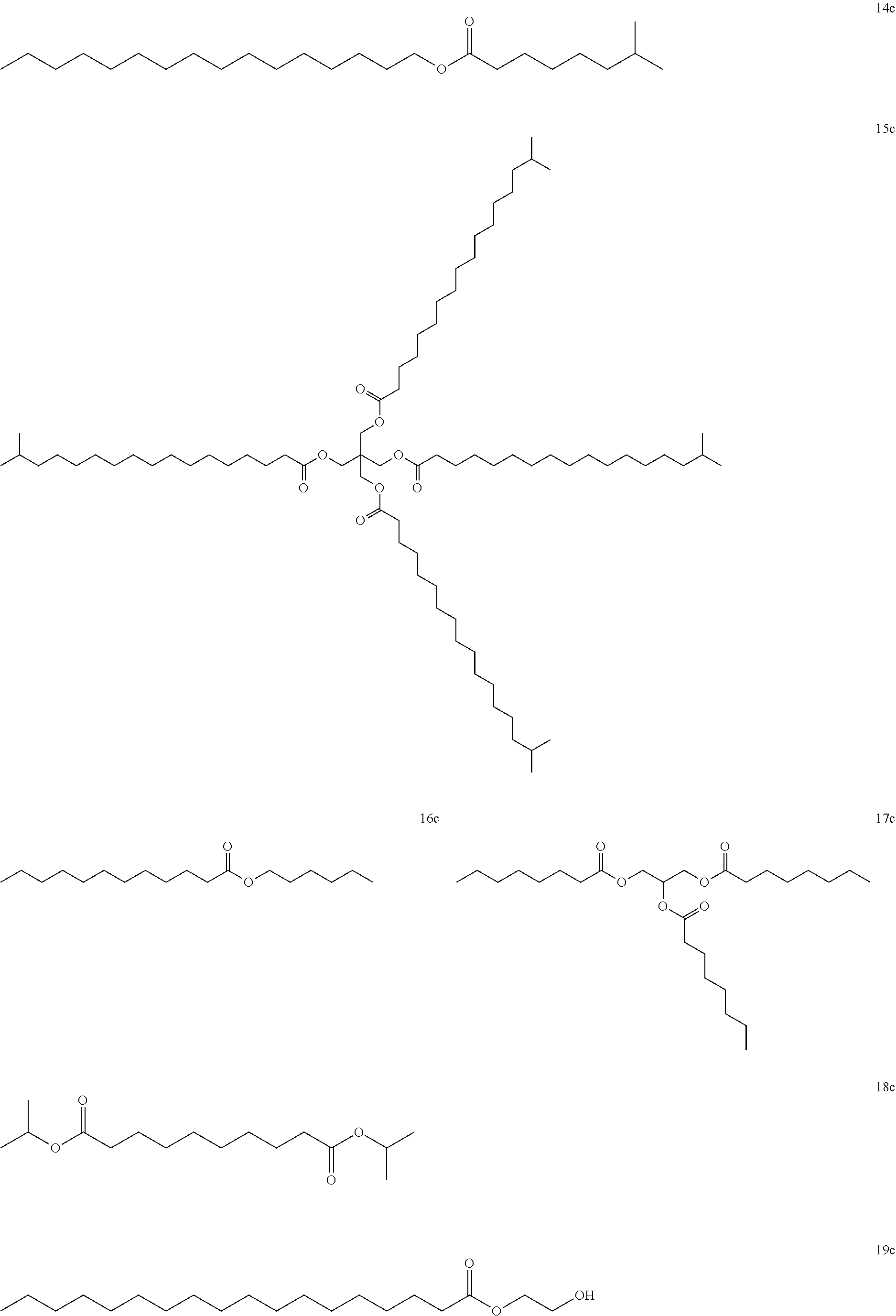

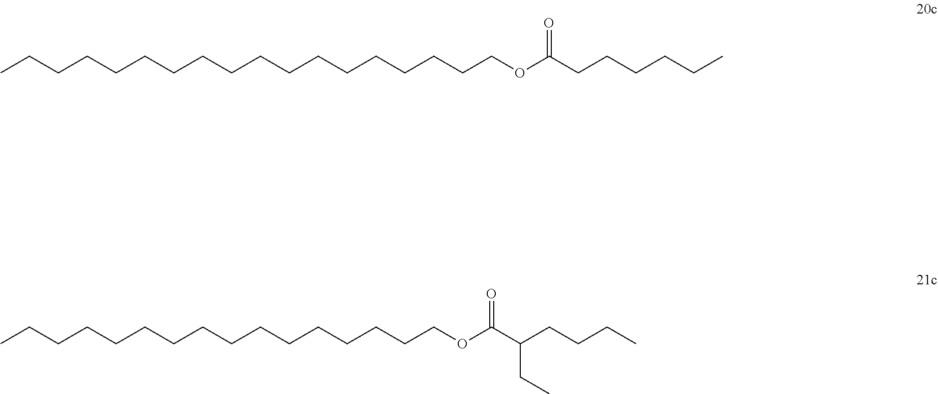

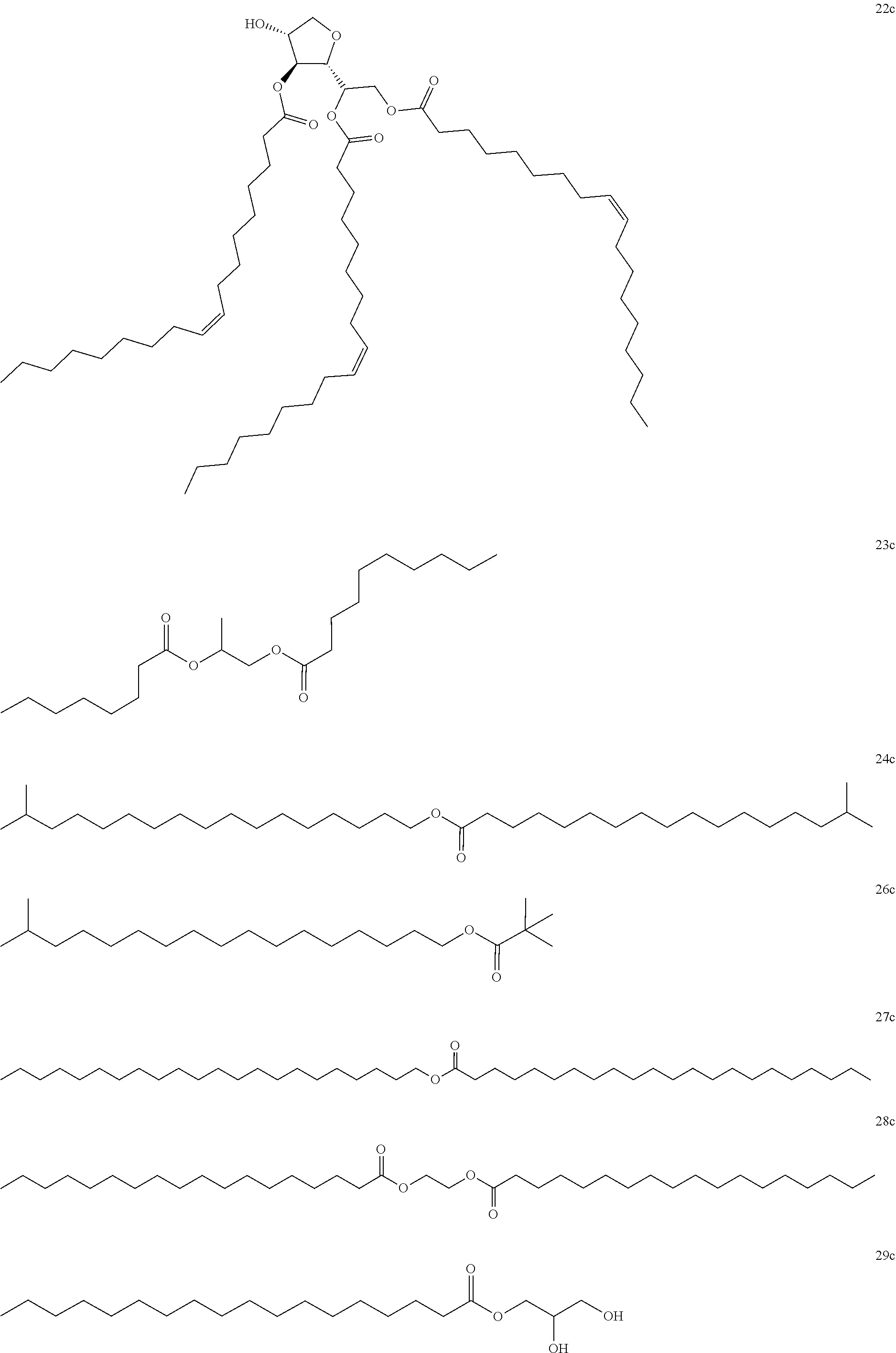

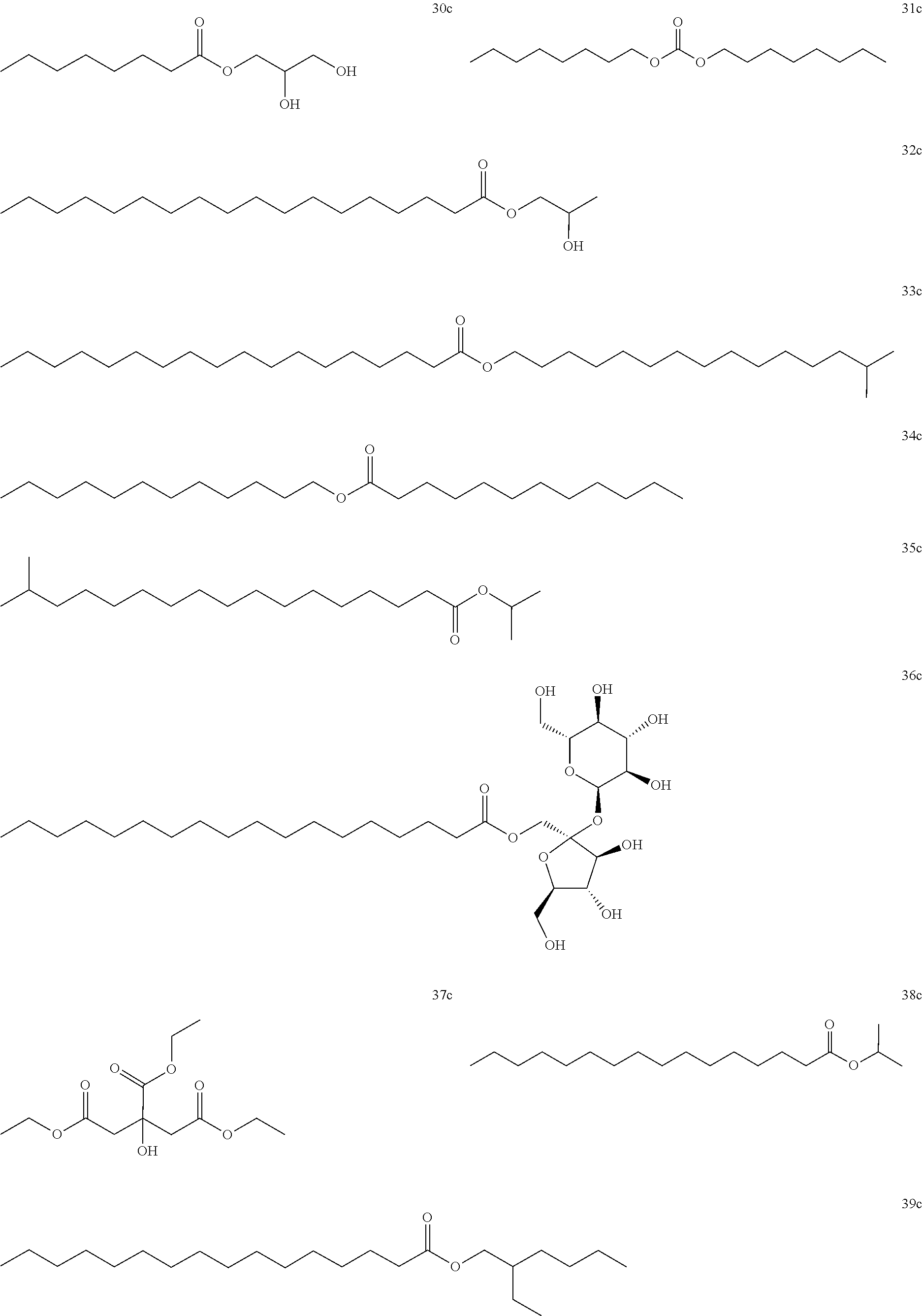

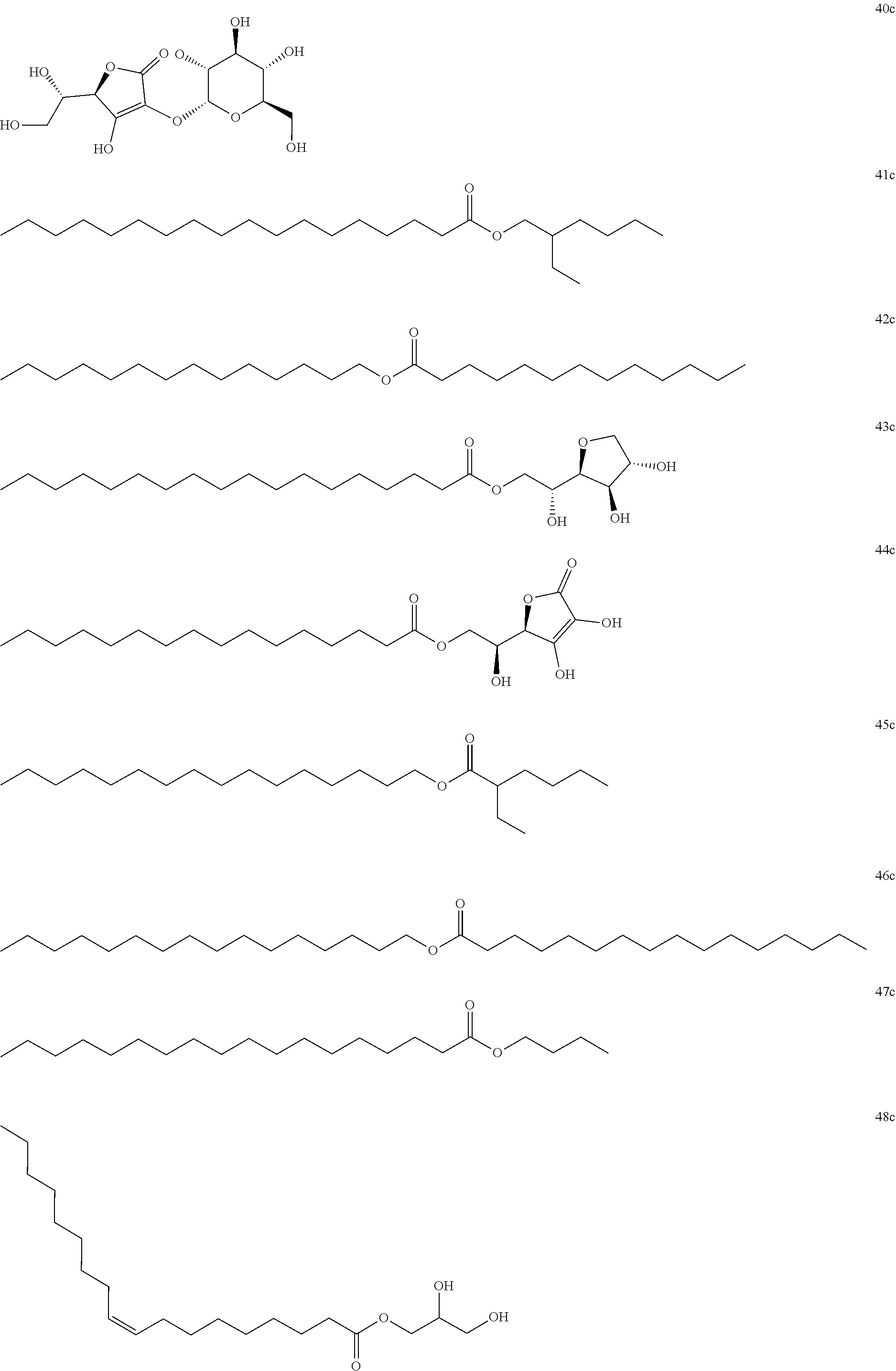

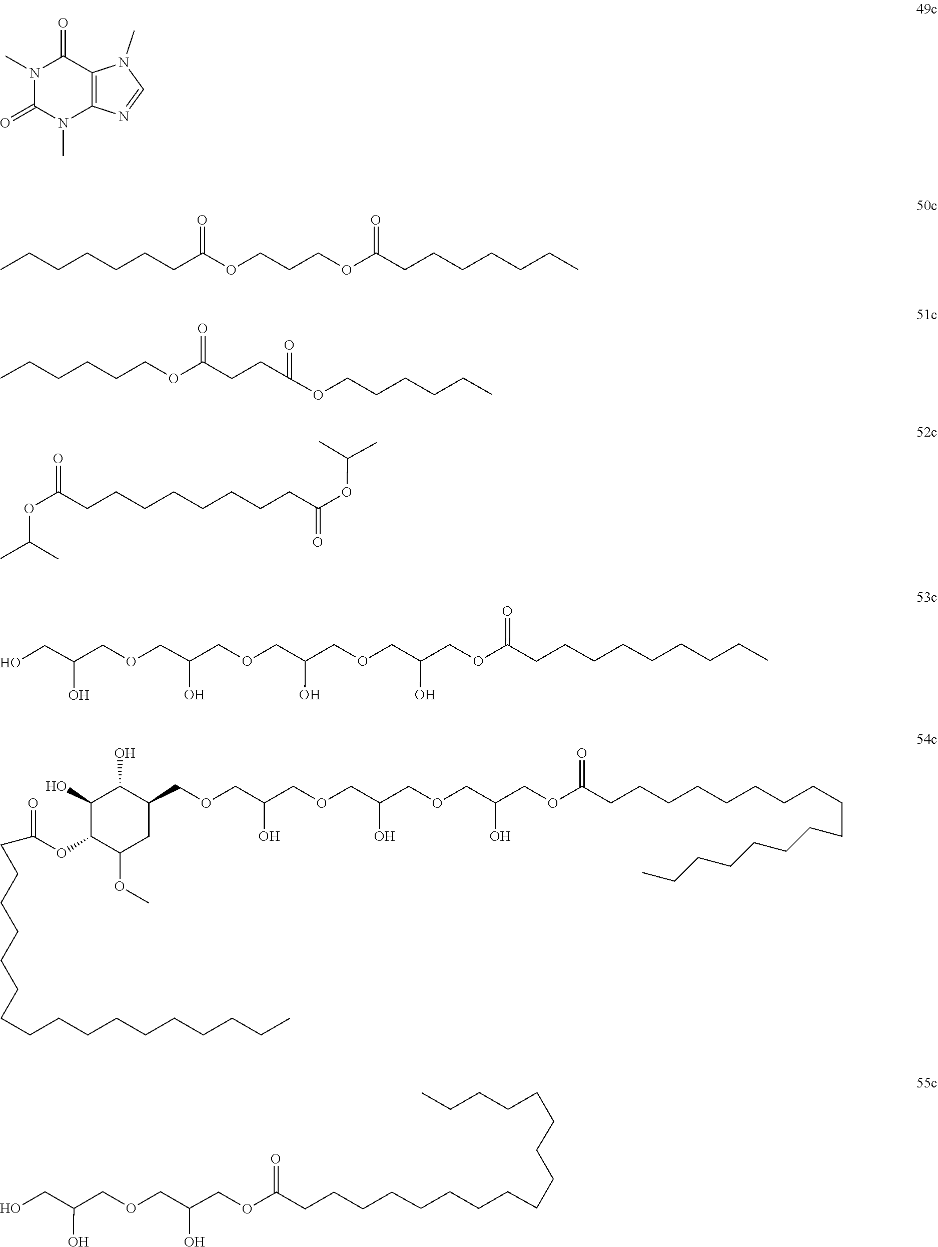

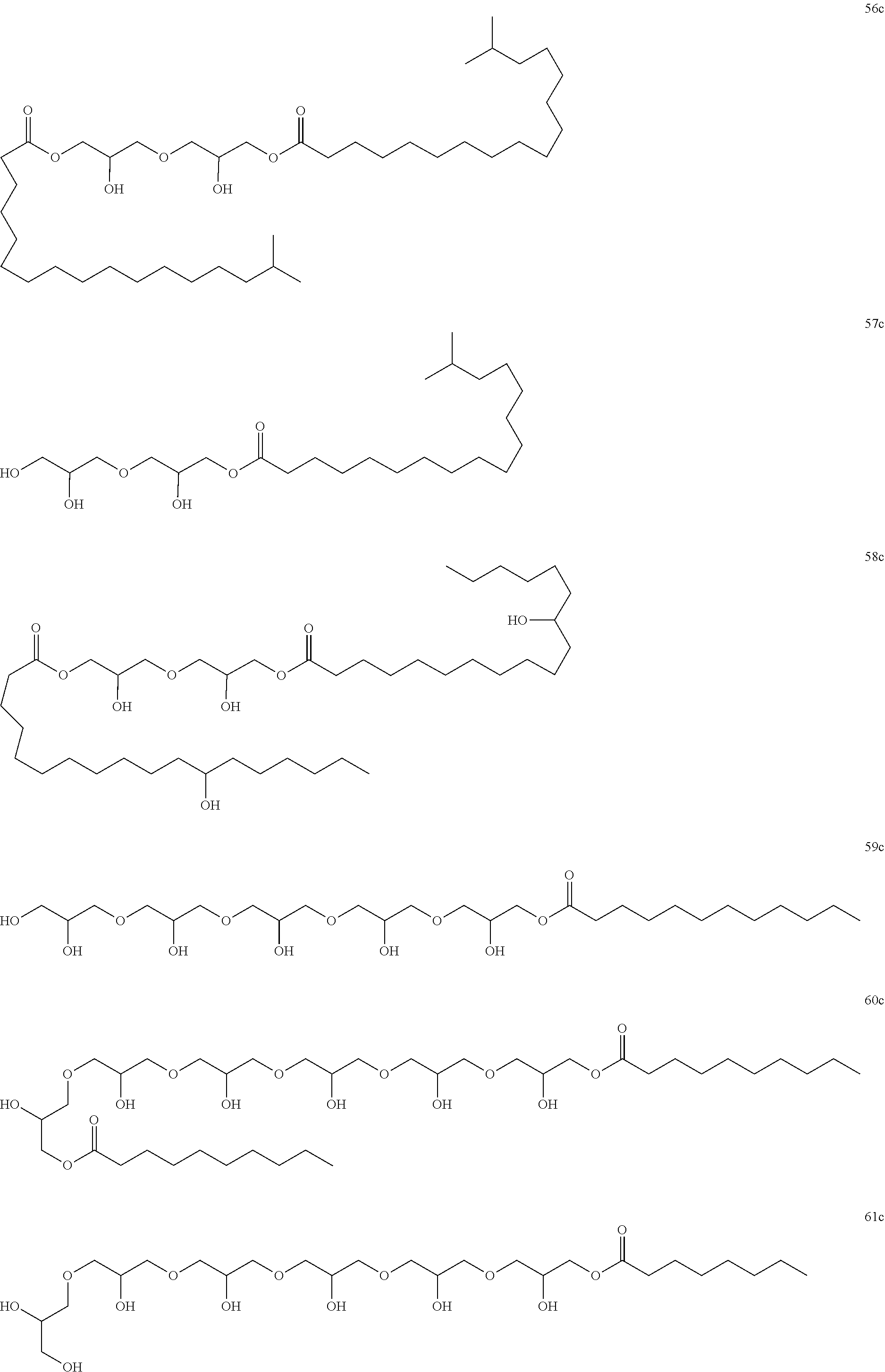 [SMILES: CC(C)CCCCCCCCCCCCCCC(=O)OCC(O)COCC(O)COC(=O)CCCCCCCCCCCCCCC(C)C ; CC(C)CCCCCCCCCCCCCCC(=O)OCC(O)COCC(O)CO ; CCCCCCC(O)CCCCCCCCCCC(=O)OCC(O)COCC(O)COC(=O)CCCCCCCCCCC(O)CCCCCC ; CCCCCCCCCCCC(=O)OCC(O)COCC(O)COCC(O)COCC(O)COCC(O)CO ; CCCCCCCCCC(=O)OCC(O)COCC(O)COCC(O)COCC(O)COCC(O)COCC(O)COC(=O)CCCCCCCCC ; CCCCCCCC(=O)OCC(O)COCC(O)COCC(O)COCC(O)COCC(O)COCC(O)CO]

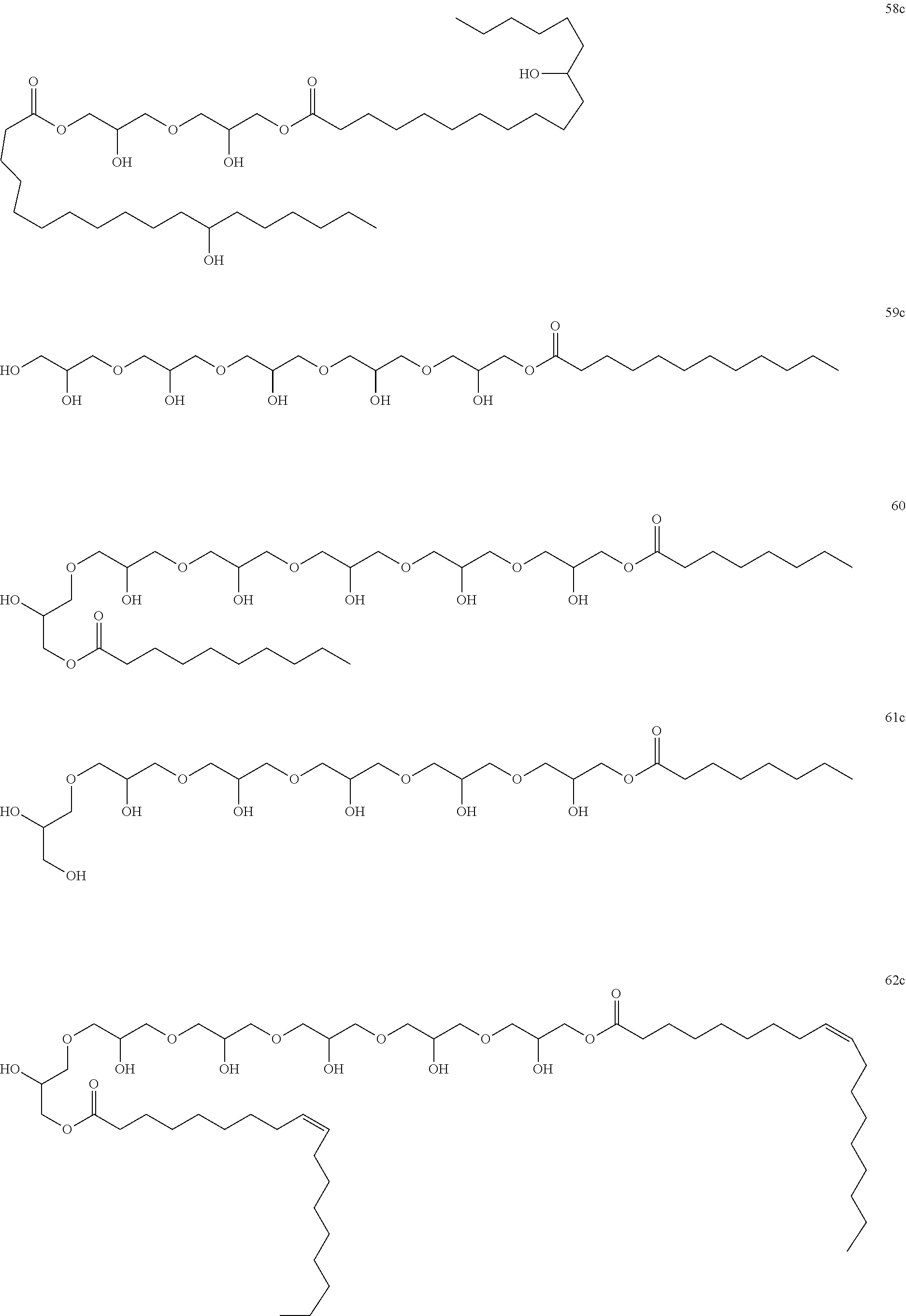 [SMILES: CCCCCCC(O)CCCCCCCCCCC(=O)OCC(O)COCC(O)COC(=O)CCCCCCCCCCC(O)CCCCCC ; CCCCCCCCCCCC(=O)OCC(O)COCC(O)COCC(O)COCC(O)COCC(O)CO ; CCCCCCCCCC(=O)OCC(O)COCC(O)COCC(O)COCC(O)COCC(O)COCC(O)COC(=O)CCCCCCC ; CCCCCCCC(=O)OCC(O)COCC(O)COCC(O)COCC(O)COCC(O)COCC(O)CO ; CCCCCCCC/C=C\CCCCCCCC(=O)OCC(O)COCC(O)COCC(O)COCC(O)COCC(O)COCC(O)COC(=O)CCCCCCC/C=C\CCCCCCCC]

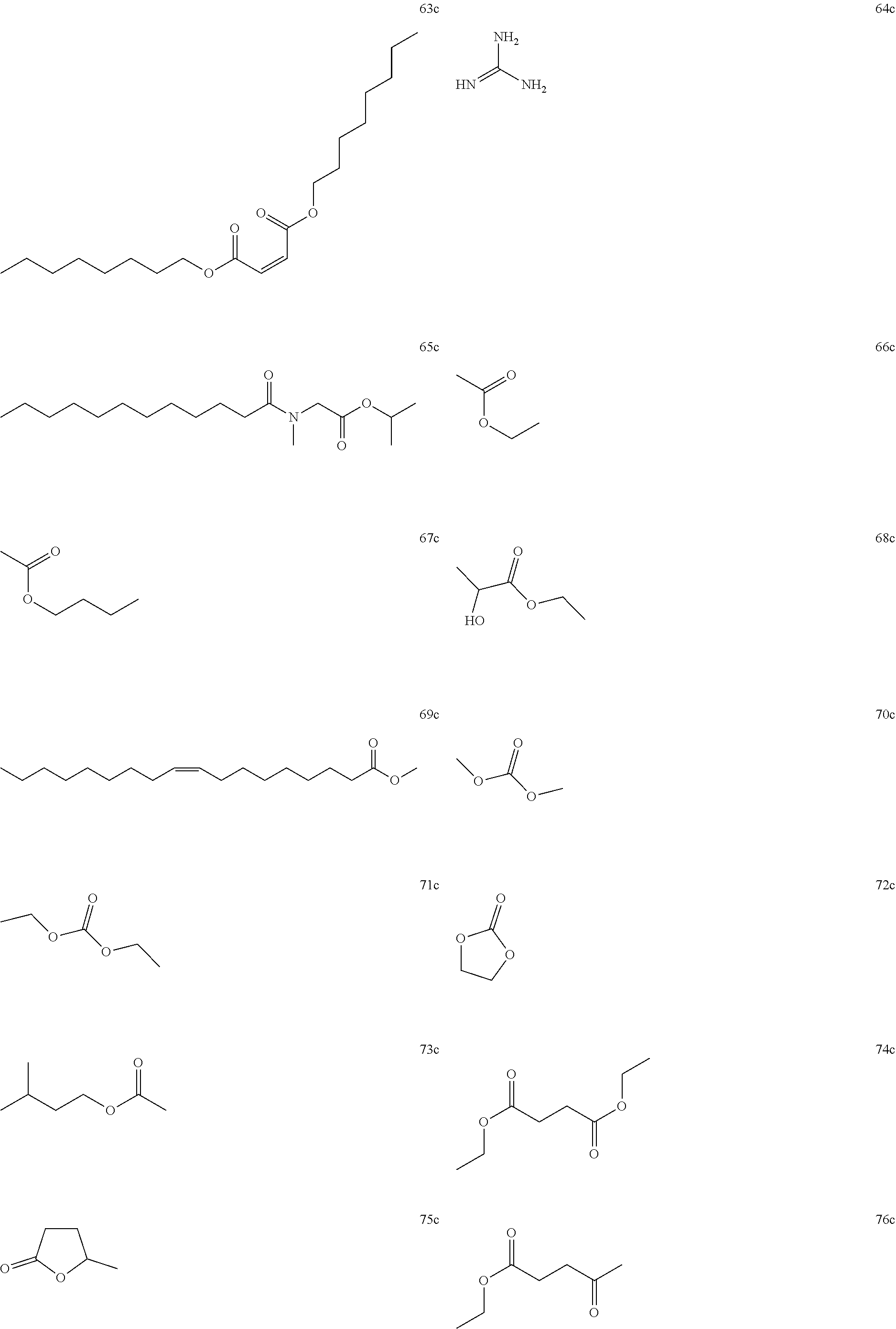

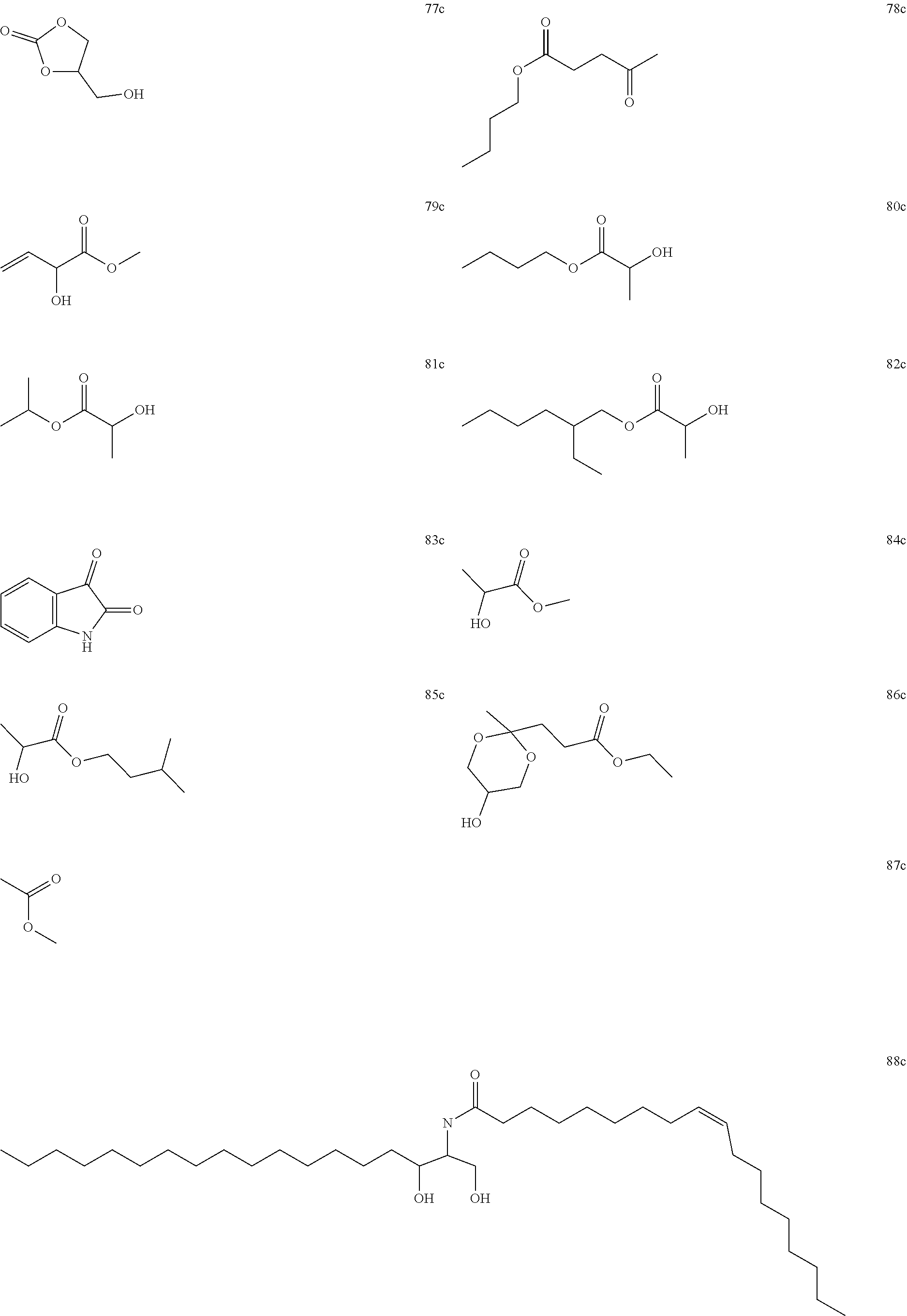

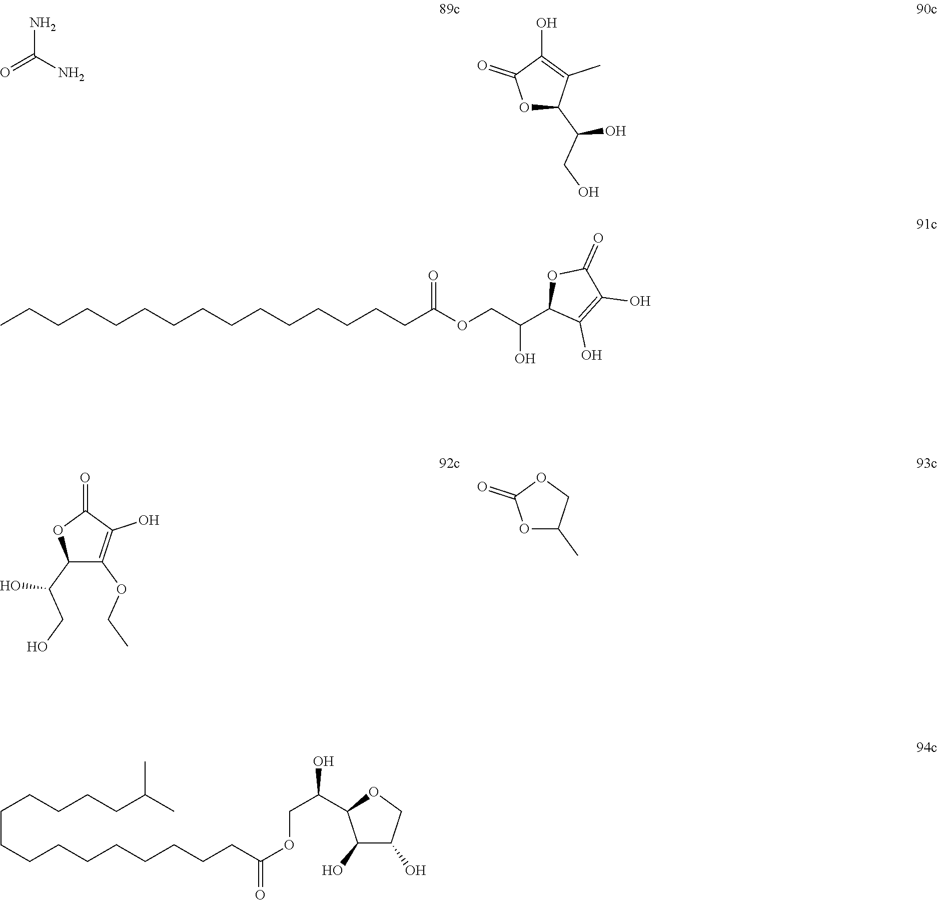

Even more preferentially, the compounds c) are chosen from compounds 1c to 63c and 94c, salts thereof, optical isomers thereof, geometrical isomers thereof, tautomers thereof, solvates thereof and mixtures thereof.

Preferably, the compounds c) are chosen from compounds 1c, 4c, 5c, 6c, 8c, 12c, 15c, 16c, 22c, 24c, 26c, 27c, 31c, 33c, 37c, 41c, 53c, 54c, 55c, 58c, 60c, 62c, 63c and 94c, salts thereof, optical isomers thereof, geometrical isomers thereof, tautomers thereof, solvates thereof and mixtures thereof.

Even more preferably, the compounds c) are chosen from compounds 4c, 58c, salts thereof, optical isomers thereof, geometrical isomers thereof, tautomers thereof, solvates thereof and mixtures thereof.

According to a preferred variant of the invention, the compounds c) are chosen from the compounds of formula (Ic), salts thereof, optical isomers thereof, geometrical isomers thereof, tautomers thereof, solvates thereof and mixtures thereof, in which formula (Ic) p=0 and X1 and X2 represent an oxygen atom. According to this variant, the compounds c) preferably denote the compounds of formula (C1) hereinbelow, salts thereof, optical isomers thereof, geometrical isomers thereof, tautomers thereof, solvates thereof and mixtures thereof:

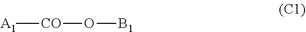

in which:
A₁ represents a C₁-C₄₀ alkyl or alkenyl group, optionally substituted with one or more hydroxyl (OH) groups, and/or optionally interrupted with —OCO—, CO—O—, —O— or CO—NR or CO with R as defined previously, preferably a (C₁-C₄)alkyl group such as methyl;
B₁ represents a C₁-C₄₀ alkyl or alkenyl group, optionally substituted with one or more identical or different groups chosen from: a hydroxyl group (OH), a (hetero)cyclic group, said (hetero)cyclic group being optionally substituted with one or more groups chosen from hydroxyl (OH), —OR_{b}, —O—CO-A₃ with A₃ representing a C₁-C₄₀ alkyl or alkenyl group and R_{b} representing a preferably C₁-C₆ alkyl group; and/or optionally interrupted with —O—CO—, —CO—O—, —CO—NR′ₐ—, —NR′ₐ—CO—, an oxygen atom —O— or —CO— with R′ₐ representing a hydrogen atom or a C₁-C₁₀ alkyl group, preferably C₁-C₄ alkyl, such as methyl.

As examples of compounds of formula (C1), mention may be made of compounds 1c to 8c, 9c to 30c, 32c to 35c, 36c to 39c, 41c to 48c, 50c to 63c, 65c to 69c, 73c, 74c, 76c, 78c à 82c, 84c, 85c, 91c, salts thereof, optical isomers thereof, geometrical isomers thereof, tautomers thereof, solvates thereof and mixtures thereof, in particular compounds 1c, 4c, 5c, 6c, 8c, 12c, 15c, 16c, 22c, 24c, 26c, 27c, 33c, 37c, 41c, 53c, 54c, 55c, 58c, 60c, 62c and 63c, salts thereof, optical isomers thereof, geometrical isomers thereof, tautomers thereof, solvates thereof and mixtures thereof, in particular compounds 8c and 58c, salts thereof, optical isomers thereof, geometrical isomers thereof, tautomers thereof, solvates thereof and mixtures thereof.

According to another variant of the invention, the compounds c) are chosen from the compounds of formula (Ic), salts thereof, optical isomers thereof, geometrical isomers thereof, tautomers thereof, solvates thereof and mixtures thereof, in which formula (Ic) p=0 and X1 and X2 denote an oxygen atom, and the compounds of formula (C2) hereinbelow, salts thereof, optical isomers thereof, geometrical isomers thereof, tautomers thereof, solvates thereof and mixtures thereof:

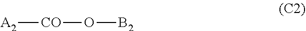

in which:
A₂ and B₂ together with the atoms bearing them form an aromatic or non-aromatic heterocyclic group, optionally substituted notably with one or more identical or different groups chosen from:
a (hetero)cyclic group optionally substituted notably with one or more identical or different groups chosen from: hydroxyl (—OH), (poly)(hydroxy)alkyl;
a hydroxyl group (—OH);
a group —OR″ with R″ as defined previously; in particular, R″ denotes a C₁-C₆ alkyl radical such as ethyl;
a (poly)(hydroxy)alkyl group.

As examples of compounds of formula (C2), mention may be made of compounds 40c, 75c, 90c, 92c, salts thereof, optical isomers thereof, geometrical isomers thereof, tautomers thereof, solvates thereof and mixtures thereof.

According to another variant of the invention, the compounds c) are chosen from the compounds of formula (Ic), salts thereof, optical isomers thereof, geometrical isomers thereof, tautomers thereof, solvates thereof and mixtures thereof, in which formula (Ic) p=1 and X1 and X2 denote an oxygen atom.

According to this variant, the compounds c) preferably denote the compounds of formula (C3) hereinbelow, salts thereof, optical isomers thereof, geometrical isomers thereof, tautomers thereof, solvates thereof and mixtures thereof:

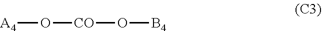

in which:
A₄ and B₄ independently represent a C₁-C₄₀, preferably C₁-C₂₀, more preferentially C₁-C₁₀, alkyl or alkenyl group, optionally substituted with one or more hydroxyl (OH) groups, —OR″ with R″ as defined previously, in particular R″ represents a C₁-C₆ alkyl group such as methyl or (poly)(hydroxy)alkyl, which is preferably unsubstituted, and/or optionally interrupted with —OCO—, CO—O—, —O—, CO—NRₐ with Rₐ denoting a hydrogen atom or a preferably C₁-C₄ alkyl or CO group, preferably interrupted with —O—, more preferentially uninterrupted; or

A₄ and B₄ together with the atoms bearing them form an aromatic or non-aromatic heterocyclic group optionally substituted notably with one or more identical or different groups chosen from:
a (hetero)cyclic group optionally substituted notably with one or more identical or different groups chosen from: hydroxyl (—OH), (poly)(hydroxy)alkyl;
a hydroxyl group (—OH);
a group —OR″ with R″ as defined previously; in particular, R″ represents a C₁-C₆ alkyl radical such as methyl;
a (poly)(hydroxy)alkyl group such as a C₁-C₆ alkyl group, a C₁-C₆ hydroxyalkyl group or a C₁-C₆ polyhydroxyalkyl group.

Preferably, the compounds of formula (C3) are such that A₄ and B₄ are identical.

Preferably, A₄ and B₄ independently denote a preferably linear C₁-C₁₀ alkyl radical such as methyl, ethyl, propyl, isopropyl, butyl, isobutyl, tert-butyl, heptyl or octyl.

Preferably, when A₄ and B₄ together with the atoms which bear them form an aromatic or non-aromatic heterocyclic group, said heterocyclic group is optionally substituted with one or more identical or different groups chosen from:
a group —OR″ with R″ as defined previously; in particular, R″ represents a C₁-C₆ alkyl radical such as methyl or ethyl;
a (poly)(hydroxy)alkyl group such as a C₁-C₆ alkyl group, a C₁-C₆ hydroxyalkyl group or a C₁-C₆ polyhydroxyalkyl group.

As examples of compounds of formula (C3), mention may be made of the compounds 31c, 70c, 71c, 72c, 77c, 93c, salts thereof, optical isomers thereof, geometrical isomers thereof, tautomers thereof, solvates thereof and mixtures thereof.

According to another variant of the invention, the compounds c) are chosen from the compounds of formula (Ic), salts thereof, optical isomers thereof, geometrical isomers thereof, tautomers thereof, solvates thereof and mixtures thereof, in which formula (Ic) p=0 and X1 and X2 denote an oxygen atom.

According to this variant, the compounds c) preferably denote the compounds of formula (C4) hereinbelow, salts thereof, optical isomers thereof, geometrical isomers thereof, tautomers thereof, solvates thereof and mixtures thereof:

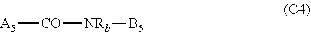

in which:
A₅ and B₅ independently represent a C₁-C₄₀, preferably C₁-C₂₀, alkyl or alkenyl group, optionally substituted with one or more hydroxyl groups (OH), —OR″ with R″ as defined previously; in particular, R″ represents a C₁-C₆ alkyl group such as methyl, or (poly)(hydroxy)alkyl, which is preferably unsubstituted, and/or optionally interrupted with —OCO—, CO—O—, —O—, CO—NRₐ with Rₐ representing a hydrogen atom or a preferably C₁-C₄, alkyl group, or CO, preferably interrupted with —O— more preferentially uninterrupted; or

A₅ and B₅ together with the atoms bearing them form an aromatic or non-aromatic heterocyclic group optionally substituted notably with one or more identical or different groups chosen from:
a heterocyclic group optionally substituted notably with one or more identical or different groups chosen from: hydroxyl (—OH) and (poly)(hydroxy)alkyl, said heterocyclic group also possibly containing one or more CO;
a hydroxyl group (—OH);
a group —OR″ with R″ as defined previously; in particular, R″ represents a C₁-C₆ alkyl group such as methyl or ethyl;
a (poly)(hydroxy)alkyl group such as a C₁-C₆ alkyl group, a C₁-C₆ hydroxyalkyl group or a C₁-C₆ polyhydroxyalkyl group; and
R_{b} denotes a hydrogen atom or a C₁-C₂₀ alkyl radical, preferably C₁-C₁₀ alkyl, more preferentially C₁-C₄ alkyl, such as methyl or ethyl.

As examples of compounds of formula (C4), mention may be made of compounds 88c, 71c, 72c, 77c, 93c, salts thereof, optical isomers thereof, geometrical isomers thereof, tautomers thereof, solvates thereof and mixtures thereof.

According to another variant of the invention, the compounds c) are chosen from the compounds of formula (Ic), salts thereof, optical isomers thereof, geometrical isomers thereof, tautomers thereof, solvates thereof and mixtures thereof, in which formula (Ic) p=1 and X1 represents a group —NR— and X2 represents a group —NR— or an oxygen atom, with R representing a hydrogen atom or an alkyl group, in particular a (C₁-C₄)alkyl group;

According to this variant, the compounds c) preferably denote the compounds of formula (C5) hereinbelow, salts thereof, optical isomers thereof, geometrical isomers thereof, tautomers thereof, solvates thereof and mixtures thereof:

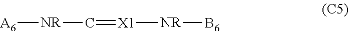

in which:
A₆ and B₆ independently represent a hydrogen atom, a C₁-C₂₀ preferably C₁-C₁₀ alkyl or alkenyl group, optionally substituted with one or more hydroxyl (OH) groups, —OR″ or (poly)(hydroxy)alkyl, which is preferably unsubstituted, and/or optionally interrupted with —OCO—, CO—O—, —O—, or CO, which is preferably uninterrupted; or

A₆ and B₆ together with the atoms bearing them form an aromatic or non-aromatic heterocyclic group optionally substituted notably with one or more identical or different groups chosen from:
a hydroxyl group (—OH);
a group —OR″ with R″ as defined previously; in particular, R″ denotes a C₁-C₆ alkyl radical such as methyl;
a (poly)(hydroxy)alkyl group such as a C₁-C₆ alkyl group, a C₁-C₆ hydroxyalkyl group or a C₁-C₆ polyhydroxyalkyl group, preferably a C₁-C₄ alkyl group such as methyl;
R represents a hydrogen atom or an alkyl group, in particular a (C₁-C₄)alkyl group such as methyl or ethyl.

Preferably, when A₆ and B₆ together with the atoms bearing them form an aromatic or non-aromatic heterocyclic group, said heterocyclic group is optionally substituted with one or more identical or different groups chosen from C₁-C₆ alkyl groups, preferably C₁-C₄ alkyl groups such as methyl.

As examples of compounds of formula (C5), mention may be made of compounds 49c, 64c, salts thereof, optical isomers thereof, geometrical isomers thereof, tautomers thereof, solvates thereof and mixtures thereof.

When they are present in the composition, the compounds c) are preferably present in the composition in a content ranging from 0.01% to 50%, more preferentially ranging from 0.1% to 30%, even more preferentially ranging from 0.2% to 15% by weight relative to the total weight of the composition.

### Organic Amines d)

The organic amines of the invention may or may not be polymeric.

According to a preferred embodiment of the invention, the polymeric organic amines are polyaminated and hydrocarbon-based and thus do not comprise any Si atoms.

The organic amines may notably be chosen from amino polymers, with a weight-average molecular weight ranging from 500 g·mol⁻¹ to 1 000 000 g·mol⁻¹, preferably ranging from 500 g·mol⁻¹ to 500 000 g·mol⁻¹, and preferentially ranging from 500 g·mol⁻¹ to 100 000 g·mol⁻¹.

According to a particular embodiment of the invention, the organic amines are diamines and are chosen in particular from polyether diamines notably of formula H₂N-ALK-O-[ALK′-O]ₘ-ALK″-NH₂ with ALK, ALK′ and ALK″, which may be identical or different, representing a linear or branched (C₁-C₆))alkylene group, and m representing an integer greater than or equal to 0, such as 4,7,10-trioxa-1,13-tridecanediamine or the compounds known under the reference Jeffamine from the company Hunstman, and more particularly α,ω-diamino polyethylene glycol and/or polypropylene glycol (with an amine function at the end of the chain) such as the products sold under the names Jeffamine D-230, D-400, D-2000, D-4000, ED-600, ED-9000 and ED-2003.

According to a particular embodiment of the invention, the organic amines are triaminated, i.e. they contain three primary and/or secondary amine groups, preferably primary amine groups (NH₂). More particularly, they are chosen from polyether triamines notably of formula ALK′″[(O-ALK′)ₘ-NH₂]₃ with ALK′ as defined previously and ALK′″ representing a linear or branched trivalent (C₁-C₆)alkylene group, and m representing an integer greater than or equal to 0.

As triamino organic amines, polyether triamines are particularly mentioned, and notably α,ω-diamino polyethylene glycol and/or polypropylene glycol (with an amine function at the end of the chain) such as the products sold under the names Jeffamine T-403. According to another particular embodiment of the invention, the organic amines include more than three primary and/or secondary amine groups, preferably primary amine groups (NH₂).

In this variant, the organic amines are chosen from poly(meth)acrylates or poly(meth)acrylamides bearing lateral primary or secondary amine functions, such as poly(3-aminopropyl)methacrylamide and poly(2-aminoethyl) methacrylate.

According to another embodiment, the organic amines are chosen from chitosans (notably poly(D-glucosamine)) and polydimethylsiloxanes comprising primary amine groups at the end of the chain and/or on side chains.

According to another embodiment, the organic amines are chosen in particular from poly(alkylene (C₂-C₅)imines), and preferably polyethylenimines and polypropyleneimines, notably poly(ethyleneimine), in particular the product sold under reference 408700 by the company Aldrich Chemical or under the trade name Lupasol by BASF, notably with a molecular weight of between 1200 and 25 000; poly(allylamine), in particular the product sold under reference 479136 by the company Aldrich Chemical; polyvinylamines and copolymers thereof, notably with vinylamides, in particular vinylamine/vinylformamide copolymers such as those sold under the name Lupamin® 9030 by the company BASF; polyamino acids containing NH₂ groups, such as polylysine, notably the product sold by the company JNC Corporation (formerly Chisso); amino dextran, in particular the product sold by CarboMer Inc; amino polyvinyl alcohol, in particular the product sold by CarboMer Inc; acrylamido(C₁-C₆)alkylamine-based copolymers, notably acrylamidopropylamine-based copolymers; and poly(D-Glucosamine), for example sold under the reference Kionutrime CSG® by the company Kytozyme.

As organic amines, mention may also be made of α,ω-diamino polytetrahydrofurans (or polytetramethylene glycol) and α,ω-diamino polybutadienes.

According to a particular embodiment of the invention, the organic amines are chosen from hyperbranched polymers comprising at least one amino group and dendrimers bearing at least one amino group, such as PAMAM polyamidoamine dendrimers with an ethylenediamine core and a terminal amine function.

Preferably, the organic amines are chosen from compounds 1d to 37d below, chitosans, salts thereof, optical isomers thereof, geometrical isomers thereof, tautomers thereof, solvates thereof and mixtures thereof:

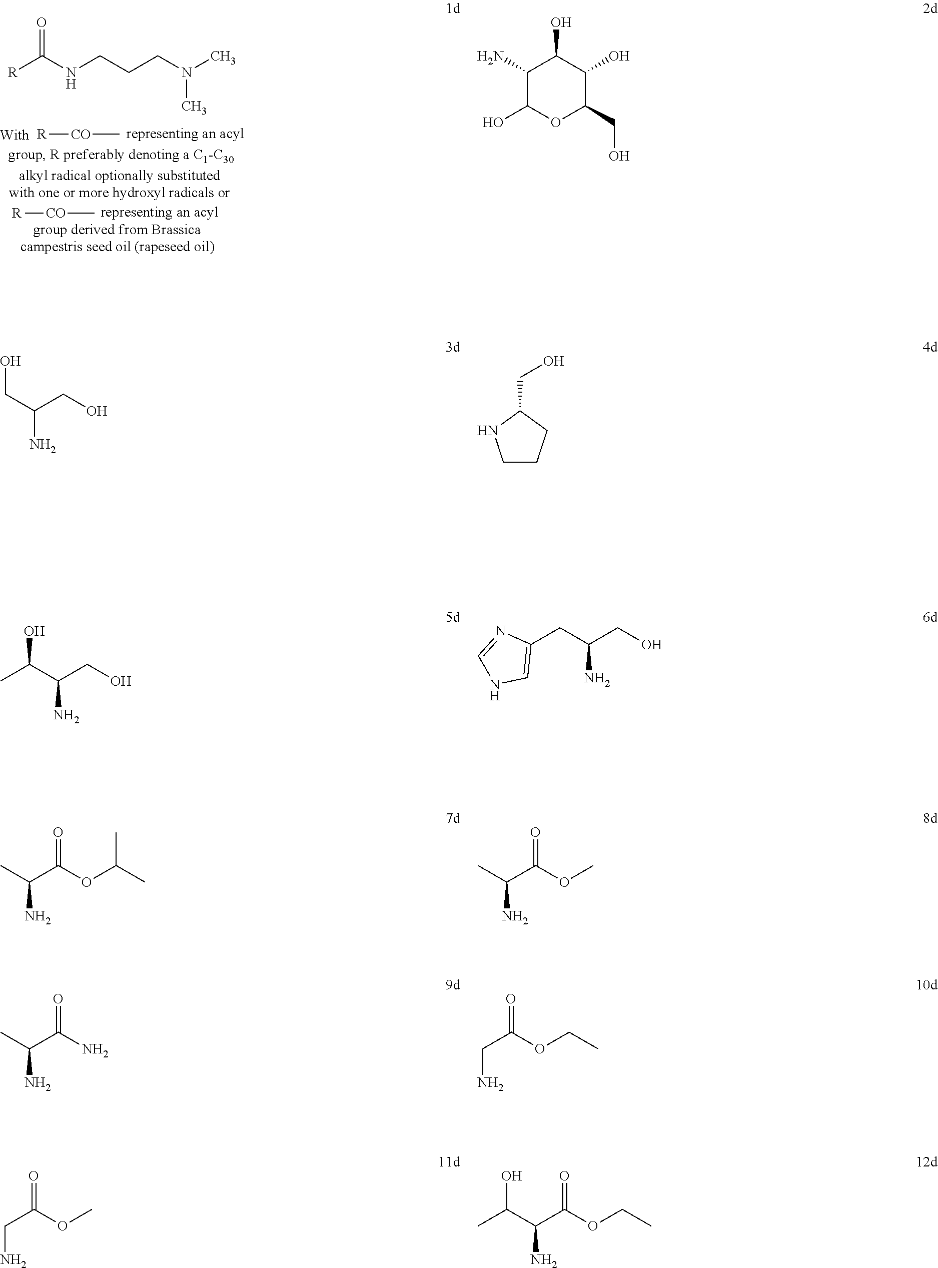

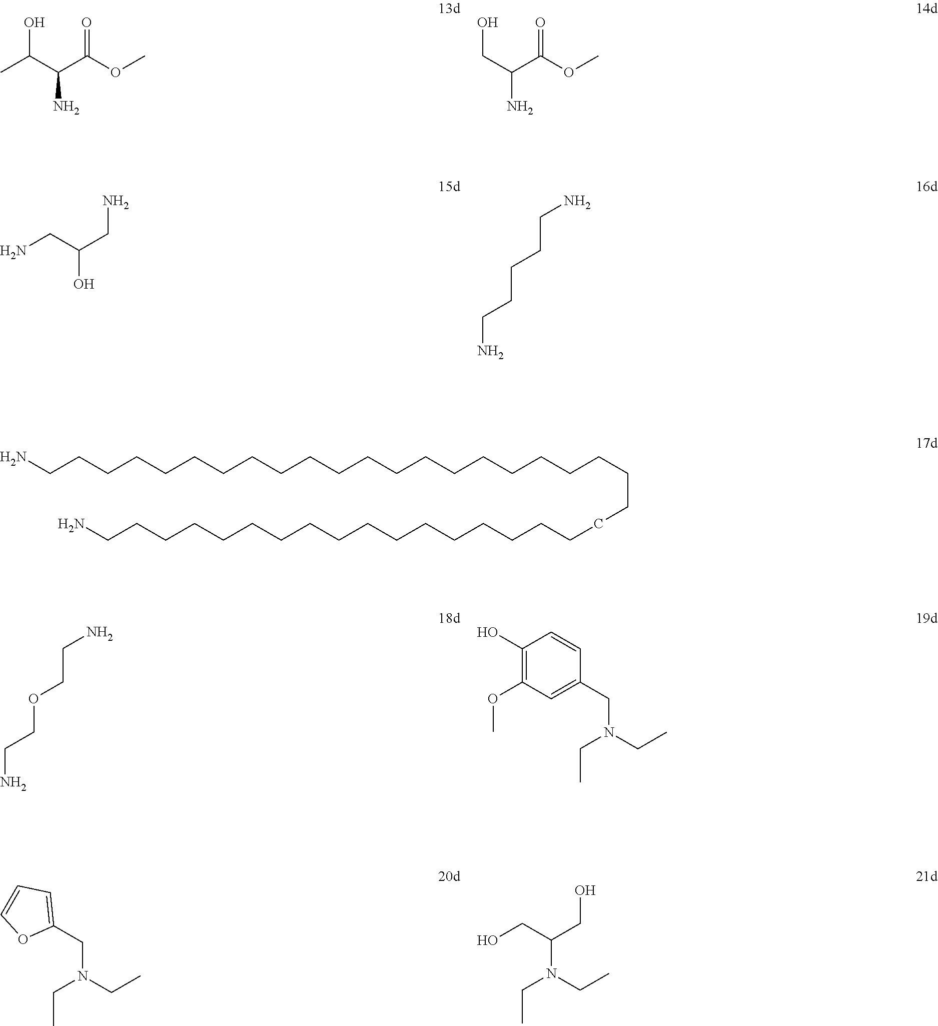

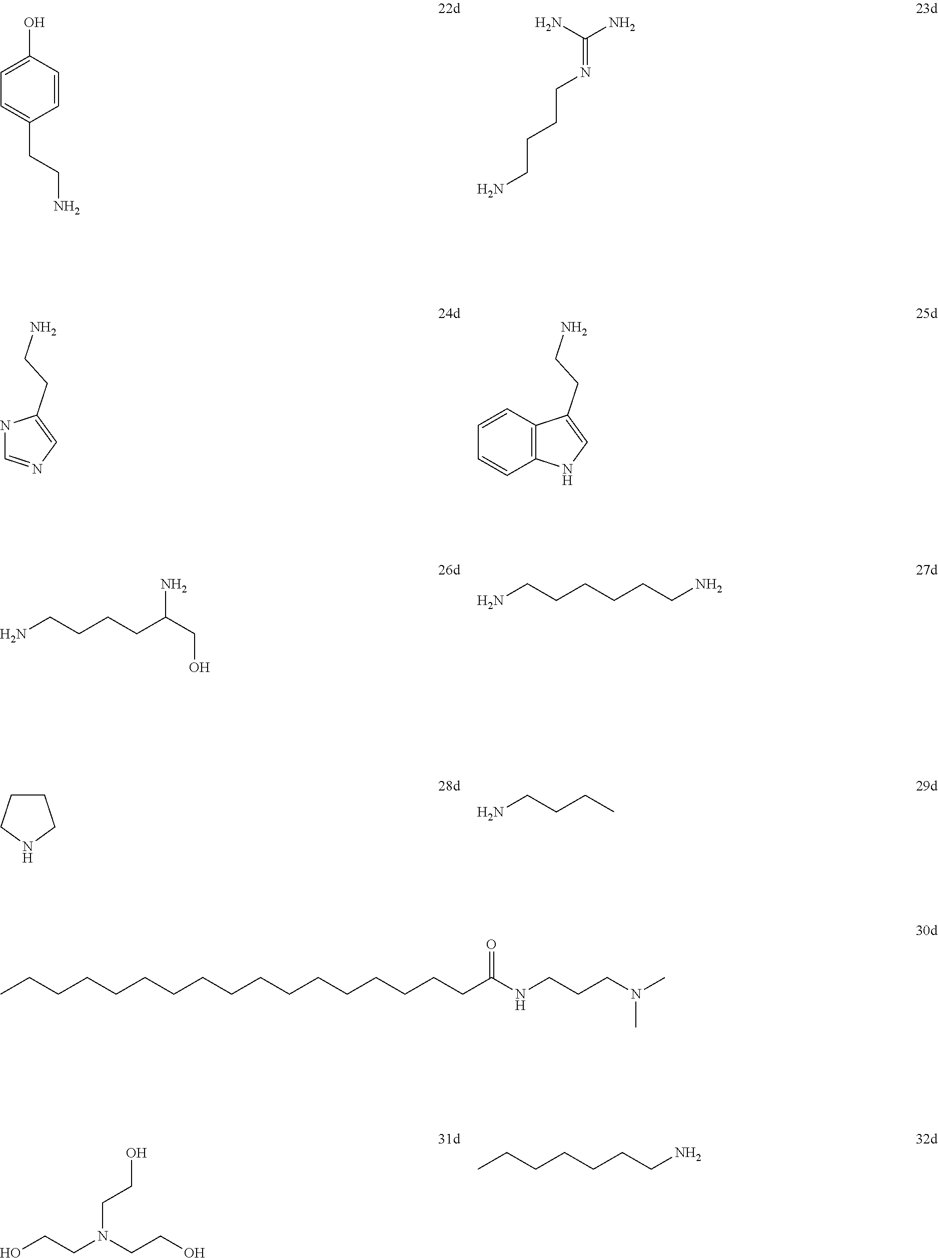

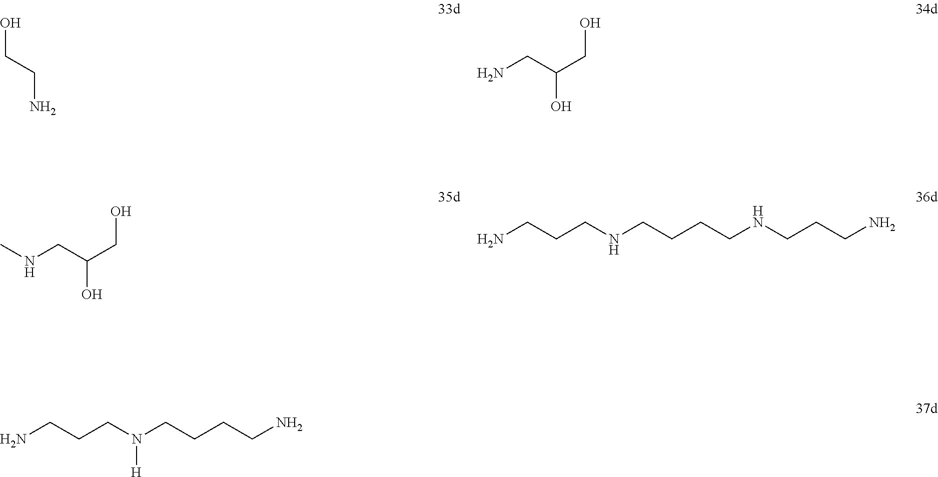

More preferentially, the organic amines are chosen from compounds 1d, 2d, 3d, 22d, 30d, chitosans, salts thereof, optical isomers thereof, geometrical isomers thereof, tautomers thereof, solvates thereof and mixtures thereof.

Even more preferentially, the organic amines are chosen from compound 30d, chitosans, salts thereof, optical isomers thereof, geometrical isomers thereof, tautomers thereof, solvates thereof and mixtures thereof.

Most preferentially, the organic amines are chosen from compound 30d, salts thereof, optical isomers thereof, geometrical isomers thereof, tautomers thereof, solvates thereof and mixtures thereof.

The organic amines are preferably chosen from the compounds of formula (D) hereinbelow, salts thereof, optical isomers thereof, geometrical isomers thereof, tautomers thereof, solvates thereof and mixtures thereof:

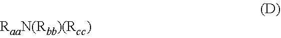

in which:
Rₐₐ, R_{bb} and R_{cc} independently represent:
   a hydrogen atom;
   a C₁-C₄₀ alkyl or C₂-C₄₀ alkenyl group, preferably a C₁-C₄₀ alkyl group, the alkyl or alkenyl group being optionally substituted with at least one group chosen from amino (—NH₂), amido (—CONH₂), imine such as —N═N—NH₂(NH₂), hydroxyl (—OH), ester —O—CO-Ak or —CO—O-Ak, amide —NR—CO-Ak or —CO—NR-Ak1 or with a (hetero)cyclic group optionally substituted with one or more groups chosen from (poly)(hydroxy)alkyl, amino (—NH₂), amine —NR_{dd}(Rₑₑ), hydroxyl (—OH) or imine such as —N═N—NH₂(NH₂), it also being possible for said alkyl or alkenyl group to be interrupted with an oxygen atom, an NR, —OCO—, —CO—O—, —CO—NR—, —NR—CO— or CO group, preferably with an oxygen atom or an —OCO— or —CO—O— ester;
   a heterocyclic group optionally substituted with one or more groups chosen from (poly)(hydroxy)alkyl, amino (—NH₂), amine —NR_{dd}(Rₑₑ), hydroxyl (—OH) or imine such as —N═N—NH₂(NH₂);
Rₐₐ and R_{bb} may form together with the nitrogen atom which bears them an aromatic or non-aromatic heterocyclic group, preferably of 5 or 6 chain members, said heterocyclic group being optionally substituted with one or more groups chosen from (poly)(hydroxy)alkyl, amino (—NH₂), amine —NR_{dd}(Rₑₑ), hydroxy (—OH);
it being understood that Rₐₐ, R_{bb} and R_{cc} cannot simultaneously denote a hydrogen atom;
Ak represents an alkyl or alkenyl group, preferably an alkyl group, said group being optionally substituted with at least one hydroxyl group (—OH), and said group also possibly being interrupted with an oxygen atom, an NR or CO group;
Ak1 represents a hydrogen atom or an alkyl or alkenyl group, preferably alkyl, said alkyl or alkenyl group being optionally substituted with at least one hydroxyl group (—OH) and said alkyl or alkenyl group also possibly being interrupted with an oxygen atom, a group NR or CO;
R_{dd} and Rₑₑ independently represent an alkyl or alkenyl group optionally substituted with at least one group chosen from hydroxyl (—OH), said alkyl or alkenyl group also possibly being interrupted with an oxygen atom, a group NR or CO;
R_{dd} and Rₑₑ may form together with the nitrogen atom which bears them an aromatic or non-aromatic heterocyclic group, preferably of 5 or 6 chain members, said heterocyclic group being optionally substituted with one or more groups chosen from (poly)(hydroxy)alkyl, amino (—NH₂) and hydroxyl (—OH);
R represents a hydrogen atom or a preferably C₁-C₄ alkyl group such as methyl.

According to a first embodiment, the organic amines are preferably chosen from the compounds of formula (D), salts thereof, optical isomers thereof, geometrical isomers thereof, tautomers thereof, solvates thereof and mixtures thereof, in which formula (D) R_{bb} and R_{cc} represent a hydrogen atom.

Preferably, according to this embodiment, Rₐₐ denotes:
a C₁-C₄₀ alkyl or C₂-C₄₀ alkenyl group, preferably a C₁-C₄₀ alkyl group, said alkyl or alkenyl group being optionally substituted with at least one radical chosen from hydroxyl (—OH), amino (—NH₂), amido (—CONH₂), imine such as —N═N—NH₂(NH₂), ester —O—CO-Ak or —CO—O-Ak, or with a heterocyclic radical optionally substituted with one or more radicals chosen from (poly)(hydroxy)alkyl, amino (—NH₂), amine —NR_{dd}(Rₑₑ), hydroxyl (—OH) or imine such as —N═N—NH₂(NH₂), it also being possible for said alkyl or alkenyl group to be interrupted with an oxygen atom, an NR, —OCO—, —CO—O—, —CO—NR—, —NR—CO— or CO group, preferably with an oxygen atom or an —OCO— or —CO—O— ester;
a heterocyclic group optionally substituted with one or more groups chosen from (poly)(hydroxy)alkyl, amino (—NH₂), amine —NR_{dd}(Rₑₑ), hydroxyl (—OH) or imine such as —N═N—NH₂(NH₂), preferably hydroxyl or (poly)(hydroxy)alkyl.

As examples of compounds according to this embodiment, mention may be made of compounds 2d, 3d, 5d to 18d, 22d to 27d, 29d, 32d to 34d, 36d and 37d.

According to a particular variant of this embodiment, the organic amines are preferably chosen from the compounds of formula (D), salts thereof, optical isomers thereof, geometrical isomers thereof, tautomers thereof, solvates thereof and mixtures thereof, in which formula (D) R_{bb} and R_{cc} represent a hydrogen atom, and Rₐₐ represents a heterocyclic group chosen from the sugars as described previously, preferably a monosaccharide or polysaccharide comprising from 2 to 5 saccharide unit(s), preferably from 2 to 3 saccharide unit(s); preferentially, sugar denotes a compound comprising 1 or 2 saccharide unit(s) (monosaccharide or disaccharide), in particular glucosamine or galactosamine.

According to a particular variant of this embodiment, the organic amines are preferably chosen from the compounds of formula (D), salts thereof, optical isomers thereof, geometrical isomers thereof, tautomers thereof, solvates thereof and mixtures thereof, in which formula (D) R_{bb} and R_{cc} represent a hydrogen atom and Rₐₐ represents an alkyl or alkenyl group, preferably a C₁-C₄₀ alkyl substituted with an amino group —NH₂, said alkyl or alkenyl group also being optionally substituted with at least one hydroxyl radical (—OH) and/or interrupted with an oxygen atom.

According to a particular variant of this embodiment, the organic amines are preferably chosen from the compounds of formula (D), salts thereof, optical isomers thereof, geometrical isomers thereof, tautomers thereof, solvates thereof and mixtures thereof, in which formula (D) R_{bb} and R_{cc} represent a hydrogen atom and Rₐₐ represents an alkyl or alkenyl group, preferably a C₁-C₄₀ alkyl optionally substituted with an amino group —NH₂, said alkyl or alkenyl group being interrupted with at least one NR group, wherein R is as defined hereinbefore, preferably representing a hydrogen atom.

According to yet another particular variant of this embodiment, the organic amines are preferably chosen from the compounds of formula (D), salts thereof, optical isomers thereof, geometrical isomers thereof, tautomers thereof, solvates thereof and mixtures thereof, in which formula (D) R_{bb} and R_{cc} denote a hydrogen atom and Rₐₐ represents an alkyl or alkenyl group preferably a C₁-C₁₀ alkyl, more preferentially a C₂-C₁₀ alkyl, optionally substituted with one or more groups chosen from hydroxyl or (hetero)cyclic such as phenyl optionally substituted with OH as described for (D), said alkyl or alkenyl group also being interrupted with an oxygen atom, an ester —OCO— or —CO—O—.

According to another embodiment, the organic amines are preferably chosen from the compounds of formula (D), salts thereof, optical isomers thereof, geometrical isomers thereof, tautomers thereof, solvates thereof and mixtures thereof, in which formula (D) Rₐₐ represents a hydrogen atom and R_{bb} and R_{cc} as defined previously are other than a hydrogen atom; preferably, R_{bb} and R_{cc} independently represent a C₁-C₁₀ alkyl or C₂-C₁₀ alkenyl radical, preferably a C₁-C₁₀ alkyl, more preferentially a C₁-C₆ alkyl, the alkyl or alkenyl group being optionally substituted with one or more hydroxyl radicals (—OH) or Rₐₐ and R_{bb} form together with the nitrogen atom which bears them an aromatic or non-aromatic, preferably non-aromatic, 5- or 6-membered heterocyclic group, the heterocyclic group being optionally substituted with one or more groups chosen from C₁-C₄ (poly)(hydroxy)alkyl and hydroxyl (—OH).

According to another embodiment, the organic amines are preferably chosen from the compounds of formula (D), salts thereof, optical isomers thereof, geometrical isomers thereof, tautomers thereof, solvates thereof and mixtures thereof, in which formula (D) Rₐₐ, R_{bb} and R_{cc} as defined previously are other than a hydrogen atom; preferably, Rₐₐ, R_{bb} and R_{cc} independently represent a C₁-C₁₀ alkyl or C₂-C₁₀ alkenyl group, preferably a C₁-C₁₀ alkyl, more preferentially a C₁-C₆ alkyl, said alkyl or alkenyl group being optionally substituted with one or more groups chosen from hydroxyl (—OH), aromatic or non-aromatic, preferably aromatic, 5- or 6-membered (hetero)cyclic group, said (hetero)cyclic group being optionally substituted with one or more groups chosen from C₁-C₄ (poly)(hydroxy)alkyl and C₁-C₄ alkoxy. Preferably, Rₐₐ and R_{bb} independently represent a C₁-C₁₀ alkyl group, more preferentially a C₁-C₆ alkyl group optionally substituted with one or more hydroxyl (—OH) groups, and R_{cc} represents an aromatic or non-aromatic, preferably aromatic 5- or 6-membered (hetero)cyclic group, said (hetero)cyclic group being optionally substituted with one or more groups chosen from C₁-C₄ (poly)hydroxyalkyl, hydroxyl (—OH) and C₁-C₄ alkoxy.

When they are present in the composition, the organic amines d) are preferably present in the composition in a content ranging from 0.01% to 50%, more preferentially ranging from 0.1% to 30%, even more preferentially ranging from 0.2% to 15% by weight relative to the total weight of the composition.

### Compounds e)

Preferably, the compounds e) are chosen from compounds 1e to 18e below, optical isomers thereof, geometrical isomers thereof, tautomers thereof, solvates thereof and mixtures thereof:

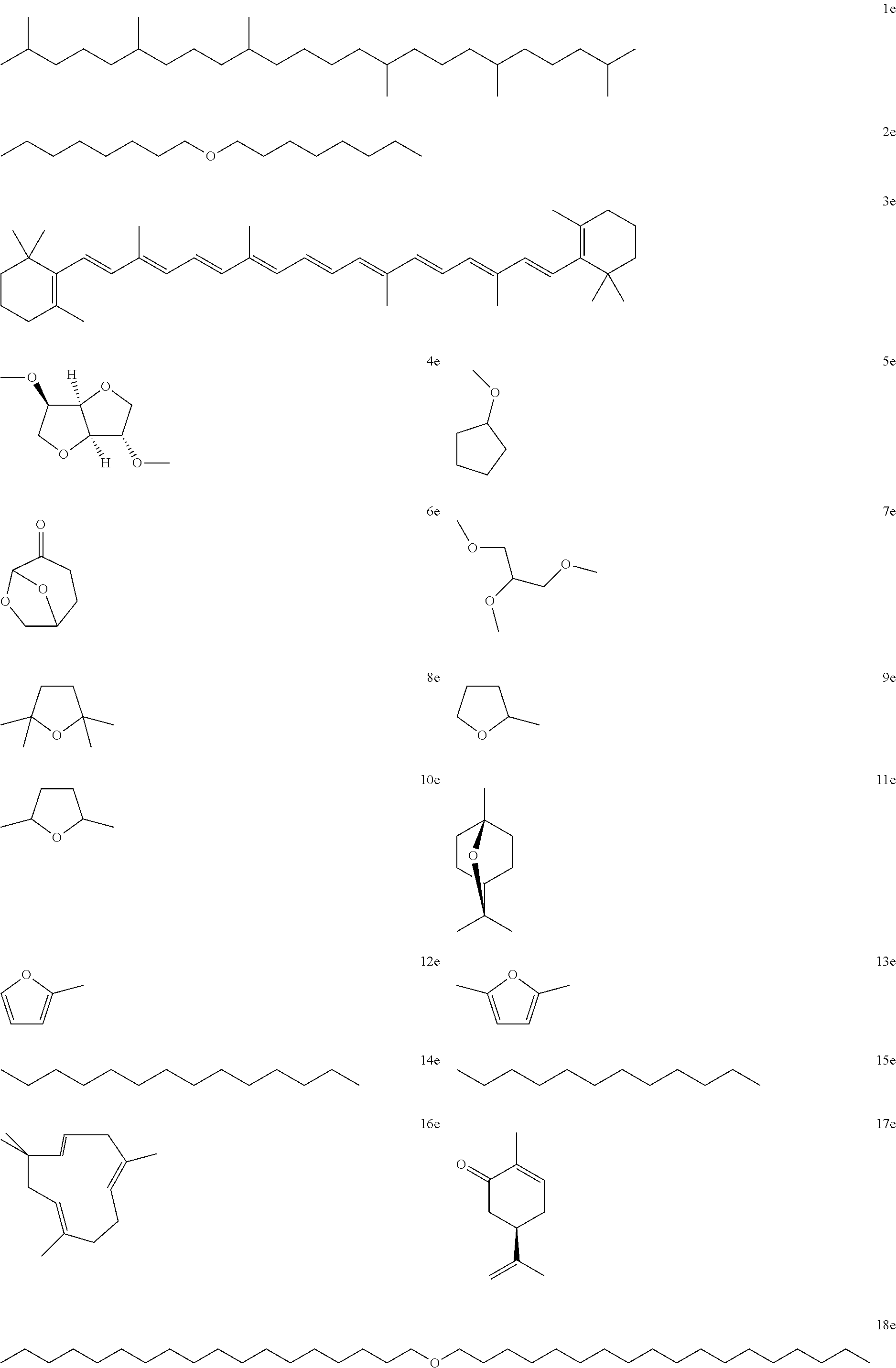

More preferentially, the compounds e) are chosen from compounds 1e, 2e, 4e and 18e, optical isomers thereof, geometrical isomers thereof, tautomers thereof, solvates thereof and mixtures thereof.

Even more preferentially, the compounds e) are chosen from compound 2e, solvates thereof and mixtures thereof.

Preferably, the compounds e) do not include an amine, ester, amide, alcohol, imine or acid function.

The compounds e) are preferably chosen from linear or branched C₇-C₄₀ alkanes such as compounds 1e, 14e, 15e and 18e, ethers of formula (E) hereinbelow:

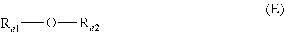

in which:
Rₑ₁ and Rₑ₂ independently represent a linear or branched C₁-C₂₀ alkyl group optionally interrupted with one or more oxygen atoms or a 5- or 6-membered carbocycle such as cyclohexyl or cyclopentyl;

Rₑ₁ and Rₑ₂ may form, with the oxygen atom which bears them, an aromatic or non-aromatic heterocyclic group, preferably with 5 or 6 chain members, said heterocyclic group being optionally substituted notably with one or more groups chosen from (C₁-C₄)alkyl.

When they are present in the composition, the compounds e) are preferably present in the composition in a content ranging from 0.01% to 50%, more preferentially ranging from 0.1% to 30%, even more preferentially ranging from 0.02% to 15% by weight relative to the total weight of the composition.

The compound(s) iv′) are preferably present in the composition in a total content ranging from 0.01% to 50%, more preferentially ranging from 0.1% to 30%, even more preferentially ranging from 0.2% to 15% by weight relative to the total weight of the composition.

The alcohol(s) iv) and the compound(s) iv′) are present in the composition in a total content by weight of iv) and iv′) preferably ranging from 0.01% to 70% by weight, more preferentially ranging from 1% to 60% by weight, even more preferentially ranging from 10% to 60% by weight relative to the total weight of the composition.

The composition preferably comprises a total content of magnesium carbonate of less than 5% by weight, more preferentially of less than 1% by weight, even more preferentially of less than 0.1% by weight, most preferentially of less than 0.01% by weight and better still of less than 0.001% by weight.

According to one particularly preferred embodiment, the composition is free of magnesium carbonate.

The composition preferably comprises a total content of persulfates of less than 1% by weight, more preferentially less than 0.1% by weight, even more preferentially less than 0.01% by weight, most preferentially less than 0.001% by weight.

According to a particularly preferred embodiment, the composition is free of persulfates.

According to a particular embodiment, the composition also comprises v) one or more dyes preferably chosen from direct dyes, oxidation dyes and mixtures thereof.

When they are present, the dye(s) are preferably present in a total content ranging from 0.001% to 10% by weight, preferably from 0.01% to 4% by weight, and more preferentially from 0.1% to 1% by weight, relative to the total weight of the composition.

### Oxidation Dyes

The oxidation dyes are generally chosen from one or more oxidation bases optionally combined with one or more coupling agents (also known as couplers).

### Oxidation Bases

The composition may optionally comprise one or more oxidation bases advantageously chosen from those conventionally used in the dyeing of keratin fibres.

By way of example, the oxidation bases are chosen from para-phenylenediamines, bis(phenyl)alkylenediamines, para-aminophenols ortho-aminophenols and heterocyclic bases, and the corresponding addition salts.

Among the para-phenylenediamines that may be mentioned are, for example, para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-methoxymethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the corresponding addition salts with an acid.

Among the para-phenylenediamines mentioned above, para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the corresponding addition salts with an acid, are particularly preferred.

Among the bis(phenyl)alkylenediamines that may be mentioned, for example, are N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis((3-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the corresponding addition salts.

Among the para-aminophenols that are mentioned are, for example, para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the corresponding addition salts with an acid.

Among the ortho-aminophenols that may be mentioned are, for example, 2-aminophenol, 2-amino-5-methylphenol, 2-amino-6-methylphenol and 5-acetamido-2-aminophenol, and the corresponding addition salts.

Among the heterocyclic bases that may be mentioned, for example, are pyridine, pyrimidine and pyrazole derivatives.

Among the pyridine derivatives that may be mentioned are the compounds described, for example, in patents GB 1 026 978 and GB 1 153 196, for example 2,5-diaminopyridine, 2-(4-methoxyphenyl)amino-3-aminopyridine and 3,4-diaminopyridine, and the corresponding addition salts.

Other pyridine oxidation bases that are useful in the present invention are the 3-aminopyrazolo[1,5-a]pyridine oxidation bases or the corresponding addition salts described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-(morpholin-4-yl)pyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-(morpholin-4-yl)pyrazolo[1,5-a]pyrid-3-ylamine, pyrazolo[1,5-a]pyridine-3,5-diamine, 5-(morpholin-4-yl)pyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-β-hydroxyethoxy-3-aminopyrazolo[1,5-a]pyridine and 2-(4-dimethylpiperazinium-1-yl)-3-aminopyrazolo[1,5-a]pyridine; and the corresponding addition salts.

More particularly, the oxidation bases that are useful in the present invention are chosen from 3-aminopyrazolo[1,5-a]pyridines preferably substituted on carbon atom 2 with:
a) a (di)(C₁-C₆)(alkyl)amino group, said alkyl group possibly being substituted with at least one hydroxyl, amino or imidazolium group;
b) an optionally cationic 5- to 7-membered heterocycloalkyl group comprising from 1 to 3 heteroatoms, optionally substituted with one or more (C₁-C₆)alkyl groups such as a di(C₁-C₄)alkylpiperazinium group; or
c) a (C₁-C₆)alkoxy group optionally substituted with one or more hydroxyl groups, such as a β-hydroxyalkoxy group, and the corresponding addition salts.

Among the pyrimidine derivatives that may be mentioned are the compounds described, for example, in patents DE 2359399; JP 88-169571; JP 05-63124; EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and the addition salts thereof and the tautomeric forms thereof, when a tautomeric equilibrium exists.

Among the pyrazole derivatives that may be mentioned are the compounds described in patents DE 3843892 and DE 4133957 and patent applications WO 94/08969, WO 94/08970, FR-A-2 733 749 and DE 195 43 988, for instance 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the corresponding addition salts. Use may also be made of 4,5-diamino-1-(β-methoxyethyl)pyrazole.

A 4,5-diaminopyrazole will preferably be used and even more preferentially 4,5-diamino-1-(β-hydroxyethyl)pyrazole and/or a corresponding salt.

The pyrazole derivatives that may also be mentioned include diamino-N,N-dihydropyrazolopyrazolones and in particular those described in patent application FR-A-2 886 136, such as the following compounds and the corresponding addition salts: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-bis(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one and 2,3-diamino-6-hydroxy-6,7-dihydro-1H,5H-pyrazolo[1,2-a]pyrazol-1-one.

Use will preferably be made of 2,3-diamino-6,7-dihydro-1H,5H-pyrazolo[1,2-a]pyrazol-1-one and/or a corresponding salt.

Heterocyclic bases that will preferably be used are 4,5-diamino-1-(β-hydroxyethyl)pyrazole and/or 2,3-diamino-6,7-dihydro-1H,5H-pyrazolo[1,2-a]pyrazol-1-one and/or a corresponding salt.

### Coupling Agents

The composition may optionally comprise one or more coupling agents advantageously chosen from those conventionally used in the dyeing of keratin fibres.

Among these couplers, mention may be made in particular of meta-phenylenediamines, meta-aminophenols, meta-diphenols, naphthalene-based coupling agents and heterocyclic coupling agents, and also the corresponding addition salts.

Mention may be made, for example, of 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene, 6-hydroxyindoline, 2,6-dihydroxy-4-methylpyridine, 1-H-3-methylpyrazol-5-one, 1-phenyl-3-methylpyrazol-5-one, 2,6-dimethylpyrazolo[1,5-b]-1,2,4-triazole, 2,6-dimethyl[3,2-c]-1,2,4-triazole and 6-methylpyrazolo[1,5-a]benzimidazole, 2-methyl-5-aminophenol, 5-N-(β-hydroxyethyl)amino-2-methylphenol, 3-aminophenol and 3-amino-2-chloro-6-methylphenol, the corresponding addition salts with an acid and the corresponding mixtures.

In general, the addition salts of the oxidation bases and couplers that may be used in the context of the invention are chosen in particular from the addition salts with an acid such as the hydrochlorides, hydrobromides, sulfates, citrates, succinates, tartrates, lactates, tosylates, benzenesulfonates, phosphates and acetates.

The oxidation base(s) each advantageously represent from 0.001% to 10% by weight relative to the total weight of the composition, and preferably from 0.005% to 5% by weight relative to the total weight of the composition.

The coupling agent(s), if they are present, each advantageously represent from 0.001% to 10% by weight relative to the total weight of the composition, and preferably from 0.005% to 5% by weight relative to the total weight of the composition.

### Direct Dyes

The composition may also comprise one or more direct dyes.

The direct dyes may be neutral, cationic or anionic direct dyes, preferably neutral or cationic direct dyes.

The direct dyes may be neutral, cationic or anionic direct dyes chosen from: acridines; acridones; anthanthrones; anthrapyrimidines; anthraquinones; azines; (poly)azos or azos, hydrazono or hydrazones, in particular arylhydrazones; azomethines; benzanthrones; benzimidazoles; benzimidazolones; benzindoles; benzoxazoles; benzopyrans; benzothiazoles; benzoquinones; bis-isoindolines; carboxanilides; coumarins; cyanines, such as (di)azacarbocyanines, (di)azahemicyanines, hemicyanines or tetraazacarbocyanines; (di)azines; bis-azines; (di)oxazines; (di)thiazines; (di)phenylamines; (di)phenylmethanes; (di)ketopyrrolopyrroles; flavonoids, such as flavanthrones and flavones; fluorindines; formazans; indamines; indanthrones; indigoids, thioindigoids and pseudoindigoids; indophenols; indoanilines; isoindolines; isoindolinones; isoviolanthrones; lactones; (poly)methines, such as dimethines of stilbene or styryl types; naphthalimides; naphthanilides; naphtholactams; naphthoquinones; nitro, notably nitro(hetero)aromatics; oxadiazoles; oxazines; perilones; perinones; perylenes; phenazines; phenoxazines; phenothiazines; phthalocyanines; polyenes/carotenoids; porphyrins; pyranthrones; pyrazolanthrones; pyrazolones; pyrimidinoanthrones; pyronines; quinacridones; quinolines; quinophthalones; squaranes; tetrazolines; thiazines; thiopyronines; triarylmethanes or xanthenes and natural direct dyes. Preferably, the direct dyes are chosen from anthraquinones, (poly)azos, azomethines and stilbenes, more preferentially from anthraquinones.

The direct dyes may be chosen in particular from neutral, cationic or anionic nitrobenzene direct dyes, neutral, cationic or anionic azo direct dyes, neutral, cationic or anionic tetraazapentamethine dyes, cationic or anionic quinone dyes and in particular neutral, cationic or anionic anthraquinone dyes, neutral, cationic or anionic azine direct dyes, neutral, cationic or anionic triarylmethane direct dyes, neutral, cationic or anionic azomethine direct dyes and natural direct dyes. Preferably, the direct dyes are chosen from neutral or anionic anthraquinone dyes and stilbenes.

As neutral, anionic or cationic direct dyes that may be used in the present invention, mention may be made of the following dyes: acridines; acridones; anthanthrones; anthrapyrimidines; anthraquinones; azines; (poly)azos, hydrazono or hydrazones, in particular arylhydrazones; azomethines; benzanthrones; benzimidazoles; benzimidazolones; benzindoles; benzoxazoles; benzopyrans; benzothiazoles; benzoquinones; bisazines; bis-isoindolines; carboxanilides; coumarins; cyanines, such as azacarbocyanines, diazacarbocyanines, diazahemicyanines, hemicyanines or tetraazacarbocyanines; diazines; diketopyrrolopyrroles; dioxazines; diphenylamines; diphenylmethanes; dithiazines; flavonoids, such as flavanthrones and flavones; fluorindines; formazans; indamines; indanthrones; indigoids and pseudoindigoids; indophenols; indoanilines; isoindolines; isoindolinones; isoviolanthrones; lactones; (poly)methines, such as dimethines of stilbene or styryl types; naphthalimides; naphthanilides; naphtholactams; naphthoquinones; nitro, notably nitro(hetero)aromatics; oxadiazoles; oxazines; perilones; perinones; perylenes; phenazines; phenoxazine; phenothiazines; phthalocyanine; polyenes/carotenoids; porphyrins; pyranthrones; pyrazolanthrones; pyrazolones; pyrimidinoanthrones; pyronines; quinacridones; quinolines; quinophthalones; squaranes; tetrazoles; thiazines; thioindigo; thiopyronines; triarylmethanes or xanthenes.

### Neutral Direct Dyes

The direct dyes may be neutral direct dyes, preferably chosen from the hydrazono dyes of formulae (IIIa) and (III′a), the azo and styryl dyes (IVa), the diazo and distyryl dyes (IV′a) and (IV″a), the anthraquinone dyes (Va) and the azomethine dyes (VIa) and (VI′a) below, and mixtures thereof:

| | | | |
|---|---|---|---|
| Ar″—C(R^{a})═N—N(R^{b})—Ar | Ar″—N(R^{a})—N═C(R^{b})—Ar | Ar—T═T′—Ar″ | Ar″—T═T′—Ar′—T′═T—Ar |
| (IIIa) | (III′a) | (IVa) | (IV′a) |

| | |
|---|---|
| 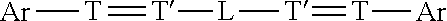 | 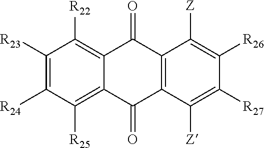 |
| (IV″a) | (Va) |

| | |
|---|---|
| 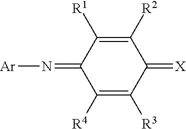 | 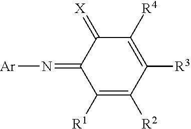 |
| (VIa) | (VI′a) |

in which formulae (IIIa), (III′a), (IVa), (IV′a), (IV″a), (Va), (VIa) and (VI′a):
Ar represents an aryl group, such as phenyl or naphthyl, substituted with at least one electron-donating group such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino, or Ar represents a julolidine group;
Ar′ represents an optionally substituted divalent (hetero)arylene group such as phenylene, particularly para-phenylene, or naphthalene, which is optionally substituted, preferably with one or more (C₁-C₈)alkyl, hydroxyl or (C₁-C₈)alkoxy groups;
Ar″ represents a (hetero)aryl group, which is optionally substituted, preferably with at least i) an electron-withdrawing group such as nitro, nitroso, —C(X)—X′—R′ or ii) a (di)(C₁-C₆)(alkyl)amino group, iii) hydroxyl, iv) (C₁-C₆)alkoxy; (hetero)aryl is particularly chosen from imidazolyl, triazolyl, indolyl or pyridyl or phenyl optionally substituted with at least one group chosen from nitro, nitroso and amino, preferably substituted in the para position of the phenyl group;
X, X′ and X″, which may be identical or different, represent an oxygen or sulfur atom, or a group NR″, preferably an oxygen atom;
R¹, R², R³ and R⁴, which may be identical or different, represent a hydrogen or halogen atom, or a group chosen from hydroxyl, thiol, (C₁-C₄)alkyl, (C₁-C₄)alkoxy, (di)(C₁-C₄)(alkyl)amino, nitro and nitroso;
R′ and R″ represent a (C₁-C₄)alkyl group;
R^{a} and R^{b}, which may be identical or different, represent a hydrogen atom or a (C₁-C₈)alkyl group, which is optionally substituted, preferably with a hydroxyl group;

or, as a variant, the substituent R^{a} with a substituent of Ar″ and/or R^{b} with a substituent of Ar and/or R^{a} with R^{b} form, together with the atoms that bear them, a (hetero)cycloalkyl; in particular, R^{a} and R^{b} represent a hydrogen atom or a (C₁-C₄)alkyl group, which is optionally substituted with a hydroxyl group;
T and T′, which may be identical or different, represent a group C(R^{a}) or N, preferably N; and
L represents a divalent group -ALK-, —C(X)-ALK-, -ALK—C(X)— or —C(X)-ALK-C(X′)— with ALK representing a linear or branched (C₁-C₆)alkylene group, such as methylene, and X and X′ as defined previously;
R₂₂, R₂₃, R₂₄, R₂₅, R₂₆ and R₂₇, which may be identical or different, represent a hydrogen or halogen atom, or a group chosen from:
(C₁-C₆)alkyl;
hydroxyl, mercapto;
(C₁-C₆)alkoxy, (C₁-C₆)alkylthio;
aryloxy or arylthio;
aryl(C₁-C₆)(alkyl)amino;
(di)(C₁-C₆)(alkyl)amino;
(di)(hydroxy(C₁-C₆)alkyl)amino;
Z′ represents a hydrogen atom or a group NR₂₈R₂₉ with R₂₈ and R₂₉, which may be identical or different, representing a hydrogen atom or a group chosen from:
(C₁-C₆)alkyl;
polyhydroxy(C₁-C₆)alkyl such as hydroxyethyl;
aryl optionally substituted with one or more group(s), particularly i) (C₁-C₆)alkyl; iii) R^{o}—C(X)—X′—, R^{o}—X′—C(X)—, R^{o}—X′—C(X)—X″— with R^{o} representing a (C₁-C₆)alkyl group and X, X′ and X″ as defined previously; iv) a sulfonate;
cycloalkyl; notably cyclohexyl;
Z represents a group chosen from hydroxyl and NR′₂₈R′₂₉ with R′₂₈ and R′₂₉, which may be identical or different, representing the same atoms or groups as R₂₈ and R₂₉ as defined previously.

The direct dyes of formula (IV″a) are preferably of formula (IV′″a)

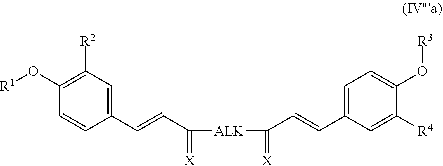

in which formula (IV′″a):
R¹ and R³, which may be identical or different, preferably identical, represent a hydrogen atom, a (C₁-C₄)alkyl group such as methyl or a sugar such as glucosyl, preferably a hydrogen atom;
R² and R⁴, which may be identical or different, preferably identical, represent a hydrogen atom, a (C₁-C₄)alkyl or (C₁-C₄)alkoxy group or an —O-sugar group such as —O-glucosyl, preferably (C₁-C₄)alkoxy; such as methoxy;
X, which may be identical or different, preferably identical, represents an oxygen or sulfur atom or N—R with R representing a hydrogen atom or a group, preferably an oxygen atom;
ALK represents a (C₁-C₄) alkylene group such as methylene or ethylene, preferably methylene.

The direct dyes of formula (IV″a) may be derived from curcumin, demethoxycurcumin and bis-demethoxycurcumin.

Preferably, the direct dyes are chosen from the direct dyes of formulae (IV″a) and (IV′″a) and mixtures thereof as defined previously.

According to a particularly preferred embodiment, the direct dyes are neutral direct dyes chosen from the following compounds (A) to (G) and mixtures thereof:

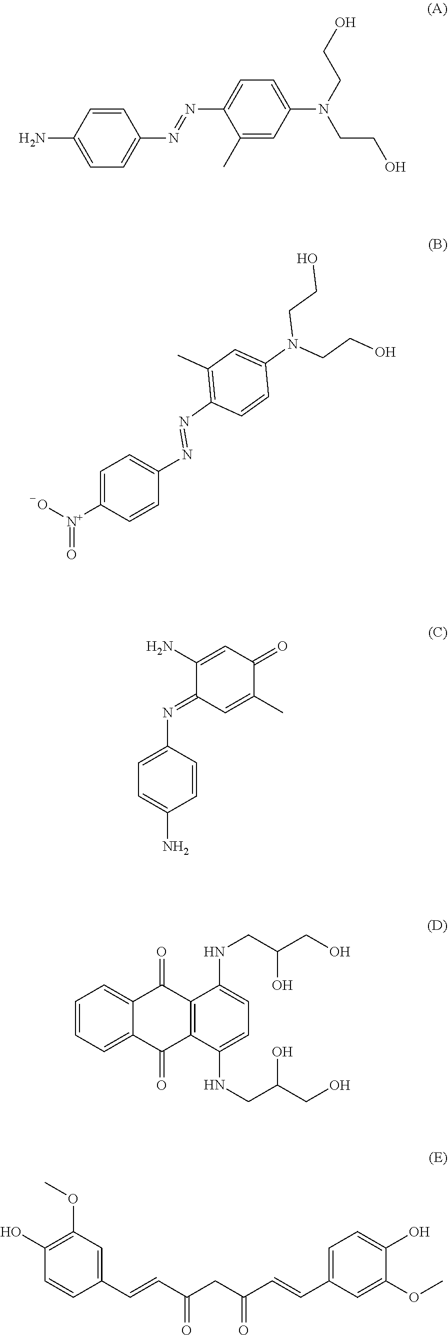

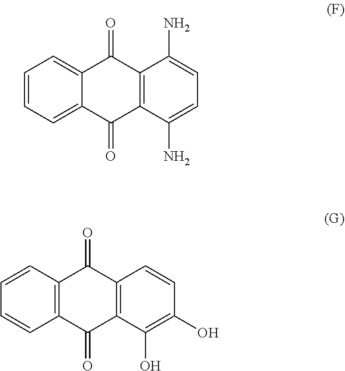 [SMILES: Nc1ccc(N)c2c1C(=O)c1ccccc1C2=O ; O=C1c2ccccc2C(=O)c2c1ccc(O)c2O]

preferably from the compounds (E), (F) and (G) and mixtures thereof, more preferentially from the compounds (E) and (G) and mixtures thereof.

### Cationic Direct Dyes

The direct dyes may be chosen from direct dyes which are cationic or commonly referred to as “basic dyes” for their affinity with acidic substances notably including in their structure at least one endocyclic or exocyclic cationic or cationizable group.

As cationic azo dyes that can be used in the present invention, mention may be made particularly of the cationic dyes described in Kirk-Othmer's Encyclopedia of Chemical Technology, “Dyes, Azo”, J. Wiley & Sons, updated on Apr. 19, 2010.

Mention may also be made of the cationic azo dyes described in patent applications WO 95/15144, WO 95/01772 and EP 714 954.

Mention may also be made of the cationic azo dyes described in the Colour Index International 3rd Edition, notably of the following compounds: Basic Red 22; Basic Red 76; Basic Yellow 57; Basic Brown 16; Basic Brown 17.

Among the cationic quinone dyes, those mentioned in the Colour Index International 3rd Edition, are suitable for use and, among these, mention may be made, inter alia, of the following dyes: Basic Blue 22; Basic Blue 99.

Among the azine dyes that are suitable for use, mention may be made of those listed in the Colour Index International 3rd Edition, for example the following dyes: Basic Blue 17, Basic Red 2.

Among the cationic triarylmethane dyes that can be used according to the invention, mention may be made, in addition to those listed in the Color Index International, 3rd edition, of the following dyes: Basic Green 1, Basic Violet 3, Basic Violet 14, Basic Blue 7, Basic Blue 26.

Mention may also be made of the direct dyes in the documents U.S. Pat. No. 5,888,252, EP 1 133 975, WO 03/029359, EP 860 636, WO 95/01772, WO 95/15144 and EP 714 954. Mention may also be made of those listed in the encyclopaedia “The Chemistry of Synthetic Dyes” by K. Venkataraman, 1952, Academic Press, volumes 1 to 7, in the “Kirk-Othmer Encyclopaedia of Chemical Technology”, in the chapter “Dyes and Dye Intermediates”, 1993, Wiley and Sons, and in various chapters of “Ullmann's Encyclopaedia of Industrial Chemistry”, 7th edition, Wiley and Sons.

Preferably, the cationic direct dyes are chosen from those resulting from dyes of azo and hydrazono type.

The cationic direct dyes may be cationic azo dyes, as described in EP 850 636, FR 2 788 433, EP 920 856, WO 99/48465, FR 2 757 385, EP 850 637, EP 918 053, WO 97/44004, FR 2 570 946, FR 2 285 851, DE 2 538 363, FR 2 189 006, FR 1 560 664, FR 1 540 423, FR 1 567 219, FR 1 516 943, FR 1 221 122, DE 4220388, DE 4 137 005, WO 01/66646, U.S. Pat. No. 5,708,151, WO 95/01772, WO 515 144, GB 1 195 386, U.S. Pat. Nos. 3,524,842, 5,879,413, EP 1 062 940, EP 1 133 976, GB 738 585, DE 2 527 638, FR 2 275 462, GB 1974-27645, Acta Histochem. (1978), 61(1), 48-52; Tsitologiya (1968), 10(3), 403-5; Zh. Obshch. Khim. (1970), 40(1), 195-202; Ann. Chim. (Rome) (1975), 65(5-6), 305-14; Journal of the Chinese Chemical Society (Taipei) (1998), 45(1), 209-211; Rev. Roum. Chim. (1988), 33(4), 377-83; Text. Res. J. (1984), 54(2), 105-7; Chim. Ind. (Milan) (1974), 56(9), 600-3; Khim. Tekhnol. (1979), 22(5), 548-53; Ger. Monatsh. Chem. (1975), 106(3), 643-8; MRL Bull. Res. Dev. (1992), 6(2), 21-7; Lihua Jianyan, Huaxue Fence (1993), 29(4), 233-4; Dyes Pigm. (1992), 19(1), 69-79; Dyes Pigm. (1989), 11(3), 163-72.

Preferably, the cationic direct dyes comprise a quaternary ammonium group; more preferentially, the cationic charge is endocyclic. These cationic groups are, for example, a cationic group:
bearing a (di/tri)(C₁-C₈)alkylammonium exocyclic charge, or
having an endocyclic charge, such as comprising a cationic heteroaryl group chosen from: acridinium, benzimidazolium, benzobistriazolium, benzopyrazolium, benzopyridazinium, benzoquinolium, benzothiazolium, benzotriazolium, benzoxazolium, bipyridinium, bis-tetrazolium, dihydrothiazolium, imidazopyridinium, imidazolium, indolium, isoquinolium, naphthoimidazolium, naphthoxazolium, naphthopyrazolium, oxadiazolium, oxazolium, oxazolopyridinium, oxonium, phenazinium, phenoxazolium, pyrazinium, pyrazolium, pyrazoyltriazolium, pyridinium, pyridinoimidazolium, pyrrolium, pyrylium, quinolium, tetrazolium, thiadiazolium, thiazolium, thiazolopyridinium, thiazoylimidazolium, thiopyrylium, triazolium or xanthylium.

Mention may be made of the cationic hydrazono direct dyes of formulae (IIb) and (IIIb) and the cationic azo direct dyes of formulae (IVb) and (Vb) below:

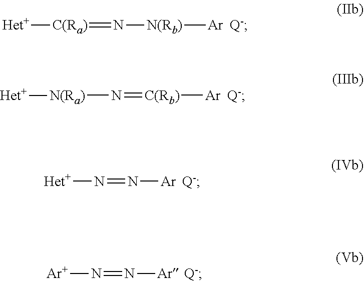

in which formulae (IIb) to (Vb):
Het⁺ represents a cationic heteroaryl group, preferentially bearing an endocyclic cationic charge, such as imidazolium, indolium or pyridinium, which is optionally substituted, preferentially with at least one (C₁-C₈)alkyl group such as methyl;
Ar⁺ represents an aryl group, such as phenyl or naphthyl, bearing an exocyclic cationic charge, preferentially ammonium, particularly tri(C₁-C₈)alkylammonium, such as trimethylammonium;
Ar represents an aryl group, notably phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar″ represents an optionally substituted (hetero)aryl group, such as phenyl or pyrazolyl, which are optionally substituted, preferentially with one or more (C₁-C₈)alkyl, hydroxyl, (di)(C₁-C₈)(alkyl)amino, (C₁-C₈)alkoxy or phenyl groups;
Rₐ and R_{b}, which may be identical or different, represent a hydrogen atom or a (C₁-C₈)alkyl group which is optionally substituted, preferentially with a hydroxyl group;
or else the substituent Rₐ with a substituent of Het⁺ and/or R_{b} with a substituent of Ar and/or Rₐ with R_{b} form, together with the atoms which bear them, a (hetero)cycloalkyl; in particular, Rₐ and R_{b} represent a hydrogen atom or a (C₁-C₄)alkyl group optionally substituted with a hydroxyl group;
Q⁻ represents an anionic counterion such as a halide, an alkyl sulfate or an alkylsulfonate.

In particular, mention may be made of the azo and hydrazono direct dyes bearing an endocyclic cationic charge of formulae (IIb) to (Vb) as defined previously. More particularly, mention may be made of the cationic direct dyes of formulae (IIb) to (Vb) bearing an endocyclic cationic charge described in patent applications WO 95/15144, WO 95/01772 and EP 714954.

Preferably, mention may be made of the following direct dyes:

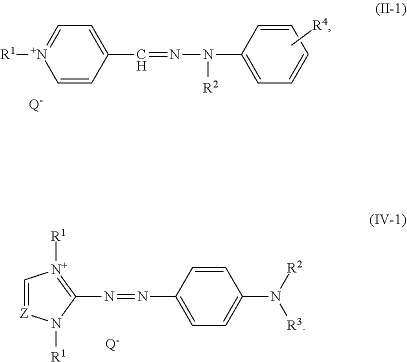

in which formulae (II-1) and (IV-1):
R¹ represents a (C₁-C₄)alkyl group such as methyl;
R² and R³, which may be identical or different, represent a hydrogen atom or a (C₁-C₄)alkyl group, such as methyl;
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; in particular, R⁴ is a hydrogen atom;
Z represents a CH group or a nitrogen atom, preferentially CH,
Q⁻ is an anionic counterion as defined previously, in particular a halide, such as chloride, or an alkyl sulfate, such as methyl sulfate or mesyl.

Particularly, the dyes of formulae (II-1) and (IV-1) are chosen from Basic Red 51, Basic Yellow 87 and Basic Orange 31 or derivatives thereof:

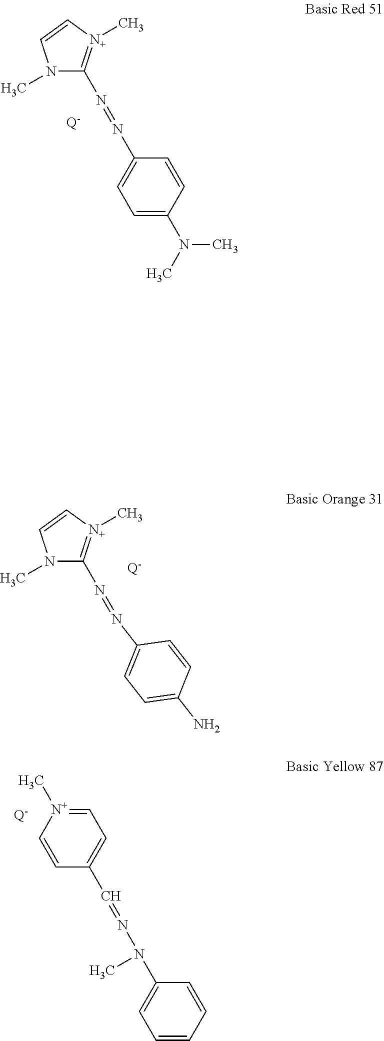

with Q′ being an anionic counterion as defined previously, in particular a halide, such as chloride, or an alkyl sulfate, such as methyl sulfate or mesyl.

### Fluorescent Dyes

The direct dyes may be chosen from fluorescent direct dyes.

As examples of fluorescent dyes that may be used in the present invention, mention may be made of neutral, anionic or cationic dyes chosen from the following dyes: acridines, acridones, benzanthrones, benzimidazoles, benzimidazolones, benzindoles, benzoxazoles, benzopyrans, benzothiazoles, coumarins, difluoro{2-[(2H-pyrrol-2-ylidene-kN)methyl]-1H-pyrrolato-kN}borons (BODIPY®), diketopyrrolopyrroles, fluorindines, (poly)methines (notably cyanines and styryls/hemicyanines), naphthalimides, naphthanilides, naphthylamines (such as dansyls), oxadiazoles, oxazines, perilones, perinones, perylenes, polyenes/carotenoids, squaranes, stilbenes, xanthenes.

Mention may also be made of the fluorescent dyes described in EP 1 133 975, WO 03/029 359, EP 860 636, WO 95/01772, WO 95/15144 and EP 714 954 and those listed in the encyclopaedia “The Chemistry of Synthetic Dyes” by K. Venkataraman, 1952, Academic Press, volumes 1 to 7, in the “Kirk-Othmer Encyclopaedia of Chemical Technology”, in the chapter “Dyes and Dye Intermediates”, 1993, Wiley and Sons, and in various chapters of “Ullmann's Encyclopaedia of Industrial Chemistry”, 7th edition, Wiley and Sons, and in the handbook—“A Guide to Fluorescent Probes and Labeling Technologies”, 10th Ed., Molecular Probes/Invitrogen—Oregon 2005, circulated on the Internet or in the preceding printed editions.

According to a preferred variant, the fluorescent dye(s) are cationic polymethines and comprise at least one quaternary ammonium group, such as those of formula (Vb) below:

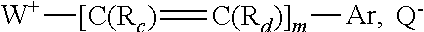

in which formula (Vb):
W⁺ represents a cationic heterocyclic or heteroaryl group, particularly comprising a quaternary ammonium optionally substituted with one or more (C₁-C₈)alkyl groups, optionally substituted notably with one or more hydroxyl groups;
Ar representing an aryl group such as phenyl or naphthyl, optionally substituted preferentially with i) one or more halogen atoms such as chlorine or fluorine; ii) one or more (C₁-C₈)alkyl and preferably (C₁-C₄)alkyl groups such as methyl; iii) one or more hydroxyl groups; iv) one or more (C₁-C₈)alkoxy groups such as methoxy; v) one or more hydroxy(C₁-C₈)alkyl groups such as hydroxyethyl, vi) one or more amino or (di)(C₁-C₈)alkylamino groups, preferably with the C₁-C₄ alkyl part optionally substituted with one or more hydroxyl groups, such as (di)hydroxyethylamino, vii) with one or more acylamino groups; viii) one or more heterocycloalkyl groups such as piperazinyl, piperidyl or 5- or 6-membered heteroaryl such as pyrrolidinyl, pyridyl and imidazolinyl;
m′ represents an integer ranging from 1 to 4; preferably, m′ is equal to 1 or 2; more preferentially, m′=1;
R_{c} and R_{d}, which may be identical or different, represent a hydrogen atom or an optionally substituted (C₁-C₈)alkyl group, preferably an optionally substituted (C₁-C₄)alkyl group, or alternatively R_{c} contiguous with W⁺ and/or R_{d} contiguous with Ar form, with the atoms which bear them, a (hetero)cycloalkyl; in particular, R_{c} is contiguous with W⁺ and they form a (hetero)cycloalkyl, such as cyclohexyl;
Q⁻ is an anionic counterion as defined previously.

### Anionic Dyes

The direct dyes may be chosen from anionic direct dyes or dyes commonly referred to as “acidic” direct dyes on account of their affinity with alkaline substances.

The term “anionic direct dye” means any direct dye including in its structure at least one CO₂R or SO₃R substituent with R denoting a hydrogen atom or a cation originating from a metal or an amine, or an ammonium ion. The anionic dyes may be chosen from direct nitro acid dyes, azo acid dyes, azine acid dyes, triarylmethane acid dyes, indoamine acid dyes, anthraquinone acid dyes, indigoid dyes and natural acid dyes.

Preferably, the anionic direct dyes are anthraquinone acid dyes.

The direct dyes may be anionic direct dyes preferably chosen from the dyes of formulae (III), (III′), (IV), (IV′), (V), (V′), (VI), (VI′), (VII), (VIII), (IX) and (X) below, and mixtures thereof:

### a) The Diaryl Anionic Azo Dyes of Formula (III) or (III′):

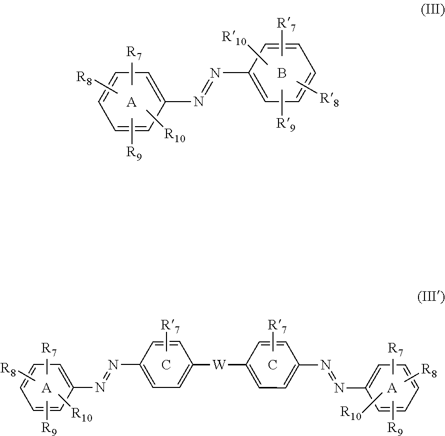

in which formulae (III) and (III′):
R₇, R₈, R₉, R₁₀, R′₇, R′₈, R′₉ and R′₁₀, which may be identical or different, represent a hydrogen atom or a group chosen from:
(C₁-C₆)alkyl;
(C₁-C₆)alkoxy, (C₁-C₆)alkylthio;
hydroxyl, mercapto;
nitro, nitroso;
R^{o}—C(X)—X′—, R^{o}—X′—C(X)—, R^{o}—X′—C(X)—X″— with R^{o} representing a hydrogen atom or a (C₁-C₆)alkyl or aryl group such as phenyl; X, X′ and X″, which may be identical or different, representing an oxygen or sulfur atom, or NR with R representing a hydrogen atom or a (C₁-C₆)alkyl group;
(O)₂S(O⁻)—, M⁺ with M⁺ representing a hydrogen atom or a cationic counterion;
(O)CO⁻—, M⁺ with M⁺ as defined previously;
R″—S(O)₂—, with R″ representing a hydrogen atom, an alkyl group, or an aryl, (di)(C₁-C₆)(alkyl)amino, or aryl(C₁-C₆)(alkyl)amino group; preferentially a phenylamino or phenyl group;
R′″—S(O)₂—X′— with R′″ representing a (C₁-C₆)alkyl group or an aryl group which is optionally substituted, X′ as defined previously;
(di)(C₁-C₆)(alkyl)amino;
aryl(C₁-C₆)(alkyl)amino optionally substituted with one or more groups chosen from i) nitro; ii) nitroso; iii) (O)₂S(O⁻)—, M⁺ and iv) (C₁-C₆)alkoxy with M⁺ as defined previously;
optionally substituted heteroaryl; preferentially a benzothiazolyl group;
cycloalkyl; notably cyclohexyl;
Ar—N═N— with Ar representing an optionally substituted aryl group; preferentially a phenyl optionally substituted with one or more alkyl, (O)₂S(O⁻), M⁺ or phenylamino groups;
or else two contiguous groups R₇ with R₈ or R₈ with R₉ or R₉ with R₁₀ together form a fused benzo group A′; and R′₇ with R′₈ or R′₈ with R′₉ or R′₉ with R′₁₀ together form a fused benzo group B′; with A′ and B′ optionally substituted with one or more group(s) chosen from i) nitro; ii) nitroso; iii) (O)₂S(O⁻)—, M⁺; iv) hydroxyl; v) mercapto; vi) (di)(alkyl)amino; vii) R^{o}—C(X)—X′—; viii) R^{o}—X′—C(X)—; ix) R^{o}—X′—C(X)—X″—; x) Ar—N═N— and xi) optionally substituted aryl(C₁-C₆)(alkyl)amino; with M⁺, R^{o}, X, X′, X″ and Ar as defined previously;
W represents a sigma bond σ, an oxygen or sulfur atom, or a divalent group i) —NR— with R as defined previously, or ii) methylene —C(Rₐ)(R_{b})— with Rₐ and R_{b}, which may be identical or different, representing a hydrogen atom or an aryl group, or alternatively Rₐ and R_{b} together form, with the carbon atom that bears them, a spiro cycloalkyl; preferentially, W represents a sulfur atom or Rₐ and R_{b} together form a cyclohexyl; it being understood that the formulae (III) and (III′) comprise, on one of the rings A, A′, B, B′ or C:
at least one group (O)₂S(O⁻)—, M′⁺ with M′⁺ representing a cationic counterion; or
at least one group (O)CO⁻—, M′⁺ with M′⁺ representing a cationic counterion;

preferably at least one sodium sulfonate group.

As examples of dyes of formula (III), mention may be made of: Acid Red 1, Acid Red 4, Acid Red 13, Acid Red 14, Acid Red 18, Acid Red 27, Acid Red 28, Acid Red 32, Acid Red 33, Acid Red 35, Acid Red 37, Acid Red 40, Acid Red 41, Acid Red 42, Acid Red 44, Pigment Red 57, Acid Red 68, Acid Red 73, Acid Red 135, Acid Red 138, Acid Red 184, Food Red 1, Food Red 13, Acid Orange 6, Acid Orange 7, Acid Orange 10, Acid Orange 19, Acid Orange 20, Acid Orange 24, Yellow 6, Acid Yellow 9, Acid Yellow 36, Acid Yellow 199, Food Yellow 3, Acid Violet 7, Acid Violet 14, Acid Blue 113, Acid Blue 117, Acid Black 1, Acid Brown 4, Acid Brown 20, Acid Black 26, Acid Black 52, Food Black 1, Food Black 2, Food Yellow 3 or Sunset Yellow;

and as examples of dyes of formula (III′), mention may be made of: Acid Red 111, Acid Red 134, Acid Yellow 38;

### b) The Pyrazolone Anionic Azo Dyes of Formula (IV) or (IV′):

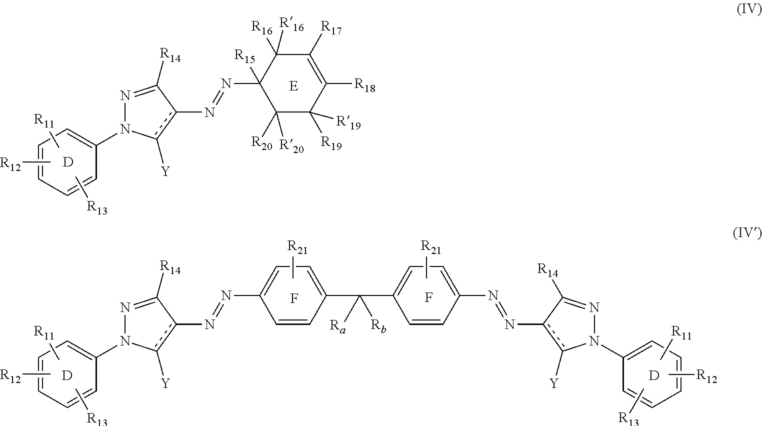

in which formulae (IV) and (IV′):
R₁₁, R₁₂ and R₁₃, which may be identical or different, represent a hydrogen or halogen atom, a (C₁-C₆)alkyl group or —(O)₂S(O⁻), M⁺ with M⁺ as defined previously;
R₁₄ represents a hydrogen atom, a (C₁-C₆)alkyl group or a —C(O)O⁻, M⁺ group with M⁺ as defined previously;
R₁₅ represents a hydrogen atom;
R₁₆ represents an oxo group, in which case R′₁₆ is absent, or alternatively R₁₅ with R₁₆ together form a double bond;
R₁₇ and R₁₈, which may be identical or different, represent a hydrogen atom or a group chosen from:
(O)₂S(O⁻)—, M⁺ with M⁺ as defined previously;
Ar—O—S(O)₂— with Ar representing an optionally substituted aryl group; preferentially a phenyl optionally substituted with one or more alkyl groups;
R₁₉ and R₂₀ together form either a double bond, or a benzo group D′, which is optionally substituted;
R′₁₆, R′₁₉ and R′₂₀, which may be identical or different, represent a hydrogen atom or a (C₁-C₆)alkyl group, or a hydroxyl group;
R₂₁ represents a hydrogen atom or a (C₁-C₆)alkyl or (C₁-C₆)alkoxy group;
Rₐ and R_{b}, which may be identical or different, are as defined previously; preferentially, Rₐ represents a hydrogen atom and R_{b} represents an aryl group such as phenyl;
Y represents either a hydroxyl group or an oxo group;
[symbol] represents a single bond when Y is an oxo group; and represents a double bond when Y represents a hydroxyl group;

it being understood that the formulae (IV) and (IV′) comprise, on one of the rings D or E:
at least one group (O)₂S(O⁻)—, M′⁺ with M′⁺ representing a cationic counterion; or
at least one group (O)CO⁻—, M′⁺ with M′⁺ representing a cationic counterion;

preferably at least one sodium sulfonate group.

As examples of dyes of formula (IV), mention may be made of: Acid Red 195, Acid Yellow 23, Acid Yellow 27, Acid Yellow 76, and as examples of dyes of formula (IV′), mention may be made of: Acid Yellow 17;

### c) The Anthraquinone Dyes of Formula (V) or (V′):

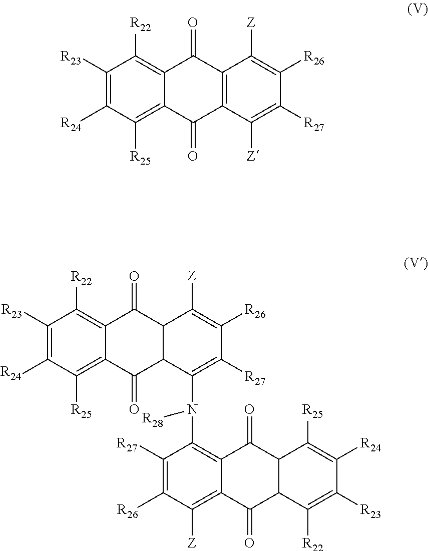

in which formulae (V) and (V′):
R₂₂, R₂₃, R₂₄, R₂₅, R₂₆ and R₂₇, which may be identical or different, represent a hydrogen or halogen atom, or a group chosen from:
(C₁-C₆)alkyl;
hydroxyl, mercapto;
(C₁-C₆)alkoxy, (C₁-C₆)alkylthio;
optionally substituted aryloxy or arylthio, preferentially substituted with one or more groups chosen from alkyl and (O)₂S(O⁻)—, M⁺ with M⁺ as defined previously;
aryl(C₁-C₆)(alkyl)amino optionally substituted with one or more groups chosen from alkyl and (O)₂S(O⁻)—, M⁺ with M⁺ as defined previously;
(di)(C₁-C₆)(alkyl)amino;
(di)(hydroxy)(C₁-C₆)(alkyl)amino;
(O)₂S(O⁻)—, M⁺ with M⁺ as defined previously;
Z′ represents a hydrogen atom or a group NR₂₈R₂₉ with R₂₈ and R₂₉, which may be identical or different, representing a hydrogen atom or a group chosen from:
(C₁-C₆)alkyl;
polyhydroxy(C₁-C₆)alkyl such as hydroxyethyl;
aryl optionally substituted with one or more group(s), particularly i) (C₁-C₆)alkyl such as methyl, n-dodecyl, n-butyl; ii) (O)₂S(O⁻)—, M⁺ with M⁺ as defined previously; iii) R^{o}—C(X)—X′—, R^{o}—X′—C(X)—, R^{o}—X′—C(X)—X″— with R^{o}, X, X′ and X″ as defined previously; preferentially R^{o} represents a (C₁-C₆)alkyl group;
cycloalkyl, notably cyclohexyl;
Z represents a group chosen from hydroxyl and NR′₂₈R′₂₉ with R′₂₈ and R′₂₉, which may be identical or different, representing the same atoms or groups as R₂₈ and R₂₉ as defined previously;

it being understood that the formulae (V) and (V′) comprise:
at least one group (O)₂S(O⁻)—, M′⁺ with M′⁺ representing a cationic counterion; or
at least one group (O)CO⁻—, M′⁺ with M′⁺ representing a cationic counterion;

preferably at least one sodium sulfonate group.

As examples of dyes of formula (V), mention may be made of: Acid Blue 25, Acid Blue 43, Acid Blue 62, Acid Blue 78, Acid Blue 129, Acid Blue 138, Acid Blue 140, Acid Blue 251, Acid Green 25, Acid Green 41, Acid Violet 42, Acid Violet 43, Mordant Red 3; EXT Violet No. 2;

and as examples of dyes of formula (V′), mention may be made of: Acid Black 48;

### d) The Nitro Dyes of Formula (VI) or (VI′):

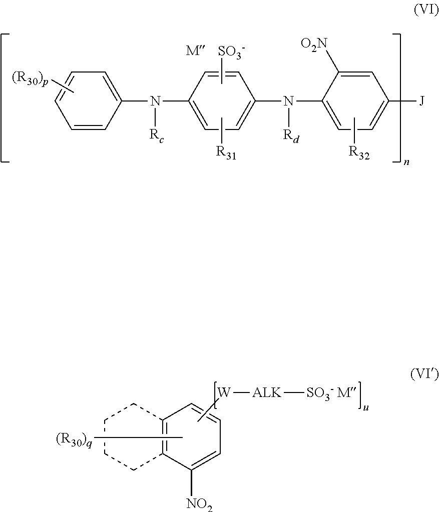

in which formulae (VI) and (VI′):
R₃₀, R₃₁ and R₃₂, which may be identical or different, represent a hydrogen or halogen atom, or a group chosen from:
(C₁-C₆)alkyl;
(C₁-C₆)alkoxy optionally substituted with one or more hydroxyl group(s) or (C₁-C₆)alkylthio optionally substituted with one or more hydroxyl group(s);
hydroxyl, mercapto;
nitro, nitroso;
polyhalo(C₁-C₆)alkyl;
R^{o}—C(X)—X′—, R^{o}—X′—C(X)—, R^{o}—X′—C(X)—X″— with R^{o}, X, X′ and X″ as defined previously;
(O)₂S(O⁻)—, M⁺ with M⁺ as defined previously;
(O)CO⁻—, M⁺ with M⁺ as defined previously;
(di)(C₁-C₆)(alkyl)amino;
(di)(hydroxy)(C₁-C₆)(alkyl)amino;
heterocycloalkyl such as piperidino, piperazino or morpholino;

in particular, R₃₀, R₃₁ and R₃₂ represent a hydrogen atom;
R_{c} and R_{d}, which may be identical or different, represent a hydrogen atom or a (C₁-C₆)alkyl group;
W is as defined previously; W particularly represents an N(H)— group;
ALK represents a linear or branched divalent C₁-C₆ alkylene group; in particular, ALK represents a —CH₂—CH₂— group;
n is 1 or 2;
p represents an integer ranging from 1 to 5;
q represents an integer ranging from 1 to 4;
u is 0 or 1;
when n is 1, J represents a nitro or nitroso group; particularly nitro;
when n is 2, J represents an oxygen or sulfur atom, or a divalent group —S(O)ₘ— with m representing an integer 1 or 2; preferentially, J represents an —SO₂— group;
M″ represents a hydrogen atom or a cationic counterion;

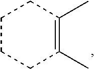

which may be present or absent, represents a benzo group optionally substituted with one or more groups R₃₀ as defined previously;

it being understood that the formulae (VI) and (VI′) comprise:
at least one group (O)₂S(O⁻)—, M′⁺ with M′⁺ representing a cationic counterion; or
at least one group (O)CO⁻—, M′⁺ with M′⁺ representing a cationic counterion;

preferably at least one sodium sulfonate group.

As examples of dyes of formula (VI), mention may be made of: Acid Brown 13 and Acid Orange 3; as examples of dyes of formula (VI′), mention may be made of: Acid Yellow 1, sodium salt of 2,4-dinitro-1-naphthol-7-sulfonic acid, 2-piperidino-5-nitrobenzenesulfonic acid, 2-(4′-N,N-(2″-hydroxyethyl)amino-2′-nitro)anilineethanesulfonic acid, 4-β-hydroxyethylamino-3-nitrobenzenesulfonic acid; Ext. D&C Yellow 7;

### e) The Triarylmethane Dyes of Formula (VII):

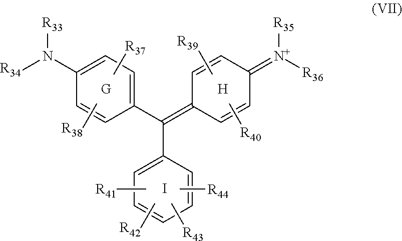

in which formula (VII):
R₃₃, R₃₄, R₃₅ and R₃₆, which may be identical or different, represent a hydrogen atom or a group chosen from (C₁-C₆)alkyl, optionally substituted aryl and optionally substituted aryl(C₁-C₆)alkyl; particularly a (C₁-C₆)alkyl group and benzyl optionally substituted with a group (O)ₘS(O⁻)—, M⁺ with M⁺ and m as defined previously;
R₃₇, R₃₈, R₃₉, R₄₀, R₄₁, R₄₂, R₄₃ and R₄₄, which may be identical or different, represent a hydrogen atom or a group chosen from:
(C₁-C₆)alkyl;
(C₁-C₆)alkoxy, (C₁-C₆)alkylthio;
(di)(C₁-C₆)(alkyl)amino;
hydroxyl, mercapto;
nitro, nitroso;
R^{o}—C(X)—X′—, R^{o}—X′—C(X)—, R^{o}—X′—C(X)—X″— with R^{o} representing a hydrogen atom, or an alkyl or aryl group; X, X′ and X″, which may be identical or different, representing an oxygen or sulfur atom, or NR with R representing a hydrogen atom or a (C₁-C₆)alkyl group;
(O)₂S(O⁻)—, M⁺ with M⁺ representing a hydrogen atom or a cationic counterion;
(O)CO⁻—, M⁺ with M⁺ as defined previously;
or else two contiguous groups R₄₁ with R₄₂ or R₄₂ with R₄₃ or R₄₃ with R₄₄ together form a fused benzo group optionally substituted with one or more group(s) chosen from i) nitro; ii) nitroso; iii) (O)₂S(O⁻)—, M⁺; iv) hydroxyl; v) mercapto; vi) (di)(C₁-C₆)(alkyl)amino; vii) R^{o}—C(X)—X′—; viii) R^{o}—X′—C(X)—; ix) R^{o}—X′—C(X)—X″—; with M⁺, R^{o}, X, X′ and X″ as defined previously;

particularly, R₃₇ to R₄₀ represent a hydrogen atom, and R₄₁ to R₄₄, which may be identical or different, represent a hydroxyl or (O)₂S(O⁻)—, M⁺ group with M⁺ as defined previously; and when R₄₃ with R₄₄ together form a benzo group, it is preferentially substituted with an (O)₂S(O⁻)— group;

it being understood that at least one of the rings G, H or I comprises:
at least one group (O)₂S(O⁻)—, M′⁺ with M′⁺ representing a cationic counterion; or
at least one group (O)CO⁻—, M′⁺ with M′⁺ representing a cationic counterion;

preferably at least one sodium sulfonate group.

As examples of dyes of formula (VII), mention may be made of: Acid Blue 1; Acid Blue 3; Acid Blue 7, Acid Blue 9; Acid Violet 49; Acid Green 3; Acid Green 5 and Acid Green 50.

### f) The Xanthene-Based Dyes of Formula (VIII):

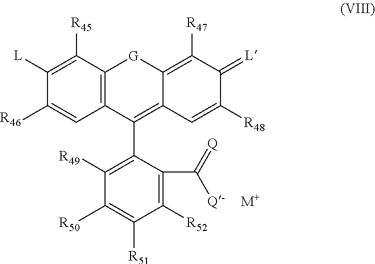

in which formula (VIII):
R₄₅, R₄₆, R₄₇ and R₄₈, which may be identical or different, represent a hydrogen or halogen atom;
R₄₉, R₅₀, R₅₁ and R₅₂, which may be identical or different, represent a hydrogen or halogen atom, or a group chosen from:
(C₁-C₆)alkyl;
(C₁-C₆)alkoxy, (C₁-C₆)alkylthio;
hydroxyl, mercapto;
nitro, nitroso;
(O)₂S(O⁻)—, M⁺ with M⁺ representing a hydrogen atom or a cationic counterion;
(O)CO⁻—, M⁺ with M⁺ as defined previously;

particularly, R₅₃, R₅₄, R₅₅ and R₄₈ represent a hydrogen or halogen atom;
G represents an oxygen or sulfur atom or a group NRₑ with Rₑ as defined previously; particularly, G represents an oxygen atom;
L represents an alkoxide O⁻, M⁺; a thioalkoxide S⁻, M⁺ or a group NR_{f}, with R_{f} representing a hydrogen atom or a (C₁-C₆)alkyl group, and M⁺ as defined previously; M⁺ is particularly sodium or potassium;
L′ represents an oxygen or sulfur atom or an ammonium group: N⁺R_{f}R_{g}, with R_{f} and R_{g}, which may be identical or different, representing a hydrogen atom, a (C₁-C₆)alkyl group or an optionally substituted aryl group; L′ represents particularly an oxygen atom or a phenylamino group optionally substituted with one or more alkyl or (O)ₘS(O⁻)—, M⁺ group(s) with m and M⁺ as defined previously;
Q and Q′, which may be identical or different, represent an oxygen or sulfur atom; particularly Q and Q′ represent an oxygen atom;
M⁺ is as defined previously.

As examples of dyes of formula (VIII), mention may be made of: Acid Yellow 73; Acid Red 51; Acid Red 52; Acid Red 87; Acid Red 92; Acid Red 95; Acid Violet 9;

### g) The Indole-Based Dyes of Formula (IX):

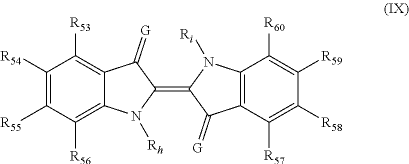

in which formula (IX):
R₅₃, R₅₄, R₅₅, R₅₆, R₅₇, R₅₈, R₅₉ and R₆₀, which may be identical or different, represent a hydrogen atom or a group chosen from:
(C₁-C₆)alkyl;
(C₁-C₆)alkoxy, (C₁-C₆)alkylthio;
hydroxyl, mercapto;
nitro, nitroso;
R^{o}—C(X)—X′—, R^{o}—X′—C(X)—, R^{o}—X′—C(X)—X″— with R^{o} representing a hydrogen atom, or an alkyl or aryl group; X, X′ and X″, which may be identical or different, representing an oxygen or sulfur atom, or NR with R representing a hydrogen atom or a (C₁-C₆)alkyl group;
(O)₂S(O⁻)—, M⁺ with M⁺ representing a hydrogen atom or a cationic counterion;
(O)CO⁻—, M⁺ with M⁺ as defined previously;
G represents an oxygen or sulfur atom or a group NRₑ with Rₑ as defined previously; particularly, G represents an oxygen atom;
Rᵢ and Rₕ, which may be identical or different, represent a hydrogen atom or a (C₁-C₆)alkyl group;

it being understood that formula (IX) comprises:
at least one group (O)₂S(O⁻)—, M′⁺ with M′⁺ representing a cationic counterion; or
at least one group (O)CO⁻—, M′⁺ with M′⁺ representing a cationic counterion;

preferably at least one sodium sulfonate group.

As examples of dyes of formula (IX), mention may be made of: Acid Blue 74.

### h) The Quinoline-Based Dyes of Formula (X):

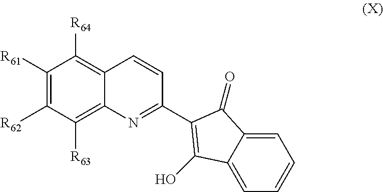

in which formula (X):
R₆₁ represents a hydrogen or halogen atom or a (C₁-C₆)alkyl group;
R₆₂, R₆₃ and R₆₄, which may be identical or different, represent a hydrogen atom or a group (O)₂S(O⁻)—, M⁺ with M⁺ representing a hydrogen atom or a cationic counterion; or else R₆₁ with R₆₂, or R₆₁ with R₆₄, together form a benzo group optionally substituted with one or more groups (O)₂S(O⁻)—, M⁺ with M⁺ representing a hydrogen atom or a cationic counterion;

it being understood that formula (X) comprises at least one group (O)₂S(O⁻)—, M′⁺ with M′⁺ representing a cationic counterion, preferably at least one sodium sulfonate group.

As examples of dyes of formula (X), mention may be made of: Acid Yellow 2, Acid Yellow 3 and Acid Yellow 5.

More particularly, the dyes of formulae (III) to (VIII) that are useful in the invention are chosen from: Acid Red 87 (VIII) (C.I. 45380); Sodium salt of 2,4-dinitro-1-naphthol-7-sulfonic acid (VI′) (C.I. 10316); Acid Orange 3 (VI) (C.I. 10383); Acid Yellow 9/Food Yellow 2 (III) (C.I. 13015); Direct Red 45/Food Red 13 (III) (C.I. 14780); Acid Black 52 (III) (C.I. 13711); Acid Yellow 36 (III) (C.I. 13065); sodium salt of 1-hydroxy-2-(2′,4′-xylyl-5-sulfonatoazo)naphthalene-4-sulfonic acid/Food Red 1 (III) (C.I. 14700); Acid Red 14/Food Red 3/Mordant Blue 79 (III) (C.I. 14720); Sodium salt of 4-hydroxy-3-[(2-methoxy-5-nitrophenyl)diaza]-6-(phenylamino)naphthalene-2-sulfonic acid/Acid Brown 4 (III) (C. I. 14805); Acid Orange 7/Pigment Orange 17/Solvent Orange 49 (III) (C.I. 15510); Food Yellow 3/Pigment Yellow 104 (III) (C.I. 15985); Acid Red 27/Food Red 9 (III) (C.I. 16185); Acid Orange 10/Food Orange 4 (III) (C.I. 16230); Acid Red 44 (III) (C.I. 16250); Acid Red 33/Food Red 12 (III) (C.I. 17200); Acid Red 184 (III) (C.I. 15685); Acid Violet 3 (III) (C.I. 19125); Sodium salt of 1-hydroxy-2-(4′-acetamidophenylazo)-8-acetamidonaphthalene-3,6-disulfonic acid/Acid Violet 7/Food Red 11 (III) (C.I. 18055); Acid Red 135 (III) (C.I. 18130); Acid Yellow 27 (IV) (C.I. 19130); Acid Yellow 23/Food Yellow 4 (IV) (C.I. 19140); 4′-(Sulfonato-2″,4″-dimethyl)bis(2,6-phenylazo)-1,3-dihydroxybenzene/Acid Orange 24 (III) (C.I. 20170); sodium salt of 1-amino-2-(4′-nitrophenylazo)-7-phenylazo-8-hydroxynaphthalene-3,6-disulfonic acid/Acid Black 1 (III) (C.I. 20470); (4-((4-methylphenyl)sulfonyloxy)phenylazo)-2,2′-dimethyl-4-((2-hydroxy-5,8-disulfonato)naphthylazo)biphenyl/Acid Red 111 (III′) (C.I. 23266); Food Black 2 (III) (C.I. 27755); 1-(4′-sulfonatophenylazo)-4-((2″-hydroxy-3″-acetylamino-6″,8″-disulfonato)naphthylazo)-6-sulfonatonaphthalene (tetrasodium salt)/Food Black 1 (III) (C.I. 25440); Acid Blue 9 (VII) (C.I. 42090); Acid Violet 43 (V) (C.I. 60730); Acid Green 25 (V) (C.I. 61570); Sodium salt of 1-amino-4-cyclohexylamino-9,10-anthraquinone-2-sulfonic acid/Acid Blue 62 (V) (C.I. 62045); Acid Blue 78 (V) (C.I. 62105); Sodium salt of 4-hydroxy-3-((2-methoxyphenyl)azo)-1-naphthalenesulfonic acid/Acid Red 4 (III) (C.I. 14710); 2-piperidino 5-nitrobenzenesulfonic acid (VI′); 2-(4′-N,N-(2″-hydroxyethyl)amino-2′-nitro)anilineethanesulfonic acid (VI′); 4-β-hydroxyethylamino-3-nitrobenzenesulfonic acid (VI′); Acid Violet 49 (VII) (C.I. 42640); Acid Blue 7 (VII) (C.I. 42080); sodium salt of 1,2-dihydroxy-3-sulfoanthraquinone/Mordant Red 3 (V) (C.I. 58005); Sodium salt of 1-amino-9,10-dihydro-9,10-dioxo-4-(phenylamino)-2-anthracenesulfonic acid/Acid Blue 25 (V) (C.I. 62055); Sodium salt of 4-hydroxy-3-((2-methoxyphenyl)azo)-1-naphthalenesulfonic acid/Acid Red 4 (III) (C.I. 14710).

Most of these dyes are described in particular in the Colour Index published by The Society of Dyers and Colourists, P.O. Box 244, Perkin House, 82 Grattan Road, Bradford, Yorkshire, BD1 2JB, England.

The anionic dyes which are most particularly preferred are the dyes designated in the Colour Index under the code C.I. 58005 (monosodium salt of 1,2-dihydroxy-9,10-anthraquinone-3-sulfonic acid), C.I. 60730 (monosodium salt of 2-[(9,10-dihydro-4-hydroxy-9,10-dioxo-1-anthracenyl)amino]-5-methylbenzenesulfonic acid), C.I. 15510 (monosodium salt of 4-[(2-hydroxy-1-naphthalenyl)azo]benzenesulfonic acid), C.I. 15985 (disodium salt of 6-hydroxy-5-[(4-sulfophenyl)azo]-2-naphthalenesulfonic acid), C.I. 17200 (disodium salt of 5-amino-4-hydroxy-3-(phenylazo)-2,7-naphthalenedisulfonic acid), C.I. 20470 (disodium salt of 1-amino-2-(4′-nitrophenylazo)-7-phenylazo-8-hydroxy-3,6-naphthalenedisulfonic acid), C.I. 42090 (disodium salt of N-ethyl-N-[4-[[4-[ethyl[(3-sulfophenyl)methyl]amino]phenyl](2-sulfophenyl)methylene]-2,5-cyclohexadien-1-ylidene]-3-sulfobenzenemethanaminium hydroxide, internal salt), C.I. 61570 (disodium salt of 2,2′-[(9,10-dihydro-9,10-dioxo-1,4-anthracenediyl)diimino]bis[5-methyl]benzenesulfonic acid).

Use may also be made of the compounds corresponding to the mesomeric or tautomeric forms of the structures (III) to (X).

### Natural Dyes

The direct dyes may be chosen from natural direct dyes.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin, orceins, brazilin, brazilein, hematin and hematoxylin. Use may also be made of extracts or decoctions containing these natural dyes and notably henna-based poultices or extracts.

According to a preferred embodiment, the direct dyes are chosen from the triarylmethane direct dyes of formulae (IIa₁) and (IIa₂) below, and mixtures thereof:

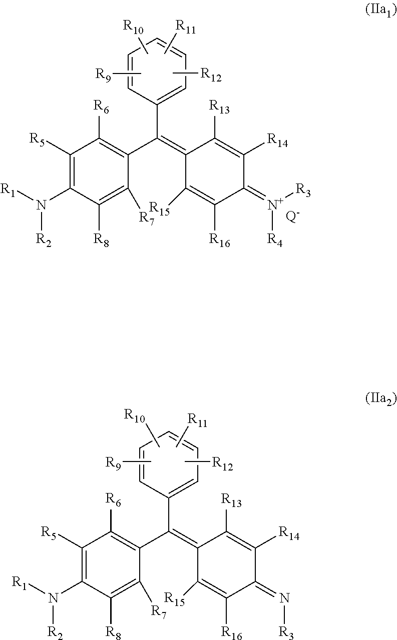

in which:
R₁, R₂, R₃ and R₄, which may be identical or different, represent a hydrogen atom or group from among: (C₁-C₆)alkyl which is optionally substituted, preferably with a hydroxyl group; aryl such as phenyl, aryl(C₁-C₄)alkyl such as benzyl, heteroaryl, heteroaryl(C₁-C₄)alkyl, or else two groups R₁ and R₂, and/or R₃ and R₄, borne by the same nitrogen atom, form, together with the nitrogen atom which bears them, an optionally substituted heterocycloalkyl group such as morpholino, piperazino or piperidino; preferably, R₁, R₂, R₃ and R₄, which may be identical or different, represent a hydrogen atom or a (C₁-C₄)alkyl group;
R₅, R₆, R₇, R₈, R₉, R₁₀, R₁₁, R₁₂, R₁₃, R₁₄, R₁₅, and R₁₆, which may be identical or different, represent a hydrogen or halogen atom, or a group chosen from i) hydroxyl, ii) thiol, iii) amino, iv) (di)(C₁-C₄)(alkyl)amino, v) (di)arylamino such as (di)phenylamino, vi) nitro, vii) acylamino (—NR—C(O)R′) in which the radical R is a hydrogen atom, a C₁-C₄ alkyl radical optionally bearing at least one hydroxyl group and the radical R′ is a C₁-C₂ alkyl radical; viii) carbamoyl ((R)₂N—C(O)—) in which the radicals R, which may be identical or different, represent a hydrogen atom or a C₁-C₄ alkyl radical optionally bearing at least one hydroxyl group; ix) carboxylic acid or ester, (—O—C(O)R′) or (—C(O)OR′), in which the radical R′ is a hydrogen atom or C₁-C₄ alkyl optionally bearing at least one hydroxyl group and the radical R′ is a C₁-C₂ alkyl radical; x) alkyl which is optionally substituted, notably with a hydroxyl group; xi) alkylsulfonylamino (R′SO₂—NR—) in which the radical R represents a hydrogen atom or a C₁-C₄ alkyl radical optionally bearing at least one hydroxyl group and the radical R′ represents a C₁-C₄ alkyl radical or a phenyl radical; xii) aminosulfonyl ((R)₂N—SO₂—) in which the radicals R, which may be identical or different, represent a hydrogen atom or a C₁-C₄ alkyl radical optionally bearing at least one hydroxyl group; xiii) (C₁-C₄)alkoxy; and xiv) (C₁-C₄)alkylthio;
or else two radicals borne by two contiguous carbon atoms R₅ and R₆ and/or R₇ and R₈, and/or R₉ and R₁₀ and/or R₁₁ and R₁₂ and/or R₁₃ and R₁₄ and/or R₁₅ and R₁₆ form, together with the carbon atoms which bear them, an aryl or heteroaryl, preferably benzo, 6-membered fused ring, said ring possibly also being optionally substituted, preferably an unsubstituted benzo ring;
Q⁻ represents an anionic counterion for achieving electrical neutrality, preferably chosen from halides such as chloride or bromide, and phosphate;

According to this preferred embodiment, the direct dye is preferably HC Blue 15.

According to a variant of the invention, the direct dye(s) are chosen from cationic, preferably endocyclic, direct dyes, more particularly of formula (IV-1) as defined previously, for instance Basic Red 51.

The direct dye(s) are preferably chosen from cationic direct dyes, more preferentially from the triarylmethane direct dyes of formulae (IIa₁) and (IIa₂) as defined previously, the direct dyes of formulae (II-1) and (IV-1) as defined previously, and mixtures thereof, even more preferentially from HC Blue 15, Basic Red 51, Basic Yellow 87 and mixtures thereof.

The direct dye(s) may be present in the composition in a total content ranging from 0.001% to 5% by weight, preferably from 0.01% to 3% by weight, more preferentially from 0.1% to 1% by weight, even more preferentially from 0.1% to 0.8% by weight, relative to the total weight of the composition.

According to a preferred embodiment, the composition comprises a total content of colouring agents of less than 0.1% by weight, preferably of less than 0.01% by weight, more preferentially of less than 0.001% by weight, relative to the total weight of the composition.

According to a more preferred embodiment, the composition is free of colouring agents.

### Additional Basifying Agents

The composition may also comprise one or more additional basifying agents other than the carbonates, bicarbonates and silicates as defined previously.

The additional basifying agent(s) may be mineral or organic. They may be chosen from i) aqueous ammonia, ii) alkanolamines such as monoethanolamine, diethanolamine, triethanolamine and derivatives thereof, iii) oxyethylenated and/or oxypropylenated ethylenediamines, iv) mineral or organic hydroxides, v) amino acids, preferably basic amino acids such as arginine, lysine, ornithine, citrulline and histidine, and vi) the compounds of formula (II) below:

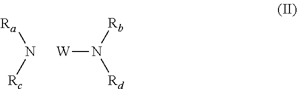

in which:
W is a divalent (C₁-C₈)alkylene group, preferably propylene optionally substituted notably with a hydroxyl group or a C₁-C₄ alkyl radical;
Rₐ, R_{b}, R_{c} and R_{d}, which may be identical or different, represent a hydrogen atom or a C₁-C₄ alkyl or C₁-C₄ (poly)hydroxyalkyl radical;

vii) and mixtures thereof.

The mineral or organic hydroxides are preferably chosen from i) alkali metal hydroxides such as sodium hydroxide or potassium hydroxide, ii) alkaline-earth metal hydroxides, iii) transition metal hydroxides, such as hydroxides of metals from groups III, IV, V and VI, iv) hydroxides of lanthanides or of actinides.

When they are present, the additional basifying agent(s) preferably represent from 0.001% to 20% by weight, more particularly from 0.005% to 16%, relative to the total weight of the composition.

According to a preferred embodiment, the composition according to the invention does not comprise any additional basifying agent chosen from aqueous ammonia and/or alkanolamines.

### Mineral Acidifying Agents

The composition may also comprise one or more mineral acidifying agents, for instance hydrochloric acid, (ortho)phosphoric acid, boric acid, nitric acid and sulfuric acid.

### pH of the Composition

The composition according to the invention preferably has a pH of less than or equal to 11, preferably less than or equal to 10.5, preferably less than or equal to 10.

The pH of the composition according to the invention may range from 8 to 11, preferably from 8 to 10.5, more preferentially from 8 to 10.

According to a particularly preferred embodiment, the pH of the composition according to the invention ranges from 8.3 to 10.

### Sequestrants

The composition according to the invention optionally contains one or more sequestrants. As examples of sequestrants that may be used in the present invention, mention may be made of N,N-dicarboxymethyl-L-glutamic acid and tetrasodium N,N-bis(carboxymethyl)-L-glutamate.

### Other Characteristics of the Composition

The composition preferably comprises water in a content ranging from 5% to 99% by weight, more preferentially ranging from 5% to 80% by weight, relative to the total weight of the composition.

The composition may also comprise at least one organic solvent.

The term “organic solvent” means an organic substance that is capable of dissolving another substance without chemically modifying it.

Examples or organic solvents that may be mentioned include lower C₂-C₄ alkanols, such as ethanol and isopropanol; polyols and polyol ethers, for instance 2-butoxyethanol, propylene glycol, propylene glycol monomethyl ether and diethylene glycol monoethyl ether and monomethyl ether, and also aromatic alcohols, for instance benzyl alcohol or phenoxyethanol, and mixtures thereof.

The organic solvents are present in proportions preferably ranging from 0.1% to 40% by weight, more preferentially from 1% to 30% by weight, even more preferentially from 1% to 25% by weight, relative to the total weight of the composition.

The composition according to the invention may be in liquid form, in the form of a serum, in thickened form, in particular a gel, a cream, a wax or a paste, or in foam form.

The composition according to the invention may also comprise one or more additional compounds chosen from nonionic, anionic, cationic or amphoteric surfactants, cationic, anionic, nonionic or zwitterionic, associative or non-associative thickening polymers of natural or synthetic origin, silicones in the form of oil, gums or resins or non-silicone plant, mineral or synthetic oils, UV-screening agents, pigments, fillers, such as nacres and metal oxides such as titanium dioxides, clays, fragrances, peptizers, vitamins and preserving agents.

### Process for Lightening Keratin Fibres

According to a second aspect, a subject of the present invention is a process for lightening keratin fibres, comprising the application to the keratin fibres of a composition comprising ingredients i) to iv) and iv′) as defined previously.

### Process for the Simultaneous Bleaching and Dyeing of Keratin Fibres

According to a third aspect, a subject of the present invention is a process for the simultaneous bleaching and dyeing of keratin fibres, comprising the application to the keratin fibres of a composition comprising ingredients i) to iv) and iv′) as defined previously and v) one or more colouring agents, preferably chosen from direct dyes, oxidation dyes and mixtures thereof.

### Additional Characteristics of the Process for the Simultaneous Lightening or Bleaching and Dyeing of Keratin Fibres

In particular, the composition is applied to wet or dry keratin fibres.

Preferably, the keratin fibres are dark keratin fibres.

The term “dark keratin fibres” means keratin fibres with a tone depth of less than or equal to 6 (dark blond) and preferably less than or equal to 4 (chestnut-brown).

The composition may advantageously be applied to the keratin fibres in an amount ranging from 0.1 g to 20 g of composition per gram of keratin fibres.

The composition is left to stand on the fibres for a period generally from 1 minute to 1 hour, preferably from 5 minutes to 60 minutes.

By way of example, the composition may be left to stand on the fibres for a period of 50 minutes.

The composition may be left to stand on the fibres under an occlusive system. A non-limiting example of an occlusive system that may be mentioned is an occlusive system of envelope type made of aluminium or plastic film or a hair cap with or without holes.

The temperature during the lightening process is conventionally between ambient temperature (between 15° C. and 25° C.) and 80° C., and preferably between ambient temperature and 60° C.

By way of example, the temperature during the lightening process is 33° C.

After the treatment, the keratin fibres are optionally rinsed with water, optionally washed with a shampoo and then rinsed with water, before being dried or left to dry naturally.

The drying may be performed using absorbent paper, a hairdryer or a styling hood, or any other drying means.

The composition used in the process according to the present invention is preferably prepared by mixing at least two compositions. Preferably, the mixing of said at least two compositions is performed extemporaneously, before application of the composition to the keratin fibres.

According to a preferred embodiment, the composition used in the process according to the invention is derived from the mixing:
of a composition (A) comprising:
one or more chemical oxidizing agents chosen from hydrogen peroxide, hydrogen peroxide-generating systems other than peroxygenated salts, and mixtures thereof as defined previously, preferably hydrogen peroxide; and
optionally one or more alcohols iv) as defined previously, and/or one or more compounds iv′) as defined previously; and
a composition (B) comprising:
one or more compounds chosen from carbonates, carbonate-generating systems, bicarbonates, bicarbonate-generating systems and mixtures thereof as defined previously, preferably chosen from carbonates and bicarbonates, and mixtures thereof as defined previously; and
one or more silicates as defined previously; and
optionally one or more colouring agents, preferably chosen from direct dyes and oxidation dyes, and mixtures thereof; and
optionally one or more alcohols iv) as defined previously, and/or one or more compounds iv′) as defined previously; and
optionally a composition (C) comprising:
one or more colouring agents, preferably chosen from direct dyes and oxidation dyes, and/or mixtures thereof; and
optionally one or more alcohols iv) as defined previously, and/or one or more compounds iv′) as defined previously;

it being understood that:
at least one of the compositions (A) or (B) or at least one of the compositions (A) or (B) or (C), if composition (C) is present, comprises one or more alcohols iv) as defined previously; and
at least one of the compositions (A) or (B) or at least one of the compositions (A) or (B) or (C), if composition (C) is present, comprises one or more compounds iv′) as defined previously.

The alcohols iv) are preferably present in composition (B) or in at least one of the compositions (B) or (C), if composition (C) is present.

When they are present, the organic acids a) are preferably present in composition (A) or in at least one of the compositions (A) or (C), preferably in composition (C), if composition (C) is present.

When they are present, the amphoteric or zwitterionic compounds b) are preferably present in composition (A) or in at least one of the compositions (A) or (C), if composition (C) is present.

When they are present, the compounds c) are preferably present in composition (B) or in at least one of the compositions (B) or (C), if composition (C) is present.

When they are present, the organic amines d) are preferably present in composition (B) or in at least one of the compositions (B) or (C), if composition (C) is present.

Preferably, when the composition according to the invention is derived from the mixing of compositions (A), (B) and (C) as defined previously, composition (B) does not comprise any colouring agent(s) chosen from direct dyes, oxidation dyes and mixtures thereof.

Preferably, at least one of the compositions (A) or (B) or at least one of the three compositions (A) or (B) or (C) is aqueous. More preferentially, composition (A) is aqueous.

According to a particular embodiment, composition (B) is anhydrous.

According to a particular embodiment, composition (A) is aqueous, composition (B) is anhydrous and composition (C), when it is present, is aqueous.

The term “aqueous composition” means a composition comprising at least 2% by weight of water, preferably at least 5% by weight of water, more preferentially at least 10% by weight of water and even more advantageously more than 20% by weight of water.

The term “anhydrous composition” refers to a composition containing less than 2% by weight of water, preferably less than 0.5% by weight of water, and more preferentially free of water. Where appropriate, such small amounts of water may notably be introduced by ingredients of the composition that may contain residual amounts thereof.

The composition used in the process according to the present invention makes it possible to obtain a lightening of keratin fibres characterized by a b* value that is lower, preferably by 10% and more preferentially by 15%, than the b* value measured at the same intensity level L* on keratin fibres lightened using a composition comprising one or more persulfates, the b* and L* values being measured in the CIE L*a*b* system.

Preferably, the values of b* and L* are measured according to the colour evaluation method described in the examples.

### Uses

According to a fourth aspect, a subject of the present invention is the use of a composition comprising ingredients i) to iv) and iv′) as defined previously for lightening keratin fibres, preferably for lightening keratin fibres while at the same time de-yellowing them.

According to a fifth aspect, a subject of the present invention is the use of a composition comprising ingredients i) to iv) and iv′) as defined previously and v) one or more colouring agents, preferably chosen from direct dyes, oxidation dyes and mixtures thereof, for the simultaneous bleaching and dyeing of keratin fibres.

### Multi-Compartment Device (Kit)

According to a sixth aspect, a subject of the present invention is a device containing several separate compartments (kit), comprising:
a first compartment containing a composition (A) comprising:
one or more chemical oxidizing agents chosen from hydrogen peroxide, hydrogen peroxide-generating systems other than peroxygenated salts, and mixtures thereof, preferably hydrogen peroxide; and
optionally one or more alcohols iv) and/or one or more compounds iv′) as defined previously; and
a second compartment containing a composition (B) comprising:
one or more compounds chosen from carbonates, carbonate-generating systems, bicarbonates, bicarbonate-generating systems and mixtures thereof, preferably chosen from carbonates and bicarbonates, and mixtures thereof; and
one or more silicates; and
optionally one or more colouring agents, preferably chosen from direct dyes and oxidation dyes, and mixtures thereof; and
optionally one or more alcohols iv) and/or one or more compounds iv′) as defined previously; and
optionally a third compartment containing a composition (C) comprising:
one or more colouring agents, preferably chosen from direct dyes and oxidation dyes, and mixtures thereof; and
optionally one or more alcohols iv) and/or one or more compounds iv′) as defined previously;

it being understood that:
at least one of the compositions (A) or (B) or at least one of the compositions (A) or (B) or (C), if composition (C) is present, comprises one or more alcohols iv); and
at least one of the compositions (A) or (B) or at least one of the compositions (A) or (B) or (C), if composition (C) is present, comprises one or more compounds iv′) as defined previously.

### EXAMPLES

The examples that follow will allow the invention to be understood more clearly, without, however, being limiting in nature. In the examples which follow, unless otherwise indicated, all the amounts are shown as mass percentages relative to the total weight of the composition.

### Method for Evaluating the Colour

In these examples, the colour of the locks was evaluated in the CIE L* a* b* system, using a colorimeter which is a Minolta CM3610A Spectrophotometer (illuminant 065).

In this L*a*b* system, L* represents the intensity of the colour, a* indicates the shade of the colour on the green/red colour axis and b* indicates the shade of the colour on the blue/yellow colour axis. The higher the value of L*, the lighter the colour. The higher the value of a*, the redder the colour and the higher the value of b*, the yellower the colour.

### Example 1 (Comparative)

Compositions C1 to C12 below were prepared and then applied according to the application protocol described below:

TABLE 1

| Ingredients | C1 | C2 | C3 | C4 | C5 | C6 |
|---|---|---|---|---|---|---|
| Oxidizing cream | 70.0 (i.e. | 70.0 (i.e. | 70.0 (i.e. | 70.0 (i.e. | 70.0 (i.e. | 70.0 (i.e. |
| Blond Studio 40 Vol. | 8.4 of | 8.4 of | 8.4 of | 8.4 of | 8.4 of | 8.4 of |
| L'Oréal Professionnel | H₂O₂) | H₂O₂) | H₂O₂) | H₂O₂) | H₂O₂) | H₂O₂) |
| (12% of H₂O₂) | | | | | | |
| Potassium persulfate | 15.0 | 12.5 | 10.0 | 8.0 | 6.0 | 4.0 |
| Sodium silicate | 10.0 | 10.0 | 10.0 | 8.0 | 8.0 | 8.0 |
| (CAS: 1344-09-8) | | | | | | |
| Water | qs 100 | qs 100 | qs 100 | qs 100 | qs 100 | qs 100 |

TABLE 2

| Ingredients | C7 | C8 | C9 | C10 | C11 | C12 |
|---|---|---|---|---|---|---|
| Oxidizing cream | 70.0 (i.e. | 70.0 (i.e. | 70.0 (i.e. | 70.0 (i.e. | 70.0 (i.e. | 70.0 (i.e. |
| Blond Studio 40 Vol. | 8.4 of | 8.4 of | 8.4 of | 8.4 of | 8.4 of | 8.4 of |
| L'Oréal Professionnel | H₂O₂) | H₂O₂) | H₂O₂) | H₂O₂) | H₂O₂) | H₂O₂) |
| (12% of H₂O₂) | | | | | | |
| Potassium persulfate | 8.0 | 6.0 | 4.0 | 8.0 | 6.0 | 4.0 |
| Sodium silicate | 6.0 | 6.0 | 6.0 | 4.0 | 4.0 | 4.0 |
| (CAS: 1344-09-8) | | | | | | |
| Water | qs 100 | qs 100 | qs 100 | qs 100 | qs 100 | qs 100 |

### Application Protocol

10 g of each of the compositions C1 to C12 are applied to twelve 1-g locks of Caucasian HT4 dark hair on a hot plate maintained at a temperature of 33° C. The whole is covered with a cellophane film for 50 minutes.

The locks are then rinsed, washed with a standard shampoo, rinsed again and then dried.

### Colorimetric Measurements

The results of the colorimetric measurements are summarized in the following table:

TABLE 3

| Composition | L* | b* |
|---|---|---|
| C1 | 57.37 | 30.95 |
| C2 | 56.34 | 31.74 |
| C3 | 54.6 | 30.25 |
| C4 | 54.05 | 30.19 |
| C5 | 50.62 | 28.88 |
| C6 | 48.51 | 29.48 |
| C7 | 49.91 | 29.48 |
| C8 | 47.53 | 27.87 |
| C9 | 46.64 | 26.82 |
| C10 | 47.01 | 27.04 |
| C11 | 45.33 | 26.08 |
| C12 | 41.44 | 23.77 |

The results show that comparative compositions comprising a persulfate make it possible to obtain a good level of lightening characterized by relatively high L* values, but the colour shades obtained have a pronounced yellow component characterized by high b* values.

### Example 2

Compositions C13 to C21 below were prepared and then applied according to the application protocol described below:

TABLE 4

| Ingredients | C13 | C14 | C15 | C16 | C17 |
|---|---|---|---|---|---|
| Oxidizing cream | 60.0 (i.e. | 60.0 (i.e. | 60.0 (i.e. | 60.0 (i.e. | 60.0 (i.e. |
| Blond Studio 30 | 5.4 of | 5.4 of | 5.4 of | 5.4 of | 5.4 of |
| Vol. L'Oréal | H₂O₂) | H₂O₂) | H₂O₂) | H₂O₂) | H₂O₂) |
| Professionnel | | | | | |
| (9% of H₂O₂) | | | | | |
| Sodium silicate | 13.0 | 13.0 | 13.0 | 13.0 | 13.0 |
| (CAS: 1344-09-8) | | | | | |
| Sodium metasilicate | 4.5 | 4.5 | 4.5 | 4.5 | 4.5 |
| (CAS: 6834-92-0) | | | | | |
| Ammonium | 13.33 | 13.33 | 13.33 | 13.33 | 13.33 |
| bicarbonate | | | | | |
| Glyceryl stearate | 4.585 | — | — | — | — |
| citrate | | | | | |
| (Compound 31) | | | | | |
| Pentylene glycol | 4.585 | — | — | — | — |
| (Compound 18a) | | | | | |
| Betaine | — | 3.95 | 4.585 | — | — |
| (Compound 15b) | | | | | |
| Citric acid | — | 1.54 | — | — | — |
| (Compound 26) | | | | | |
| Glycerol | — | 3.68 | 4.585 | — | — |
| (Compound 12a) | | | | | |
| Behenyl alcohol | — | — | — | 4.585 | — |
| (Compound 16a) | | | | | |
| Polyglyceryl-2 | — | — | — | 4.585 | — |
| dipolyhydroxystearate | | | | | |
| (Compound 58c) | | | | | |
| 2-Phenylethanol | — | — | — | — | 1.0 |
| (Compound 26a) | | | | | |
| Sorbitan oleate | — | — | — | — | 0.184 |
| (Compound 4c) | | | | | |
| Water | — | — | — | — | qs 100 |

TABLE 5

| Ingredients | C18 | C19 | C20 | C21 |
|---|---|---|---|---|
| Oxidizing cream | 60.0 (i.e. | 60.0 (i.e. | 60.0 (i.e. | 60.0 (i.e. |
| Blond Studio 30 | 5.4 of | 5.4 of | 5.4 of | 5.4 of |
| Vol. L'Oréal | H₂O₂) | H₂O₂) | H₂O₂) | H₂O₂) |
| Professionnel (9% | | | | |
| of H₂O₂) | | | | |
| Sodium silicate | 13.0 | 13.0 | 13.0 | 13.0 |
| (CAS: 1344-09-8) | | | | |
| Sodium metasilicate | 4.5 | 4.5 | 4.5 | 4.5 |
| (CAS: 6834-92-0) | | | | |
| Ammonium | 13.33 | 13.33 | 13.33 | 13.33 |
| bicarbonate | | | | |
| Oleyl alcohol | 4.585 | — | — | — |
| (Compound 1a) | | | | |
| Stearamidopropyl | 4.585 | — | — | — |
| dimethylamine | | | | |
| (Compound 30d) | | | | |
| Dicaprylyl ether | — | 4.585 | — | — |
| (Compound 2e) | | | | |
| Glycerol | — | 4.585 | 4.585 | 4.585 |
| (Compound 12a) | | | | |
| L-lysine | — | — | 4.585 | — |
| (Compound 4b) | | | | |
| Chitosan | — | — | — | 4.585 |
| MW: 20 kDa | | | | |

### Application Protocol

10 g of each of the compositions C13 to C21 are applied to seven 1-g locks of Caucasian HT4 dark hair on a hot plate maintained at a temperature of 33° C. The whole is covered with a cellophane film for 50 minutes.

The locks are then rinsed, washed with a standard shampoo, rinsed again and then dried.

### Colorimetric Measurements

The results of the colorimetric measurements are summarized in the following table:

TABLE 6

| Composition | L* | b* |
|---|---|---|
| C13 | 48.32 | 23.79 |
| C14 | 50.37 | 23.98 |
| C15 | 47.1 | 22.39 |
| C16 | 50.14 | 23.2 |
| C17 | 46.71 | 24.77 |
| C18 | 50.82 | 23.16 |
| C19 | 47.69 | 23.19 |
| C20 | 47.57 | 23.05 |
| C21 | 44.30 | 22.69 |

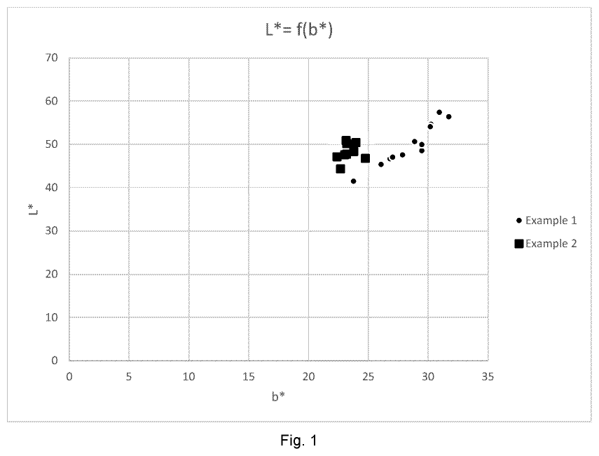

The results show that compositions according to the invention make it possible to obtain a good level of lightening characterized by relatively high L* values. Furthermore, the colour shades obtained are characterized by lower b* values at an equivalent L* intensity level for the compositions according to the present invention than for the comparative persulfate-based compositions of Example 1, as illustrated in FIG. 1.

1-30. (canceled)

31. A composition comprising:
i) one or more chemical oxidizing agents chosen from hydrogen peroxide, hydrogen peroxide-generating systems other than peroxygenated salts, or mixtures thereof;
ii) one or more compounds chosen from carbonates, carbonate-generating systems, bicarbonates, bicarbonate-generating systems, or mixtures of two or more thereof;
iii) one or more silicates;
iv) one or more alcohols;
v) one or more compounds different from the one or more alcohols iv), chosen from:
a) organic acids;
b) amphoteric or zwitterionic compounds chosen from phospholipids, amino acids, amphoteric or zwitterionic surfactants, or mixtures of two or more thereof;
c) esters, amides, imines, or mixtures of two or more thereof;
d) organic amines;
e) compounds comprising a saturated or unsaturated, linear or branched hydrocarbon-based chain comprising from 1 to 30 carbon atoms; or
mixtures of two or more thereof;
wherein the total amount of silicate(s) iii) ranges from 2% to 35% by weight relative to the total weight of the composition; and
wherein the composition comprises a total amount of persulfates of less than 5% by weight, relative to the total weight of the composition.

32. The composition of claim 31, wherein the chemical oxidizing agent i) is hydrogen peroxide.

33. The composition of claim 31, wherein the total amount of chemical oxidizing agent(s) i) chosen from hydrogen peroxide, hydrogen peroxide-generating systems other than peroxygenated salts, or mixtures thereof ranges from 1% to 12% by weight, relative to the total weight of the composition.

34. The composition of claim 31, wherein the total amount of ii) one or more compounds chosen from carbonates, carbonate-generating systems, bicarbonates, bicarbonate-generating systems, or mixtures of two or more thereof ranges from 0.01% to 20% by weight, relative to the total weight of the composition.

35. The composition of claim 31, wherein the one or more compounds ii) is chosen from carbonates, carbonate-generating systems, or mixtures thereof.

36. The composition of claim 35, wherein total amount of carbonate(s) and/or carbonate-generating system(s), or mixtures thereof ranges from 0.01% to 20% by weight, relative to the total weight of the composition.

37. The composition of claim 31, wherein the carbonate(s) is chosen from:
alkali metal carbonates;
alkaline-earth metal carbonates;
lanthanide carbonates;
transition metal carbonates;
bismuth carbonate;
cadmium carbonate;
thallium carbonate;
zinc carbonate;
compounds of formula (N⁺R¹R²R³R⁴)₂CO₃²⁻ wherein R¹, R², R³ and R⁴ are independently chosen from a hydrogen atom or a (C₁-C₄)alkyl group optionally substituted with a hydroxyl group;
guanidine carbonate; or
mixtures of two or more thereof.

38. The composition of claim 31, wherein the one or more compounds ii) is chosen from bicarbonates, bicarbonate-generating systems, or mixtures thereof.

39. The composition of claim 31, wherein the total amount of bicarbonate(s) and/or the bicarbonate-generating system(s) ranges from 0.01% to 20% by weight, relative to the total weight of the composition.

40. The composition of claim 31, wherein the bicarbonate(s) is chosen from:
alkali metal bicarbonates;
alkaline-earth metal bicarbonates;
compounds of formula N⁺R¹R²R³R⁴HCO₃⁻ wherein R¹, R², R³ and R⁴ are independently chosen from a hydrogen atom or a (C₁-C₄)alkyl group optionally substituted with a hydroxyl group;
aminoguanidine bicarbonate; or
mixtures of two or more thereof.

41. The composition of claim 31, wherein the one or more silicates iii) is chosen from alkali metal silicates, alkaline-earth metal silicates, aluminium silicates, trimethylammonium silicates, or mixtures of two or more thereof.

42. The composition of claim 31, wherein the total amount of silicate(s) iii) ranges from 3% to 35% by weight, relative to the total weight of the composition.

43. The composition of claim 31, wherein the weight ratio of the total amount of bicarbonate(s) and/or bicarbonate-generating system(s) to the total amount of silicate(s) iii) ranges from 1:50 to 7.5:1.

44. The composition of claim 31, wherein the weight ratio of the total amount of bicarbonate(s) and/or bicarbonate-generating system(s) to the total amount of chemical oxidizing agent(s) i) ranges from 11:100 to 5:1.

45. The composition of claim 31, wherein the weight ratio of the total amount of carbonate(s) and/or carbonate-generating system(s) to the total amount of bicarbonate(s) and/or bicarbonate-generating system(s) ranges from 3:50 to 20:1.

46. The composition of claim 31, wherein the one or more alcohols iv) is chosen from the compounds of formula (Ia), salts thereof, optical isomers thereof, geometrical isomers thereof, tautomers thereof, solvates thereof, or mixtures of two or more thereof: 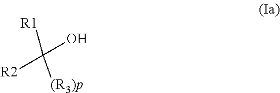
wherein in formula (Ia):
R¹ and R² are independently chosen from:
a hydrogen atom; or
a linear or branched, saturated or unsaturated hydrocarbon-based group comprising from 1 to 30 carbon atoms, optionally substituted with one or more non-adjacent oxygen atoms and/or optionally substituted with one or more identical or different groups chosen from: hydroxyl (—OH) or (poly)(hydroxy)alkyl; or
a (hetero)cyclic group optionally substituted with one or more identical or different groups chosen from: —OR″ or hydroxyl (—OH); or
R¹ and R² form together with the carbon atom which bears them, a (hetero)cyclic group optionally substituted with one or more identical or different groups chosen from:
a hydroxyl group (—OH);
a (poly)(hydroxy)alkyl group;
a group —OR″;
an alkyl group optionally substituted with one or more carbonyl groups (CO) and/or optionally substituted with a (hetero)cyclic group, the (hetero)cyclic group being optionally substituted with one or more identical or different groups chosen from: hydroxyl (—OH) or (poly)(hydroxy)alkyl;
a group optionally substituted with a (hetero)cyclic group, the (hetero)cyclic group itself being optionally substituted with one or more identical or different groups chosen from: hydroxyl (—OH) or (poly)(hydroxy)alkyl;
R³ represents:
a linear or branched, saturated or unsaturated or aromatic hydrocarbon-based group comprising from 1 to 30 carbon atoms, optionally substituted with one or more non-adjacent oxygen atoms or one or more carbonyl (CO) groups and/or optionally substituted with one or more identical or different groups chosen from: hydroxyl (—OH) or (poly)(hydroxy)alkyl or —CHO or (hetero)cyclic, the (hetero)cyclic group itself being optionally substituted with one or more identical or different groups chosen from: hydroxyl (—OH) or (poly)(hydroxy)alkyl or —OR″ or —CHO; or
a (hetero)cyclic group optionally substituted with one or more identical or different groups chosen from: —OR″ or (poly)(hydroxy)alkyl or —CHO;
p=0 or 1;
R″ represents:
a linear or branched, saturated or unsaturated hydrocarbon-based group comprising from 1 to 30 carbon atoms, optionally substituted with one or more hydroxyl groups (—OH); or
a (hetero)cyclic group optionally substituted with one or more identical or different groups chosen from: hydroxyl (—OH) or (poly)(hydroxy)alkyl.

47. The composition of claim 31,
wherein the organic acid a) is chosen from compounds of formulae (I) or (II), salts thereof, optical isomers thereof, geometrical isomers thereof, tautomers thereof, solvates thereof, or mixtures of two or more thereof: 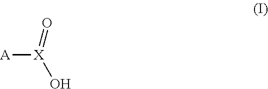
wherein in formula (I):
X represents a carbon atom, S═O, or P—OH;
A represents a group —OR⁴ or a group —CR¹(R²)p(R³), with the proviso that if X represents P—OH, then A represents a group —OR⁴;
p=0 or 1;
R¹ represents a hydrogen atom, a hydroxyl group (—OH), a group —NR^{a}—R^{c}, or a group —NR^{a}—CO—R^{c};
R² represents a hydrogen atom, a hydroxyl group (—OH), a group —X(O)OH, with X as defined previously;
R³ represents a hydrogen atom, a hydroxyl group (—OH), or a (hetero)cyclic group optionally substituted with one or more identical or different groups chosen from (C₁-C₆)alkyl substituted; or
R¹ and R³ form together with the carbon atom which bears them, a (hetero)cyclic group optionally substituted with one or more identical or different groups chosen from (C₁-C₆)alkyl, alkylcarbonyl —(CO)—R^{c}, alkylcarbonyloxy —O—CO—R^{c}, alkyloxycarbonyl —CO—O—R^{c}, —OH, (C₁-C₆)alkoxy, or —X(O)OH with X as defined previously; or
R¹ and R³ form, together with the carbon atom bearing them, a carbonyl group —CO—;
R⁴ represents a hydrogen atom, r an alkyl group, an alkenyl group, or a (hetero)cyclic group, said alkyl, alkenyl, or (hetero)cyclic group being optionally substituted with one or more identical or different groups chosen from a group —X(O)OH with X as defined previously;
R^{a} represents a hydrogen atom or a (C₁-C₄)alkyl group optionally substituted with one or more groups —X(O)OH with X as defined previously;
R^{c} represents an alkyl group, an alkenyl group, an alkylamino group, or an alkylamido group, said alkyl, alkenyl, alkylamino, or alkylamido group being:
optionally substituted with one or more identical or different groups chosen from a group —X(O)OH with X as defined previously, a hydroxyl group (—OH) or a (hetero)cyclic group; and/or
optionally substituted with one or more heteroatoms or groups chosen from —O—, —S—, —CO—, —O—CO—, —O—CO—, —NR^{a}—(CO)—, or —(CO)—NR^{a}—; 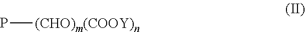
wherein in formula (II):
P represents a polysaccharide chain of monosaccharides comprising 5 or more carbon atoms;
Y is chosen from a hydrogen atom, ions derived from an alkali metal or from an alkaline-earth metal, ammonia, organic amines, or basic amino acids;
m+n is greater than or equal to 1;
m is such that the degree of substitution of the polysaccharide with one or more aldehyde groups (DS(CHO)) ranges from 0.001 to 2;
n is such that the degree of substitution of the polysaccharide with one or more carboxylic groups (DS(COOX)) ranges from 0 to 2;
and/or
wherein the amphoteric or zwitterionic compound b) is chosen from compounds of formulae 1b to 28b, salts thereof, optical isomers thereof, geometrical isomers thereof, tautomers thereof, solvates thereof, or mixtures of two or more thereof: 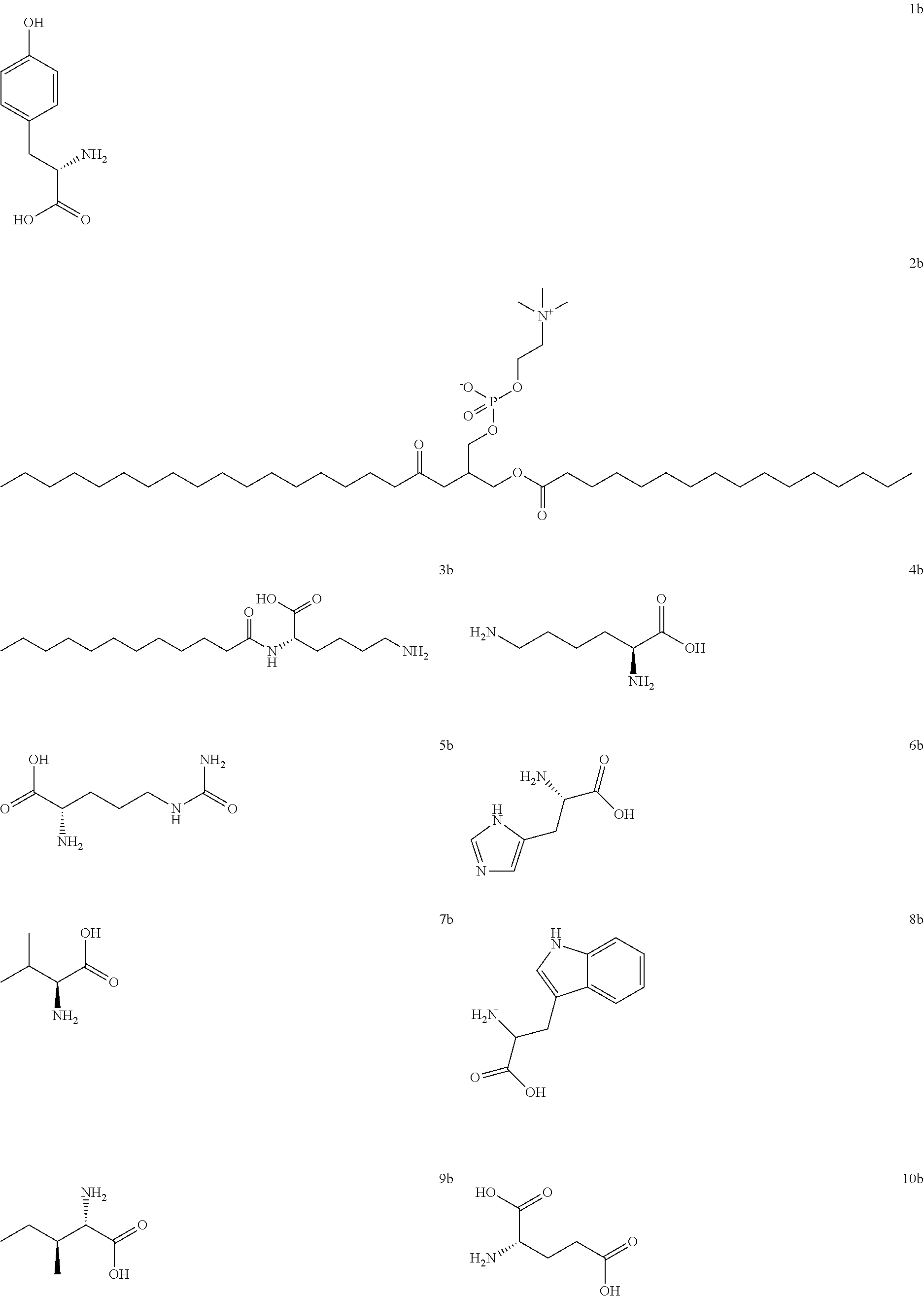 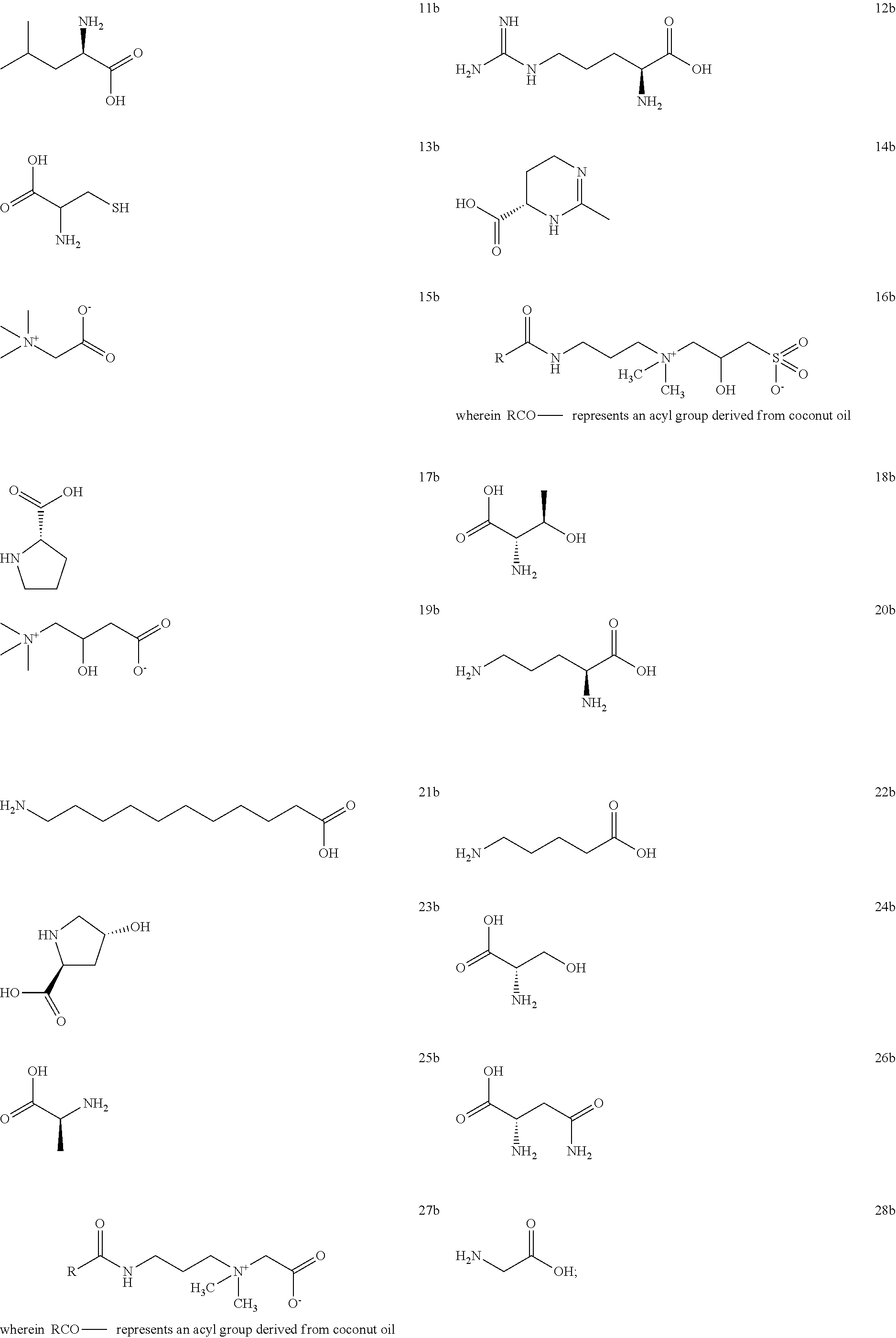
and/or
wherein the compound c) is chosen from compounds of formula (Ic), salts thereof, optical isomers thereof, geometrical isomers thereof, tautomers thereof, solvates thereof, or mixtures thereof: 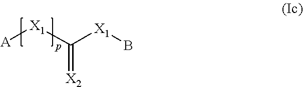
wherein in formula (Ic):
A represents a linear or branched, saturated or unsaturated hydrocarbon-based group comprising from 1 to 30 carbon atoms, optionally substituted with one or more non-adjacent oxygen atoms and/or with one or more identical or different groups chosen from: —CO—, —OCO—, —COO—, and/or optionally substituted with one or more identical or different groups chosen from a hydroxyl group (—OH), a group —COOAk1, or a (hetero)cyclic group optionally substituted with one or more hydroxyl groups (—OH);
X₁ independently represents an oxygen atom or a group —NR—;
X₂ represents an oxygen atom or a group —NR—;
p=0 or 1;
B represents a saturated or unsaturated, linear or branched or cyclic hydrocarbon-based group comprising from 1 to 30 carbon atoms, optionally substituted with one or more identical or different groups chosen from:
a (hetero)cyclic group optionally substituted with one or more identical or different groups chosen from: hydroxyl (—OH), —OR″, —COOA′, —OCOA′, or (poly)(hydroxy)alkyl;
a group —OR′;
a group —COOA′;
a group —OCOA′;
and/or optionally substituted with one or more non-adjacent oxygen atoms and/or with one or more identical or different groups chosen from —OCO— or —COO—;
A′ represents a linear or branched, saturated or unsaturated hydrocarbon-based group comprising from 1 to 30 carbon atoms, optionally substituted with one or more non-adjacent oxygen atoms and/or with one or more identical or different groups chosen from: —CO—, —OCO—, —COO—, and/or optionally substituted with one or more identical or different groups chosen from hydroxyl (—OH), or —COOAk1;
R represents a hydrogen atom or an alkyl group;
R′ represents a hydrogen atom or an alkyl group optionally substituted with one or more identical or different groups chosen from hydroxyl (—OH) or —OCOA′;
R″ represents:
an alkyl group optionally substituted with one or more hydroxyl groups (—OH); or
a (hetero)cyclic group optionally substituted with one or more identical or different groups chosen from hydroxyl (—OH), or (poly)(hydroxy)alkyl;
Ak1 represents an alkyl group optionally substituted with —COOAk2;
Ak2 represents an alkyl group;
wherein R and B together with the nitrogen atom bearing them may form a heterocycle;
wherein A and B together may form a heterocycle;
with the proviso that if X₁ represents a group —NR—, then A and B may independently represent a hydrogen atom;
and/or
wherein the organic amine d) is chosen from compounds of formulae 1d to 37d, chitosans thereof, salts thereof, optical isomers thereof, geometrical isomers thereof, tautomers thereof, solvates thereof, or mixtures of two or more thereof: 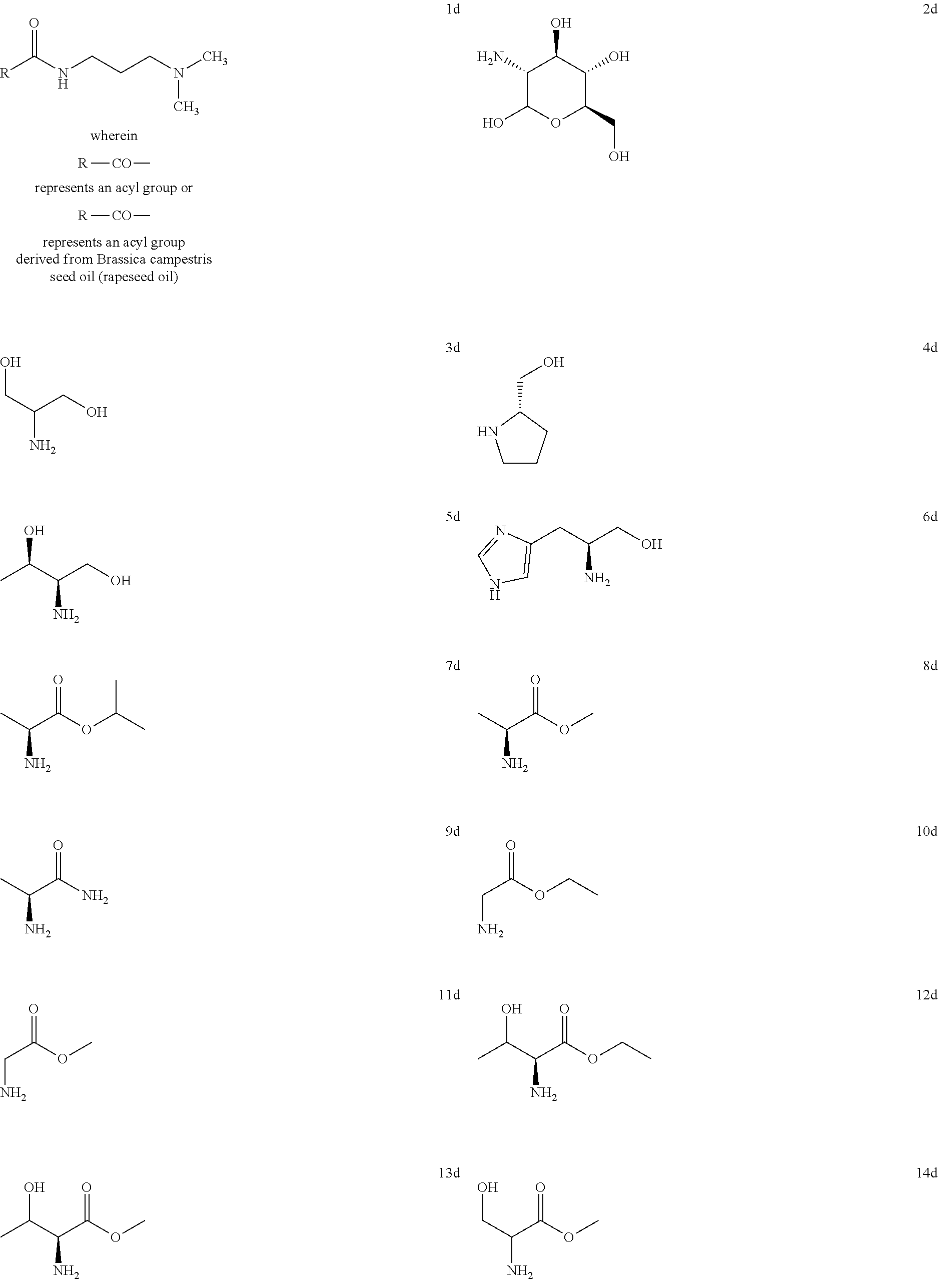 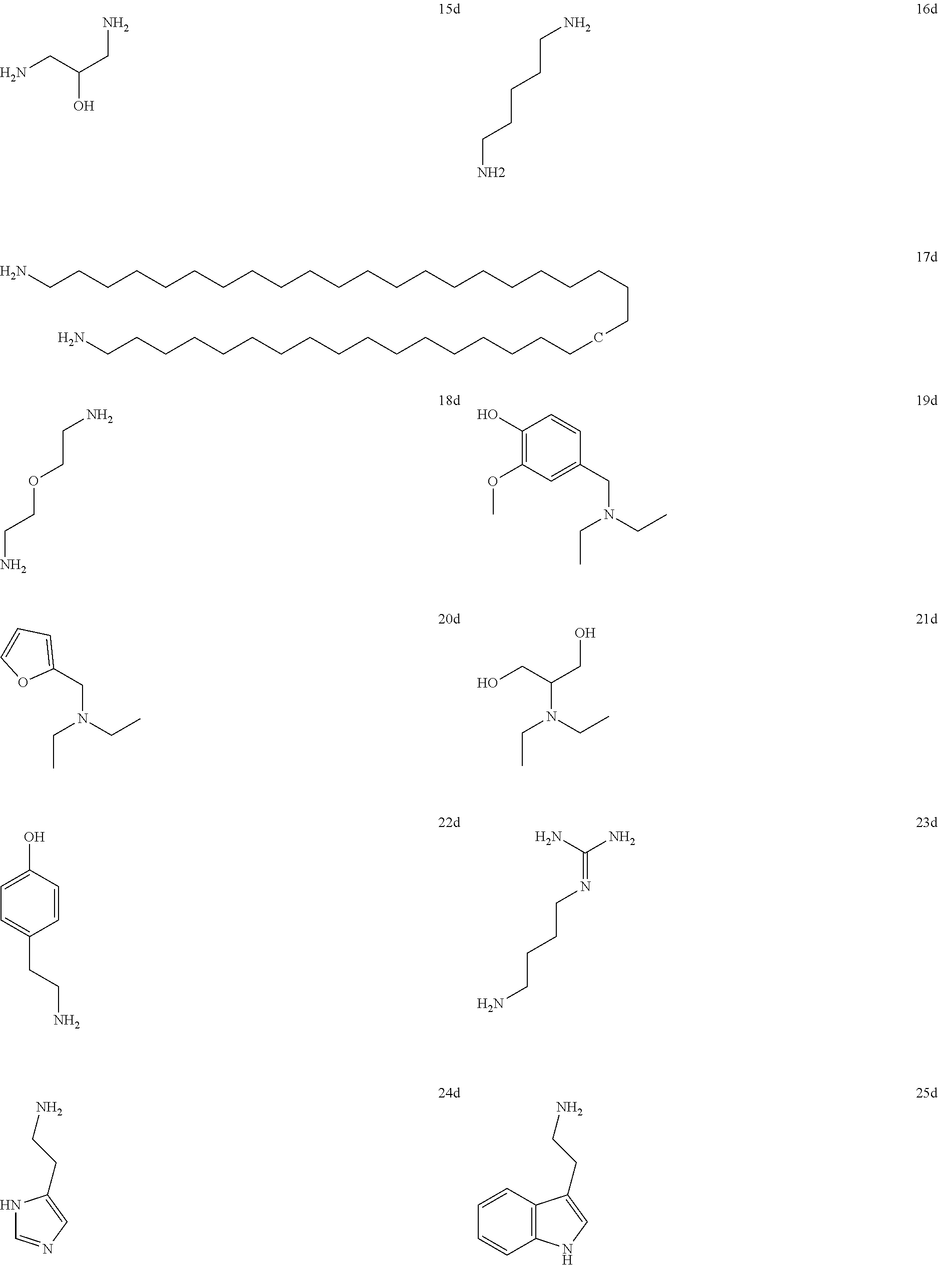 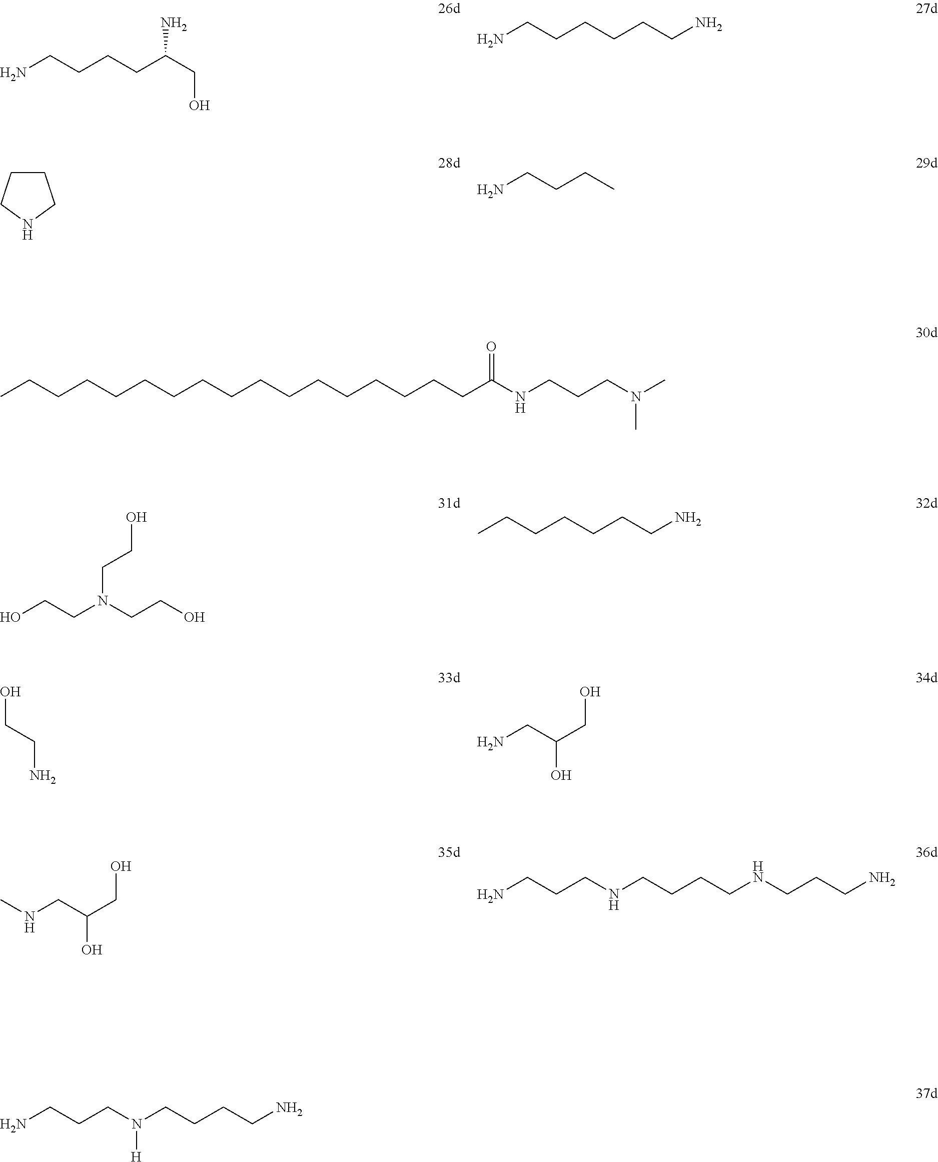
and/or
wherein the compound e) is chosen from compounds of formulae 1e to 18e, optical isomers thereof, geometrical isomers thereof, tautomers thereof, solvates thereof, or mixtures of two or more thereof:

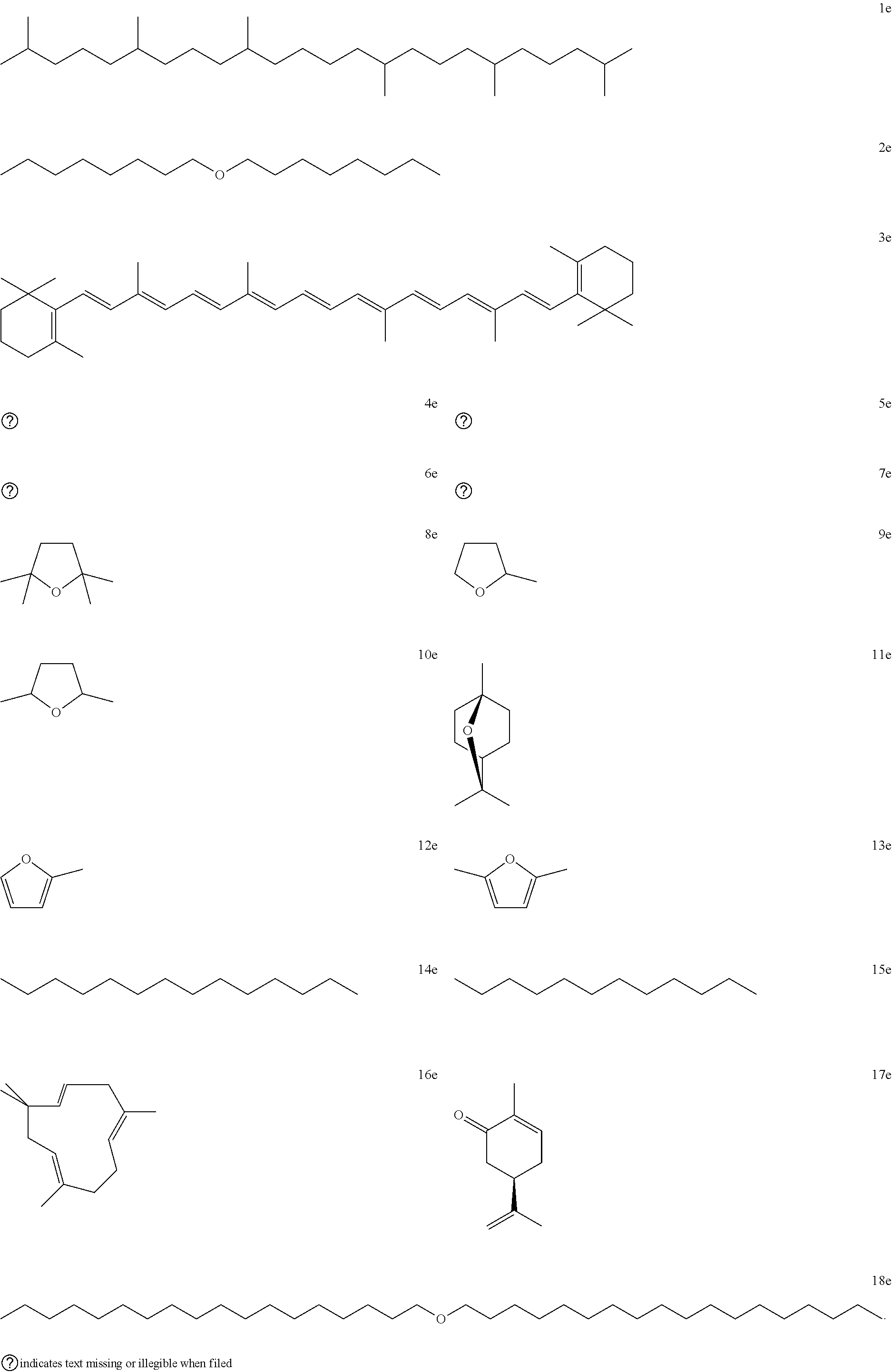

48. The composition of claim 31,
wherein the total amount of alcohol(s) iv) ranges from 1% to 30% by weight, relative to the total weight of the composition; and/or
wherein the total amount of compound(s) v) ranges from 0.1% to 30% by weight, relative to the total weight of the composition.

49. A method for simultaneously bleaching and dyeing keratin fibers, comprising:
(1) mixing a composition (A) with a composition (B), and optionally a composition (C): wherein composition (A) comprises:
one or more chemical oxidizing agents i) chosen from hydrogen peroxide, hydrogen peroxide-generating systems other than peroxygenated salts, or mixtures thereof; and
optionally one or more alcohols iv) and/or one or more compounds v) different from the one or more alcohols iv), wherein the one or more compounds v) is chosen from:
a) organic acids;
b) amphoteric or zwitterionic compounds chosen from phospholipids, amino acids, amphoteric or zwitterionic surfactants, or mixtures of two or more thereof;
c) esters, amides, imines, or mixtures of two or more thereof;
d) organic amines;
e) compounds comprising a saturated or unsaturated, linear or branched hydrocarbon-based chain comprising from 1 to 30 carbon atoms; or mixtures of two or more thereof;
wherein composition (B) comprises:
one or more compounds ii) chosen from carbonates, carbonate-generating systems, bicarbonates, bicarbonate-generating systems, or mixtures of two or more thereof; and
one or more silicates iii);
optionally one or more coloring agents; and
optionally the one or more alcohols iv) and/or the one or more compounds v); and
wherein composition (C) comprises:
one or more coloring agents; and
optionally the one or more alcohols iv) and/or the one or more compounds v);
wherein at least one of the compositions (A) or (B) or at least one of the compositions (A) or (B) or (C), if composition (C) is present, comprises the one or more alcohols iv); and
wherein at least one of the compositions (A) or (B) or at least one of the compositions (A) or (B) or (C), if composition (C) is present, comprises the one or more compounds v); and
(2) applying the mixture to the keratin fibers.

50. A multi-compartment device comprising:
a first compartment comprising a composition (A); and
a second compartment comprising a composition (B); and
optionally a third compartment comprising a composition (C);
wherein composition (A) comprises:
one or more chemical oxidizing agents chosen from hydrogen peroxide, hydrogen peroxide-generating systems other than peroxygenated salts, or mixtures thereof; and
optionally one or more alcohols iv) and/or one or more compounds v) different from the one or more alcohols iv), wherein the one or more compounds v) is chosen from:
a) organic acids;
b) amphoteric or zwitterionic compounds chosen from phospholipids, amino acids, amphoteric or zwitterionic surfactants, or mixtures of two or more thereof;
c) esters, amides, imines, or mixtures of two or more thereof;
d) organic amines;
e) compounds comprising a saturated or unsaturated, linear or branched hydrocarbon-based chain comprising from 1 to 30 carbon atoms; or mixtures of two or more thereof; and
wherein composition (B) comprises:
one or more compounds chosen from carbonates, carbonate-generating systems, bicarbonates, bicarbonate-generating systems, or mixtures of two or more thereof; and
one or more silicates;
optionally one or more coloring agents; and
optionally the one or more alcohols iv) and/or the one or more compounds v); and
wherein composition (C) comprises:
one or more coloring agents; and
optionally the one or more alcohols iv) and/or the one or more compounds v);
wherein at least one of the compositions (A) or (B) or at least one of the compositions (A) or (B) or (C), if composition (C) is present, comprises the one or more alcohols iv); and
wherein at least one of the compositions (A) or (B) or at least one of the compositions (A) or (B) or (C), if composition (C) is present, comprises the one or more compounds v).
